(19) United States
(12) Patent Application Publication
(10) Pub. No.: US 2025/0049859 A1
(43) Pub. Date: Feb. 13, 2025

(54) COMPOSITIONS AND METHODS FOR ACTIVATING NK CELLS

(71) Applicant: The Regents of the University of California, Oakland, CA (US)

(72) Inventor: Anahid Jewett, Los Angeles, CA (US)

(21) Appl. No.: 18/810,813

(22) Filed: Aug. 21, 2024

Related U.S. Application Data
(63) Continuation of application No. 18/209,148, filed on Jun. 13, 2023.
Application No. 18/209,148 is a continuation of application No. 16/486,123, filed on Aug. 14, 2019, now abandoned.
(60) Provisional application No. 62/459,397, filed on Feb. 15, 2017.

(51) Int. Cl. A61K 35/32; A61K 35/741; A61K 39/00; C12N 5/078; C12N 5/0783

(52) U.S. Cl. CPC A61K 35/32; A61K 35/741; A61K 39/4613; A61K 39/4644; C12N 5/0643; C12N 5/0646; C12N 2500/72

(57) ABSTRACT

The present application relates to methods of activating a NK cells in vitro, ex vivo, and/or in vivo by an osteoclast cell (OC) and/or a dendritic cell, and methods of treating disease using these activated NK cells.

### CROSS-REFERENCE TO RELATED APPLICATIONS

This application is a continuation of U.S. patent application Ser. No. 18/209,148, filed on Jun. 13, 2023, which is a continuation of U.S. patent application Ser. No. 16/486,123, filed Aug. 14, 2019, which is a national-stage filing under 35 U.S.C. § 371 of PCT Application No. PCT/US2018/18389, filed Feb. 15, 2018, which claims the benefit of priority to U.S. Provisional Patent Application Ser. No. 62/459,397, filed Feb. 15, 2017. The entire contents of each of said applications are incorporated herein by reference in their entirety.

### BACKGROUND OF THE INVENTION

Natural killer (NK) cells lyse and differentiate cancer stem cells/undifferentiated tumors with lower expression of MHC class I, CD54 and B7H1 and higher expression of CD44. Medium and high cytotoxic activity of peripheral-blood lymphocytes are associated with reduced cancer risk, and high NK-cell infiltration of the tumor is associated with a better prognosis, whereas low activity is associated with increased cancer risk.

Suppression of NK cells is mediated by downregulation of NK receptors in the tumor microenvironment. Function of NK cells was shown previously to be significantly reduced in tumor patients. Several in vitro NK expansion techniques have been developed to allow for a higher therapeutic cell dose. The stimulation of peripheral blood mononuclear cells (PBMCs) or purified population of NK cells with feeder cells such as K562 cells expressing interleukin (IL)-15 and 41BB ligand, EBV-TM-LCL, Wilm's tumor or irradiated PBMCs have resulted in greater numbers of NK cells with adequate function. The generated NK cells expressed higher levels of NKG2D, natural cytotoxicity receptors, DNAM-1, and ICAM-1. Thus, various methods to obtain ex vivo-expanded, activated, and CD3+ T cell-depleted NK cells have been established for clinical use. In addition, it has been established that the safety and efficacy of adoptive cellular transfer of HLA-haploidentical NK cells in patients with advanced cancer. Additionally, clinical trials have shown that allogeneic NK cells play a therapeutic role in solid tumors, and are safe for transfer into patients.

Immunotherapy with NK cells has been limited due to inability to obtain sufficient numbers of highly functional NK cells. In addition, unlike NK cells from healthy individuals, expansion of patient NK cells, similar to those from tumor-bearing humanized mice, is significantly limited due to the expansion of a small fraction of contaminating T cells which crowd out NK cells by their faster proliferating capability.

The underlying mechanism of NK cell immunomodulation is not understood. A great need exists to identify therapeutic compositions and methods for improved NK immunotherapy.

### SUMMARY OF THE INVENTION

The present invention is based, at least in part, on the discovery that osteoclasts can induce expansion of NK cells, which further increases CD8+/CD4+ T cell ratio in both healthy humans and cancer patients. While cancer patients generally have more NK cells and CD8+/CD4+ T cell ratio in vivo relative to healthy humans, the excess NK and CD8+ T cells are short-lived (due to the expansion of contaminating T cells which may suppress NK cell function) and lack activity (e.g., the cytoxicity and cytokine selection). However, osteoclasts can induce NK cell expansion and increase both the cell number and the function of NK cells in cancer patients (e.g., measured by their cytokine secretion ability). Dendritic cells preferentially promote the expansion of T cells, whereas osteoclasts preferentially promote the expansion of NK cells, suggesting microenvironmental differences for the selective expansion of T and NK cells. Therefore, the present invention provides a method to expand large numbers of activated NK cells for use in immunotherapeutic strategies. Such cells can be employed to inhibit or eliminate cancer stem cells and control tumor growth by promoting differentiation of stem-like/poorly differentiated tumors

A method of activating a NK cell in vitro or ex vivo, comprising culturing the NK cell in a medium together with an osteoclast cell (OC) is provided herein. The NK may be a primary NK cell, optionally where it has not been transformed. The activated NK cell may expand about 10, 11, 12, 13, 14, 15, 16, 17, 18, 19, 20, 21, 22, 23, 24, 25, or more population doublings within 4 weeks. The culture may comprise a plurality of osteoclast cells (OCs) and a plurality of NK cells, e.g., wherein the ratio of OCs:NK cells in the cell culture is at least 1:2. The osteoclast cells may enhance NK cell cytotoxicity, e.g., as measured by the lysis of oral squamous carcinoma stem-like cells (OSCSCs) by the NK cell or a ⁵¹Cr release cytotoxicity assay.

Additionally, the osteoclast cell may enhance production, secretion, and/or function of at least one cytokine or chemokine produced by the NK cell. For example, the osteoclast cells may enhance secretion of IFN-γ and/or IL-12 by the NK cell, and/or the expression of one or more of NKG2D, NKp46, NKp44, NKp30, CD94, KIR2, and KIR3 by the NK cell.

The NK cell may be a cell purified from a cancer sample of a human subject. In certain embodiments, the cell culture further comprises a T cell also originating from the cancer sample. In certain such embodiments, the NK cell may be preferentially expanded relative to the T cell. The NK cell may be expanded for any length of time, e.g., at least one month. The culture medium may be supplemented with at least one osteoclast cell to continue preferentially expanding the NK cells. The T cell may secrete IFN-γ but may not mediate cytotoxicity, e.g., as measured by the lysis of OSCSCs by the T cell, e.g., in a ⁵¹Cr release cytotoxicity assay. The expanded NK cell may be capable of expanding CD8+ T cells. The NK cell expanded by the OC may also be capable of preferentially expanding CD8+ T cells relative to CD4+ T cells. In certain embodiments, the method further comprises adding an anti-CD3 antibody to the cell culture, e.g., to further enhance secretion of IFN-γ by the NK cell. The activated NK cell may be split anergized.

In certain embodiments, the method may further comprise adding to the cell culture a composition comprising at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain may be either alive or sonicated. For example, the composition may comprise Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus. Alternatively or additionally, the composition may comprise sAJ2 bacteria. The ratio of the sAJ2 bacteria concentration to the NK cell and/or the OC concentrations in the cell culture may be, for example, i) at least 1:2 for NK cells:sAJ2; ii) at least 1:4 for OCs:sAJ2; and/or iii) at least 1:2:4 for OCs:NK cells:sAJ2.

In certain embodiments, the method may further comprise adding to the cell culture another agent capable of activating NK cells.

In certain preferred embodiments, the method comprises: i) providing a cell culture comprising an osteoclast cell (OC), an NK cell, and a T cell; and ii) culturing the NK cell, the T cell, and the osteoclast cell in the cell culture, thereby preferentially activating the NK cell relative to the T cell.

Similarly, provided herein is a method comprising: i) providing a cell culture comprising a dendritic cell (DC), a NK cell and a T cell; and ii) culturing the NK cell, the T cell, and the dendritic cell in the cell culture, thereby preferentially activating the T cell relative to the NK cell. In an embodiment, the NK cell may be a primary NK cell, optionally wherein the primary NK cell has not been transformed. The culture may comprise a plurality of osteoclast cells (OCs) and a plurality of NK cells, e.g., wherein the ratio of OCs:NK cells in the cell culture is at least 1:2. The osteoclast cells may enhance NK cell expansion, and/or the secretion of IL-15 by the NK cell. The activated NK cell may expand to at least about 10, 11, 12, 13, 14, 15, 16, 17, 18, 19, 20, 21, 22, 23, 24, 25, or more population doublings within 4 weeks. Additionally, the osteoclast cell may enhance NK cell cytotoxicity, e.g., as measured by the lysis of oral squamous carcinoma stem-like cells (OSCSCs) by the NK cell. The cell cytotoxicity may be measured by a ⁵¹Cr release cytotoxicity assay.

In certain embodiments, the osteoclast cell (OC) may enhance production, secretion, and/or function of at least one cytokine or chemokine produced by the NK cell. For example, the osteoclast cell may enhance secretion of IFN-γ and/or IL-12 by the NK cell. The osteoclast cell may enhance expression of one or more of NKG2D, NKp46, NKp44, NKp30, CD94, KIR2, and KIR3 by the NK cell. The NK cell and/or the T cell may be purified from a cancer sample from a subject, e.g., a human subject. In certain embodiments, the preferential activation of the NK cell may last for at least one month. Additionally, after the preferential activation of the NK cells attenuates or stops, at least one osteoclast cell may be added to the cell culture for at least one month after culturing the NK cell, thereby continuing activation of the NK cell. In some embodiments, the T cell may secrete IFN-γ but may not mediate cytotoxicity. The cytotoxicity may be measured by the lysis of OSCSCs by the T cell, e.g., by a ⁵¹Cr release cytotoxicity assay. The expanded NK cell may be capable of expanding CD8+ T cells, and may be capable of preferentially expanding CD8+ T cells relative to CD4+ T cells. In certain embodiments, the NK cell expanded by the DC may be capable of preferentially expanding CD4+ T cells against CD8+ T cells. In other embodiments, anti-CD3 antibody may be added to the cell culture, e.g., further enhance secretion of IFN-γ by the NK cell. Additionally, the activated NK cell may be split anergized.

In certain embodiments, the method may further comprise adding to the cell culture a composition comprising at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated. For example, the composition may comprise Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus. Alternatively or additionally, the composition may comprise sAJ2 bacteria. In certain such embodiments, the ratio of the sAJ2 bacteria concentration to the NK cell and/or the OC concentrations in the cell culture may be i) at least 1:2 for NK cells:sAJ2; ii) at least 1:4 for OCs:sAJ2; and/or iii) at least 1:2:4 for OCs:NK cells:sAJ2.

Additionally, the method may further comprise adding to the cell culture another agent that may be capable of activating NK cells. The method may further comprise adding to the cell culture another agent capable of activating T cells.

Similarly, provided herein is a method of treating cancer or a cancer-related disease or disorder in a subject having or suspected of having a cancer or cancer-related disease or disorder by administering to the subject a therapeutically effective amount of osteoclast cells (OCs), a cell culture comprising osteoclast cells (OCs), and/or the supernatant of a cell culture comprising osteoclast cells (OCs).

The osteoclast cells may enhance NK cell expansion in the subject, optionally wherein the osteoclast cells enhance the secretion of IL-15 by the NK cells. In certain embodiments, the the enhanced NK cell expansion may be at least about 10, 11, 12, 13, 14, 15, 16, 17, 18, 19, 20, 21, 22, 23, 24, 25, or more population doublings within 4 weeks. Additionally, the osteoclast cell may enhance NK cell cytotoxicity, e.g., as measured by the lysis of oral squamous carcinoma stem-like cells (OSCSCs) by the NK cell. The cell cytotoxicity may be measured by a ⁵¹Cr release cytotoxicity assay.

In certain embodiments, the osteoclast cells may increase or promote production, secretion, and/or function of at least one cytokine or chemokine produced by the NK cell. For example, the osteoclast cell may enhance secretion of IFN-γ and/or IL-12 by the NK cells. The osteoclast cell may preferentially activate NK cells relative to T cells and/or preferentially enhance expansion of NK cells relative to T cells. In certain embodiments, the preferential expansion of the NK cell may last for at least one month. The T cell may secrete IFN-γ but not mediate cytotoxicity of the cancer, e.g., as measured by the lysis of OSCSCs by the T cell in a ⁵¹Cr release cytotoxicity assay.

In certain embodiments, the activated NK cells may expand CD8+ T cells in the subject, e.g., may preferentially expand CD8+ T cells relative to CD4+ T cells. The subject may also be treated with anti-CD3 antibody to further enhance secretion of IFN-γ by the NK cell. The activated NK cell may be split anergized.

In certain embodiments, the treatment method may further comprise administering to the subject a composition comprising at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated. For example, the composition may comprise Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus. In certain embodiments, the composition may comprise sAJ2 bacteria.

In certain embodiments, the method may further comprise adding to the cell culture another agent capable of activating NK cells.

In certain embodiments, the osteoclast cells, the cell culture, and/or the supernatant may be administered in a pharmaceutical composition, that may be administered systemically or locally to the cancer. In certain embodiments, the osteoclast cells, the cell culture, and/or the supernatant may be administered at least twice to the subject, e.g., the osteoclast cells, the cell culture, and/or the supernatant may be administered to the subject after at least one month since the first administration.

In some embodiments, subject may be human.

### BRIEF DESCRIPTION OF THE DRAWINGS

FIG. 1A-FIG. 1D show the higher expression of NK activating ligands by Osteoclasts. To generate osteoclasts (OCs), monocytes were cultured in medium containing Macrophage colony-stimulating factor (M-CSF) (25 ng/ml) and RANKL (25 ng/ml) for 21 days. Highly purified NK cells (1×10⁶ cells/ml) were treated with the combination of IL-2 (1000 U/ml) and anti-CD16 mAb (3 μg/ml) for 18 hours before they were co-cultured with autologous OCs in the presence or absence of sAJ2 bacteria at 1:2:4 ratios (OCs:NK:sAJ2), respectively. Surface expression of CD3, CD16, and CD56 was analyzed in 1×10⁴ lymphocyte samples from co-cultures at days 6, 9, 12, 15, 19, 24, 29, and 34 using flow cytometry, and culture medium was refreshed and supplemented with rh-IL-2 (1000 U/ml) (FIG. 1A). Cells were co-cultured as described in FIG. 1A and expanded lymphocytes were counted manually using microscope (FIG. 1B). Monocytes were isolated from the PBMCs of a healthy donor. To generate dendritic cells, monocytes were cultured in medium containing GM-CSF (150 ng/ml) and IL-4 (50 ng/ml) for 8 days. Osteoclasts were generated and NK cells were purified as described in FIG. 1A, before they were co-cultured with autologous cells in the presence of sAJ2 bacteria at 1:2:4 ratios (target cells:NK:sAJ2), respectively. On day 6 of the culture, the culture media was removed and the NK cells were treated with rh-IL-2 for 5 hours before the supernatants were harvested, and IFN-γ secretions were determined using single ELISA (FIG. 1C). Monocytes were isolated, dendritic cells and osteoclasts were generated as described in FIG. 1A and FIG. 1D, OSCSCs and K562 tumor cell lines were cultured as described in materials and methods, 1×10⁴ cells were used to analyze MHC-1, CD54, KIR2, KIR3, KLRG1 and MICA/B surface expressions, employing PE-conjugated antibodies and flow cytometry. IgG2 isotype was used as a control (FIG. 1D).

FIG. 2A-FIG. 2J show preferential expansion and significant gain in function of NK cells by osteoclasts and T cells by dendritic cells. Monocytes were purified from human PBMCs and were cultured with GM-CSF (150 ng/ml) and IL-4 (50 ng/ml) for 8 days to generate DCs. To generate osteoclasts, monocytes were cultured in alpha-MEM media containing M-CSF (25 ng/ml) and RANKL (25 ng/ml) for 21 days. For expansion, purified NK cells (1×10⁶ cells/ml) were treated with the combination of IL-2 (1000 U/ml) and anti-CD16 mAb (3 μg/ml) for 18 hours before they were co-cultured with autologous monocytes, DCs or OCs in the presence of sAJ2 at 1:2:4 ratios (monocytes, DCs or OCs:NK:sAJ2). Surface expression of CD3, CD16, and CD56 was analyzed at days indicated in the figure using flow cytometry, and culture medium was refreshed and supplemented with rh-IL-2 (1000 U/ml) (FIG. 2A). Cells were co-cultured as described in FIG. 2A and the numbers of expanded lymphocytes were assessed using microscopic determination (FIG. 2B). The numbers of NK cells (FIG. 2C) and T/NKT (FIG. 2D) cells were determined using the percentages of NK and T/NKT cells (FIG. 2A) within the total expanding cells in FIG. 2B. Cells were co-cultured as described in FIG. 2A and cytotoxicity were determined on the days shown in the figure using a standard 4-hour ⁵¹Cr release assay against the oral squamous cell carcinoma stem cell line (OSCSCs). The lytic units 30/10⁶ cells were determined using the inverse number of lymphocytes required to lyse 30% of OSCSCs×100 (FIG. 2E). Supernatants were harvested from the co-culture of NK with OCs as described in FIG. 1A on days 6, 9, 12 and 15, and IFN-γ secretion was determined using single ELISA (FIG. 2F). NK cells were co-cultured with autologous osteoclasts and expanded from 10 healthy donors as described in FIG. 2A. Cumulative fold expansion of NK cells was calculated for each donor for 31 days (FIG. 2G), and population doubling was calculated based on the log of the ratio of the final count to the baseline count divided by the log of 2 (FIG. 211). Dendritic cells and osteoclasts were generated as described in FIG. 2A and 1×10⁴ cells were used to analyze ULBPs, KIR2, KIR3, KLRG1 and MICA/B surface expressions using PE-conjugated antibodies and flow cytometric analysis. IgG2 isotype control antibody was used as control (FIG. 2I). Freshly isolated NK cells (upper row) and NK cells co-cultured with autologous osteoclasts and expanded as described in FIG. 2A (lower row) were used to analyze CD16, Nkp30, Nkp44, Nkp46, KIR2, KIR3, CD94, and NKG2D surface expression using, PE-conjugated antibodies. IgG2 isotype control antibody was used as control (FIG. 2J).

FIG. 3A-FIG. 3H show that unlike NK cells, T cells purified from osteoclast-expanded NK cells do not mediate cytotoxicity against OSCSCs and secrete IFN-γ moderately. Freshly purified NK cells were treated and co-cultured with monocyte-derived autologous osteoclasts as described in Materials and Methods. Surface expression of CD3, CD16, CD56, GL3 (TCR γ/δ), CD4 and CD8 was analyzed in lymphocyte samples from co-cultures at day 9 using FITC- and PE-conjugated antibodies and flow cytometry (FIG. 3A). NK cells were treated and co-cultured with autologous osteoclasts as described in FIG. 1A and on day 9, CD3T-positive cells were sorted out using CD3T positive selection kit, purity of CD3T-negative (NK) cells was assessed using CD3, CD16, CD56 FITC and PE-conjugated antibodies and flow cytometry (FIG. 3B). CD3T-positive cells and CD3T-negative cells (CD16 positive cells) were treated with rh-IL-2 (1000 U/ml) for 18-20 hours before they were tested for cytotoxicity using a standard 4-hour 51Cr release assay against the OSCSCs (FIG. 3C) and K562 (FIG. 3D) cell lines. The lytic units 30/10⁶ cells were determined using the method described in Materials and Methods, for OSCSCs and K562 respectively. The supernatant was harvested from the culture and IFN-γ secretion was determined using single ELISA (FIG. 3E). NK cells, CD3T, CD4T, CD8T, and T6T cells were purified from PBMC as described in materials and methods, and were activated with rh-IL-2 for 18-20 hours, before they were tested for cytotoxicity using a standard 4-hour 51Cr release assay against the OSCSCs (FIG. 3F). The lytic units 30/10⁶ cells were determined using the inverse number of lymphocytes required to lyse 30% of OSCSCs×100 (FIG. 3F). NK and T cells were purified from PBMCs as described in materials and methods, NK cells were treated as described in Materials and Methods. T cells were activated with anti-CD3 (1 μg/ml) and anti-CD28 (3 μg/ml) 18-20 hours before they were cultured with autologous OCs, and expanded lymphocytes were counted manually using microscope day 4 after the culture (FIG. 3G). NK and T cells were purified and cultured with OCs, and counted on day 4 as described in FIG. 3G, fold expansion of lymphocytes expanded by the OCs were divided by fold expansion of lymphocytes without the OCs (FIG. 311).

FIG. 4A-FIG. 4S show reduced proportions of NK cells, NK cell-mediated cytotoxicity, and IFN-γ secretion with each successive re-stimulation of NK cell cultures with osteoclasts and sAJ2 bacteria. Freshly purified NK cells were treated and co-cultured with monocyte-derived autologous osteoclasts as described in FIG. 2A. Surface expression of CD3, CD16 and CD56 was analyzed in 1×10⁴ lymphocyte from co-cultures at days indicated in the figure using flow cytometric analysis (FIG. 4A). After 36 days, when NK cells ceased to expand, they were re-cultured with fresh autologous osteoclasts as described in FIG. 2A. Surface expression of CD3, CD16, and CD56 was analyzed on the days indicated in the Figure using antibody staining and flow cytometric analysis (FIG. 4B). On day 63, when cells ceased to expand, they were re-cultured with OCs as described and surface expression of CD3, CD16 and CD56 was analyzed on days shown in the Figure (FIG. 4C). The numbers of expanded lymphocytes were assessed using microscopic determination (FIG. 4D, 4G, 4J) and the numbers of NK cells (FIGS. 4F, 4I, 4L) and T/NKT (FIG. 4E, 4H, 4K) cells were determined using the percentages of NK and T/NKT cells within the total expanding cells (FIG. 4D). Cell death was determined in lymphocytes at days 36, 59 and 83 using propodium iodide staining and flow cytometric analysis (FIG. 4M). Freshly purified NK cells were treated and co-cultured with autologous osteoclasts as described in FIG. 2A. Lymphocytes were then tested for cytotoxicity using a standard 4-hour ⁵¹Cr release assay against the OSCSCs after 6, 17, and 34 days of the co-culture (FIG. 4N), 40 and 63 days of the co-culture (FIG. 4O) or 76 and 92 days of the co-culture (FIG. 4P). The lytic units 30/10⁶ cells were determined using the method described in FIG. 2E. The supernatants were harvested and IFN-γ secretion was determined using single ELISA using supernatants from days 6, 9, 12, 15, 18 and 21, (FIG. 4Q); days 40, 46, 51, 55, and 59 (FIG. 4R), and days 76, 83, 92, and 97 (FIG. 4S).

FIG. 5A-FIG. 5H show that osteoclasts, but not K562 or OSCSCs, expand NK cells and increase NK cell function substantially. To generate osteoclasts, monocytes were cultured in medium containing M-CSF (25 ng/ml) and RANKL (25 ng/ml) for 21 days, K562 tumor cell lines were cultured as described in materials and methods. Highly purified NK cells (1×10⁶ cells/ml) were treated with the combination of IL-2 (1000 U/ml) and anti-CD16mAb (3 μg/ml) for 18 hours before they were co-cultured with K562 and autologous OCs in the presence of sAJ2 bacteria at 1:2:4 ratios (OCs:NK:sAJ2), respectively. Cells from the cultured were counted manually using microscope on day 6, 10 and 13 (FIG. 5A). The osteoclasts generated as described in FIG. 1A and K562 tumor cells lines were irradiated at 40 grays (Gy) as described in the materials and methods. NK cells were purified and treated as described in Materials and Methods, before they were co-cultured with irradiated K562 and irradiated autologous OCs in the presence of sAJ2 bacteria at 1:2:4 ratios (OCs:NK:sAJ2), respectively. Cells from the cultured were counted manually using microscope on day 6, 10 and 13 (FIG. 5B). NK cells were purified and cultured with OCs and K562 as described in FIG. 1A, cytotoxicity of lymphocytes co-cultured for 6 days was determined using a standard 4-hour 51Cr release assay against OSCSCs. The lytic units 30/10⁶ cells were determined using method described in FIG. 3F (FIG. 5C). NK cells were purified and cultured with OCs and K562 as described in FIG. 5B, cytotoxicity of lymphocytes co-cultured for 6 days was determined using a standard 4-hour 51Cr release assay against OSCSCs. The lytic units 30/10⁶ cells were determined using method described in FIG. 3F (FIG. 5D). NK cells were purified and cultured with OCs and OSCSCs as described in FIG. 5A, cytotoxicity of lymphocytes co-cultured for 6 days was determined using a standard 4-hour ⁵¹Cr release assay against OSCSCs. The lytic units 30/10⁶ cells were determined using method described in FIG. 3F (FIG. 5E). NK cells were purified and cultured with OCs and K562 as described in FIG. 3A, supernatant was harvested on day 3, 6, 7, 10 and 13, and IFN-γ secretion was determined using single ELISA (FIG. 5F). NK cells were purified and cultured with irradiated OCs and irradiated K562, supernatant was harvested on day 3, 6, 7, 10 and 13, and IFN-γ secretion was determined using single ELISA (FIG. 5G). NK cells were purified and cultured with OCs and OSCSCs, supernatant was harvested on day 1, 3, 6 and 8, and IFN-γ secretion was determined using single ELISA (FIG. 511).

FIG. 6A-FIG. 6P show that purified NK cells cultured with OCs from cancer patients expand more T cells than NK cells, mediate much lower cytotoxicity, and cytokine secretion compared to those expanded from healthy donors. Freshly purified NK cells from healthy donor and cancer patient were treated and co-cultured with monocyte-derived OCs as described in FIG. 2A. Surface expression of CD3, CD16 and CD56 was analyzed on expanding cells at days 6, 9, 12, 15, 18, 21, 24, 27, and 31 of cancer patient (FIG. 6A) and healthy donor (FIG. 6B) using antibody staining followed by flow cytometric analysis. Cell death was determined on expanding NK cells from cancer patients and healthy donors at day 19 using PI staining and flow cytometric analysis (FIG. 6C). After 6, 9, 12, 15, 18, 21, 24, 27, and 31 days of co-culture, expanded lymphocytes were manually counted using microscopy (FIG. 6D). The numbers of NK cells (FIG. 6E) and T/NKT (FIG. 6F) cells were determined using the percentages of NK and T/NKT cells within the total expanding cells in FIG. 6A and FIG. 6B. Cytotoxicity of lymphocytes was determined on days 12 and 15 using standard 4-hour ⁵¹Cr release assay against OSCSCs. The lytic units 30/10⁶ cells were determined using the method described in FIG. 2E (FIG. 6G). Lytic units from FIG. 6G were normalized based on per NK cells (FIG. 611). The supernatants were harvested from the overnight, day 6, 9, 12, 15, 18, 21, 24, and 27 co-cultures and IFN-γ (FIG. 6I), IL-10 (FIG. 6J), and IL-6 (FIG. 6K) secretion was determined using single ELISAs. Freshly purified NK cells from healthy donors, cancer patient with tonsillar carcinoma (patient #1) and pancreatic cancer (patient #2) were treated and co-cultured with OCs as described in FIG. 2A. Surface expressions of CD3, CD16, and CD56 were analyzed on lymphocytes from co-cultures of day 21 for healthy donor and patient NKs, and day 87 (3rd stimulation) of healthy donors (FIG. 6L), and surface expression of Nkp30, Nkp44, Nkp46, KIR2, KIR3, CD94 and NKG2D was analyzed within CD16 positive cells (FIG. 6M). IgG2 isotype control antibodies were used as control (FIG. 6L and FIG. 6M). The supernatants were harvested from the co-cultures on day 13, and the equal amounts of supernatants (200 μl) from each donor was used to differentiate OSCSCs for overnight, before the levels of MHC-I, CD54, CD44 and B7H1 surface expressions were determined on OSCSCs. IgG2 isotype control antibodies were used as controls (FIG. 6N). Cell death was determined in untreated and NK cell supernatant-differentiated OSCSCs using propodium iodide staining and flow cytometric analysis (FIG. 6O). Highly purified NK cells were treated with IL-2 (1000 U/ml) and used to determine cytotoxicity against untreated and NK supernatant-differentiated OSCSCs in 4-hour ⁵¹Cr release assay. The lytic units 30/10⁶ cells were determined using the method described in FIG. 2E (FIG. 6P).

FIG. 7A-FIG. 7K show that small fraction of contaminating T cells within purified NK cells from cancer patient expand faster and crowd out NK cells likely due to decreased NK cell function. Freshly purified NK cells from a healthy donor and a pancreatic cancer patient were treated and co-cultured with monocyte-derived allogeneic (from different healthy donor) osteoclasts as described in FIG. 1A. Surface expression of CD3, CD16 and CD56 was analyzed in 1×10⁴ lymphocyte samples from co-cultures at days 6, 10, 13, 17, 21, 24, 28, 32 and 36 of cancer patient (FIG. 7A) and healthy donor (FIG. 7B) using FITC- and PE-conjugated antibodies and flow cytometry. After 6, 10, 13, 17, 21, 24, 28 and 32 days of co-culture, expanded lymphocytes were counted manually using microscope (FIG. 7C). Cells counted as mentioned in FIG. 7C and were adjusted based on the surface expression analyzed in FIGS. 7A and 7B to determine the number T/NKT cells (FIG. 7D) and NK cells at each day (FIG. 7E). Cytotoxicity of lymphocytes co-cultured for 18-20 hours, 13, 20 and 32 days was determined using a standard 4-hour ⁵¹Cr release assay against OSCSCs. The lytic units 30/10⁶ cells were determined using method described in FIG. 3F (FIG. 7G). Lytic units from FIG. 7F was adjusted based on surface expression analyzed is FIG. 7A and FIG. 7B to determine the cytotoxicity mediated by 1 NK cell against OSCSCs (FIG. 711). The supernatant was harvested from the overnight, 6, 10, 13, 17, 21, 24, 28 and 32 days of co-culture and IFN-γ (FIG. 7I), IL-10 (FIG. 7J), and IL-6 (FIG. 7K) secretion was determined using single ELISA.

FIG. 8 shows phenotype of CD3 T cell depleted lymphocytes from the splenocytes of hu-BLT mice. Humanized-BLT (hu-BLT; human bone marrow/liver/thymus) mice were generated by surgical implantation of human fetal liver and thymus tissue under the renal capsule of 6-8 weeks old immunocompromised NOD.CB17-Prkdcscid/J and NOD.Cg-Prkdcscid Il2rgtm1Wjl/SzJ (NSG) mice. 4-6 weeks post tissue transplant, mice were sub-lethally irradiated and intravenously injected with CD34+ cells isolated from fetal liver to support full reconstitution of human bone marrow. 8-12 weeks after injection with CD34+ cells, reconstitution of human immune system was analyzed using blood specimens. At the end of this experiment engraftment of human immune cells was confirmed by staining splenocytes and bone marrow cells with anti-human CD45, CD3, CD4 and CD8 antibodies and analyzed by flow cytometry (data not shown). Successfully reconstituted hu-BLT mice (levels and lineages of T cells comparable to healthy donors) were orthotopically injected with 1×10⁶ of human OSCSCs into the floor of the mouth. Disease progression and weight loss was monitored for another 3-4 weeks. Animals were sacrificed, spleens were harvested from the sacrificed animals, and single cell suspensions were obtained as described in materials and methods. CD3T cells were sorted out using human CD3T positive selection kit. Flow through cells (CD3-negative cells) were analyzed for surface expression of human CD3, CD16, CD56, CD45, CD19, CD14, after staining with the respective PE-conjugated, PE-Cy5-conjugated and FITC-conjugated antibodies. Isotype control antibodies were used as a control.

FIG. 9A-FIG. 9K show in vitro expanded lymphocytes depleted of T cells from tumor bearing humanized BLT mice expand T cells and contain less NK cells and mediate lower cytotoxicity when compared to those obtained from healthy hu-BLT mice. Reconstituted BLT (levels and lineages of T cells comparable to healthy donors) were orthotopically injected with 1×10⁶ of human OSCSCs into the floor of the mouth. Disease progression and weight loss was monitored for another 4-5 weeks. Mice were sacrificed, the spleens were harvested, and single cell suspensions were obtained as described in supplementary materials and methods. CD3+ T cells were sorted out using positive selection kit and the flow through cells were analyzed for surface expression of human CD3/CD16/CD56 after staining with the respective antibodies. Isotype control antibodies were used as a control (FIG. 9A). CD3-negative cells (1×10⁶ cells/ml) from hu-BLT mice were treated with the combination of IL-2 (1000 U/ml) and anti-CD16mAb (3 μg/ml) for 18 hours before they were cultured with OCs in the presence of sAJ2 at 1:2:4 ratios (OC:NK:sAJ2). Surface expression of CD3, CD16, and CD56 was analyzed on days 6, 10, 14, 18, and 22 using flow cytometric analysis (FIG. 9B). After 6, 10, 18, and 22 days of co-culture, expanded lymphocytes were manually counted using microscopy (FIG. 9C). The numbers of NK cells (FIG. 9D) and T/NKT (FIG. 9E) cells were determined using the percentages of NK and T/NKT cells within the total expanding cells. Cytotoxicity of NK cells co-cultured for 10 and 18 days was determined using standard 4-hour ⁵¹Cr release assay against OSCSCs and the lytic units 30/10⁶ cells were determined using inverse number of NK cells required to lyse 30% of OSCSCs×100 (FIG. 9F). Lytic units was normalized and adjusted per NK cell lysis against OSCSCs (FIG. 9G). The supernatants were harvested from the co-culture on day 6, 10, and 13, and IFN-γ (FIG. 911), IL-10 (FIG. 9I), and IL-6 (FIG. 9J) secretion was determined using single ELISAs. Peripheral blood was collected post-mortem by cardiac puncture from hu-BLT mice and serum samples were harvested and analyzed for IFN-γ, IL-10, and IL-6 secretion using multiplex arrays (FIG. 9K).

FIG. 10A-FIG. 10C show cytokines, chemokines and growth factors and ligands secreted by primary and osteoclast-expanded NK cells. Highly purified NK cells and monocytes were obtained from peripheral blood mononuclear cells (PBMCs) of healthy donors and NK cells were treated (1×10⁶ cells/ml) with IL-2 (1000 U/ml) for 18 hours before the supernatant was harvested. To generate osteoclasts, monocytes were cultured in alpha-MEM media containing M-CSF (25 ng/ml) and RANKL (25 ng/ml) for 21 days. For expansion, purified NK cells (1×10⁶ cells/ml) were treated with the combination of IL-2 (1000 U/ml) and anti-CD16mAb (3 μg/ml) for 18 hours before they were co-cultured with autologous osteoclasts in the presence of sAJ2 bacteria at 1:2:4 ratios (OC:NK:sAJ2), respectively. The supernatant was harvested after 6 days of co-culture and multiplex assay was used to determine cytokines (FIG. 10A), chemokines (FIG. 10B) and growth factors (FIG. 10C) levels secreted by the primary and expanded NK cells.

FIG. 11A-FIG. 11C show blocking IL-12, IL-15, or a combination of both resulted in reduced NK cell expansion, NK cell mediated cytotoxicity and cytokine secretion. Freshly purified NK cells from a healthy donor were treated and co-cultured with autologous osteoclasts as described in FIG. 2A in the presence and absence of anti-IL12, -IL-15, or a combination of anti-IL-12 and -IL-15 mAbs at 100 ng/ml and 1 g/ml respectively. Co-cultures were replenished with IL-2 (1000 units/mL) every 2 days. NK cells were counted using microscopy on days 6, 8, 12, 14, and 20 (FIG. 11A). On day 9 and day 15, 1×10⁵ NK cells from each expanded samples were used in standard 4-hour ⁵¹Cr release against OSCSCs. The lytic units 30/10⁶ cells were determined using inverse number of NK cells required to lyse 30% of OSCSCs×100 (FIG. 11B). The supernatants were harvested from the co-cultures on day 8, 12, 15 and 20, and IFN-γ secretion was determined using single ELISA (FIG. 11C).

FIG. 12A-FIG. 12G show addition of anti-CD3 antibody inhibits T cell expansion and increases OC-expanded NK cells. Freshly purified NK cells from healthy donors and cancer patients expanded with OCs for 27 days before the cultures were treated with rh-IL-2 and anti-CD3 (1 μg/ml), before the numbers of NK cells (FIG. 12A) and T cells (FIG. 12B) were determined by microscopic evaluation on days 29, 31, and 35 (both healthy and patient). Loss of forward and side scatter was determined in healthy cells (FIG. 12C) and patient cells (FIG. 12D) treated as described in FIG. 12A and FIG. 12B. Cells were stained with PI and analyzed for DNA fragmentation (FIG. 12E). Lymphocytes from day 31 culture were used in standard 4-hour ⁵¹Cr release against OSCSCs. The lytic units 30/10⁶ cells were determined using inverse number of NK cells required to lyse 30% of OSCSCs×100 (FIG. 12F). The supernatants were harvested from the co-culture on day 35 and IFN-γ secretion was determined using single ELISA (FIG. 12G).

FIG. 13 shows that purified T cells treated with anti-CD3 mAb in the absence of NK cells did not lose forward and side scatter. Highly purified T cells and monocytes were obtained from peripheral blood mononuclear cells (PBMCs) of healthy donors and T cells were treated (1×10⁶ cells/ml) with IL-2 (100 U/ml) and anti-CD3 (1 μg/ml) for 18 hours before they were co-cultured with autologous osteoclasts in the presence of sAJ2 bacteria at 1:2:4 ratios (OC:T cells:sAJ2), respectively. The cells were analyzed for CD3, CD16 and CD56 on day 9 after the culture. Anti-CD3 treated T cells did not lose Forward and side scatter in the absence of NK cells.

FIG. 14A-FIG. 14H show that osteoclast activated NK cells substantially increase CD8+ T cell numbers. PBMCs from healthy donors and cancer patients were analyzed for the surface expression of CD3, CD4 and CD8 using PE- and FITC-conjugated antibody staining followed by flow cytometric analysis (FIG. 14A). Freshly purified NK cells from healthy donors and cancer patients were treated and co-cultured with OCs as described in FIG. 2A. T cells were purified from PBMC of healthy donors and cancer patients using CD3 positive selection kits, and T cells were activated with combination of rh-IL2 (100 u/ml) and anti-CD3 (1 μg/ml) and anti-CD28 mAbs (1 μg/ml) for 18-20 hours before they were co-cultured with OCs in the presence of sAJ2 at 1:2:4 ratios (OCs:Tcells:sAJ2). Surface expressions of CD3, CD4, and CD8 were analyzed on lymphocytes (FIG. 14B). Monocytes were purified from human PBMCs and OCs and DCs were generated, and purified NK cells were co-cultured as described in FIG. 2A, and the numbers of expanded lymphocytes were assessed using microscopic determination (FIG. 14C), the numbers of T cells (FIG. 14D) and NK cells (FIG. 14E) were determined using the percentages of NK and T cells as described in FIG. 2A, within the total expanding cells in FIG. 14C. Surface expressions of CD3+CD4+, and CD3+CD8+ cells were analyzed on lymphocytes, and the numbers of CD3+CD4+ T cells (FIG. 14F) and CD3+CD8+ T cells (FIG. 14G) were determined using the percentages of CD4 and CD8 cells within the total T cells in panel D. OC activated NK expanded T cells, DC activated NK expanded T cells, OC expanded/activated T cells and DC expanded/activated T cells were stained with the antibodies against CD45RO, CD62L, CD28, CD44, CCR7 and CD127 and analyzed by flow cytometry. The numbers in quadrant 2 represent the percentages of cells positive for each antibody within CD3+ T cells (FIG. 14H).

FIG. 15A-FIG. 15B show that osteoclast-expanded NK cells retain their cytokine secretion and cytotoxic function after freezing. Freshly purified NK cells were treated and co-cultured with monocyte-derived autologous osteoclasts as described in FIG. 1A, day 9 after the cultures, expanded NK cells were frozen. NK cells were thawed and treated with rh-IL-2 (1000 U/ml), day 6 and day 9 after the culture, the supernatant was harvested and IFN-γ secretion was determined using single ELISAs (FIG. 15A). NK cells were cultured as described in FIG. 1A, and the cytotoxicity of lymphocytes day 6 and 9 after the culture was determined using a standard 4-hour ⁵¹Cr release assay against OSCSCs. The lytic units 30/10⁶ cells were determined using method described in FIG. 3F (FIG. 15B)

FIG. 16A-FIG. 16E show the decreased numbers of PBMCs obtained from peripheral blood of pancreatic (FIG. 16B), colon (FIG. 16C), oral (FIG. 16D), and prostate (FIG. 16E) cancer patients. FIG. 16A shows the decrease in healthy subjects versus patients.

FIG. 17A-FIG. 17E show increased percentages of NK and CD14 monocytes but significant decreases of T cells and B cells from PBMCs obtained from peripheral blood of healthy subjects (FIG. 17A), pancreatic (FIG. 17B), colon (FIG. 17C), oral (FIG. 17D), and prostate (FIG. 17E) cancer patients.

FIG. 18A-FIG. 18D show decreased NK cell cytotoxicity by patient NK cells as compared to healthy NK cells. Decreased NK cell-mediated cytotoxicity by all cancer patient NK cells as compared to healthy NK cells (FIG. 18A). Decreased NK cell-mediated cytotoxicity by colon cancer patient NK cells as compared to healthy NK cells (FIG. 18B). Decreased NK cell-mediated cytotoxicity by oral cancer patient NK cells as compared to healthy NK cells (FIG. 18C). Decreased NK cell-mediated cytotoxicity by prostate cancer patient NK cells as compared to healthy NK cells (FIG. 18D).

FIG. 19A-FIG. 19H show osteoclast-expanded NK cells from patients and the cytotoxicity and secretion of IFN-γ. Lower cell counts of osteoclast-expanded NK cells from patients as compared to healthy NK cells (FIG. 19A). Decreased NK cell-mediated cytotoxicity by patients NK cells as compared to healthy NK cells (FIG. 19B). Decreased NK cell-mediated cytotoxicity by osteoclast-expanded NK cells from patients as compared to healthy NK cells (FIG. 19C). Decreased IFN-γ secretion by osteoclast-expanded NK cells from patients as compared to healthy NK cells (FIG. 19D). Lower cell counts, decreased NK cell-mediated cytotoxicity, and decreased IFN-γ secretion by osteoclast-expanded NK cells from pancreatic cancer patients as compared to healthy NK cells (FIG. 19E). Lower cell counts, decreased NK cell-mediated cytotoxicity, and decreased IFN-γ secretion by osteoclast-expanded NK cells from colon cancer patients as compared to healthy NK cells (FIG. 19F). Lower cell counts, decreased NK cell-mediated cytotoxicity, and decreased IFN-γ secretion by osteoclast-expanded NK cells from oral cancer patients as compared to healthy NK cells (FIG. 19G). Lower cell counts, decreased NK cell-mediated cytotoxicity, and decreased IFN-γ secretion by osteoclast-expanded NK cells from prostate cancer patients as compared to healthy NK cells (FIG. 19H).

FIG. 20 shows cytokine secretion in non-osteoclast expanded NK cells from pancreatic cancer patients.

FIG. 21A-FIG. 21E show IFN-γ secretion of osteoclast-expanded T cells from healthy subjects (FIG. 21A), pancreatic (FIG. 21B), colon (FIG. 21C), oral (FIG. 21D), and prostate (FIG. 21E) cancer patients.

FIG. 22A-FIG. 22F show IFN-γ secretion from NK cells, T cells, and osteoclast-expanded NK cells and T cells from NK cells versus T cells (FIG. 22A), healthy subjects (FIG. 22B), pancreatic (FIG. 22C), colon (FIG. 22D), prostate (FIG. 22E), and oral (FIG. 22F) cancer patients.

FIG. 23A-FIG. 23D show T cell secretion of IFN-γ from healthy subjects (FIG. 23A), pancreatic (FIG. 23B), colon (FIG. 23C), and prostate (FIG. 23D) cancer patients.

FIG. 24A-FIG. 24F show the total numbers of expanding T cells versus NK cells activated through effect of T cells activated through surface receptor crosslinking determined within days 0 to 15 in cumulative NK cells versus T cells (FIG. 24A), NK cells from healthy subjects (FIG. 24B), pancreatic (FIG. 24C), colon (FIG. 24D), oral (FIG. 24E), and prostate (FIG. 24F) cancer patients.

FIG. 25A-FIG. 25D show all cancer (FIG. 25A), pancreatic (FIG. 25B), colon (FIG. 25C), and prostate (FIG. 25D) cancer patient capacity to expand T cells compared to healthy individuals' capacity determined within days 0 to day 15.

FIG. 26 shows the decrease in cytokines and chemokines in the serum of patients versus healthy individuals.

FIG. 27A-FIG. 27B show the percentage of CD4 T cells and CD8 T cells in PBMCs from pancreatic (FIG. 27A) and colon (FIG. 27B) cancer patients compared to healthy individuals.

FIG. 28A-FIG. 28E show the ratio of CD4/CD8 T cells within PBMCs of pancreatic (FIG. 28B), colon (FIG. 28C), oral (FIG. 28D), and prostate (FIG. 28E) cancer patients versus healthy individuals (FIG. 28A).

FIG. 29A-FIG. 29E show the ratio of CD4 T cells to CD8 T cells in healthy subjects (FIG. 29A) versus pancreatic (FIG. 29B), colon (FIG. 29C), oral (FIG. 29D), and prostate (FIG. 29E) cancer patients.

FIG. 30A-FIG. 30B show the effect of culturing CD8 T cells and CD4 T cells without (FIG. 30A) and with (FIG. 30B) osteoclasts.

FIG. 31 shows the IFN-γ secretion of NK and CD8 T cells.

FIG. 32 shows CD8 T cell expansion and CD4 T cell expansion promoted by osteoclasts and NK cells respectively.

FIG. 33 shows the cytotoxicity of osteoclast-expanded NK cells against cancer stem cells/undifferentiated tumors.

FIG. 34 shows effect of osteoclast-expanded NK cells on NK cell expansion and the cytotoxic effect of NK cells by osteoclast-expanded cells.

FIG. 35 shows the T cells with effector memory phenotype in OC-expanded NK cells compared to DC-expanded NK cells.

FIG. 36A-FIG. 36C show the number of T cells with exhausted phenotype in OC-expanded versus DC-expanded NK cells. Increased percentages of CD4+, decreased percentages of CD8+ T cells in DC-expanded NK cells versus OC-expanded NK cells (FIG. 36A). Increased number of T cells with exhausted phenotype in OC-expanded versus DC-expanded NK cells (FIG. 36B). Increased number of effector memory T cells in DC-expanded versus OC-expanded NK and T cells (FIG. 36C).

FIG. 37A-FIG. 37B show IFN-γ expressing cells in OC-expanded NK and T cells from pancreatic cancer patients. Decreased IFN-γ spots numbers and size in osteoclast-expanded NK cells from patients as compared to healthy NK cells (FIG. 37A). Decreased IFN-γ spots numbers and size in osteoclast-expanded T cells from patients as compared to healthy T cells (FIG. 37B).

FIG. 38 shows a table of CD8 and NK specific cytokines, co-stimulatory ligand, granzymes, perforin and soluable Fas and Fas ligand secreted by OC-expanded T cells.

FIG. 39 shows a table of CD8 related cytokines, chemokines, co-stimulatory ligands, sFas and Fas Ligand and granzymes and perforin secreted by CD8+ T cells from OC-expanded NK cell culture.

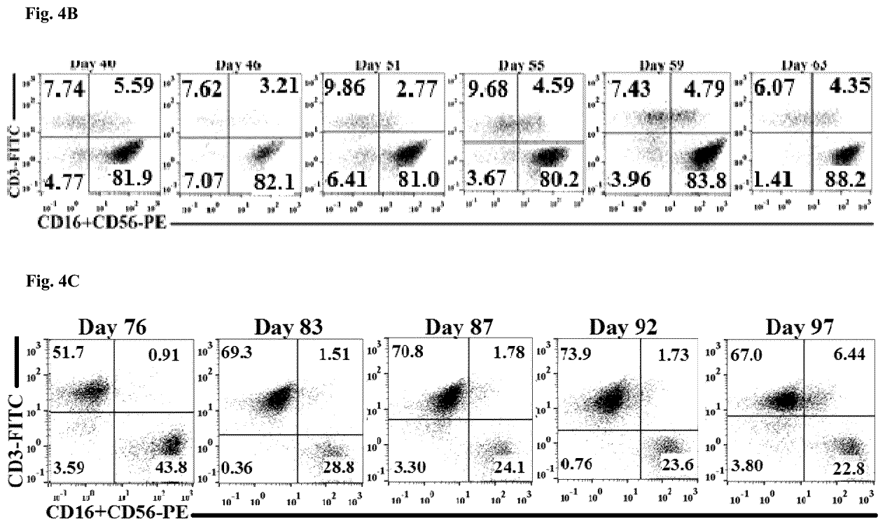

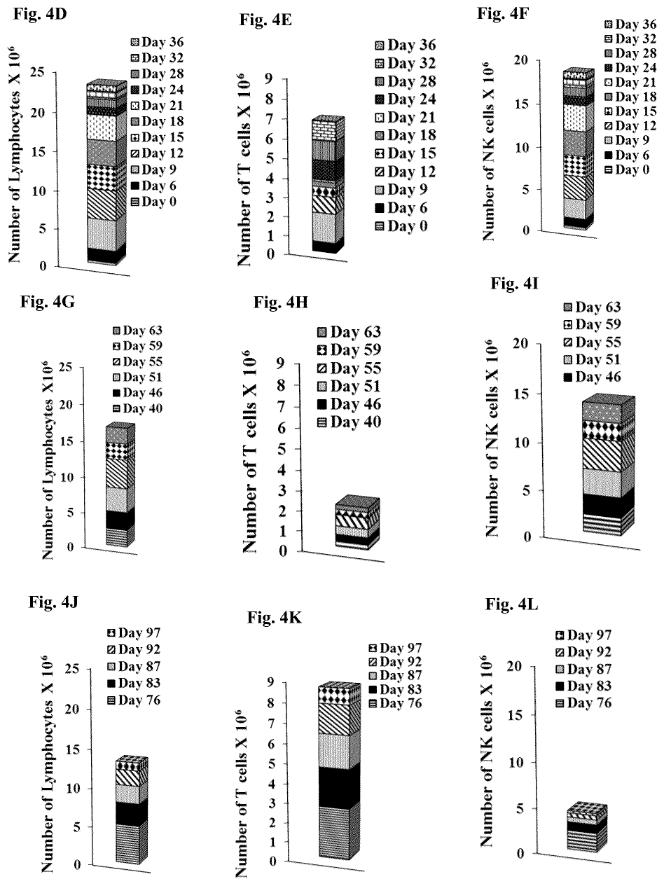

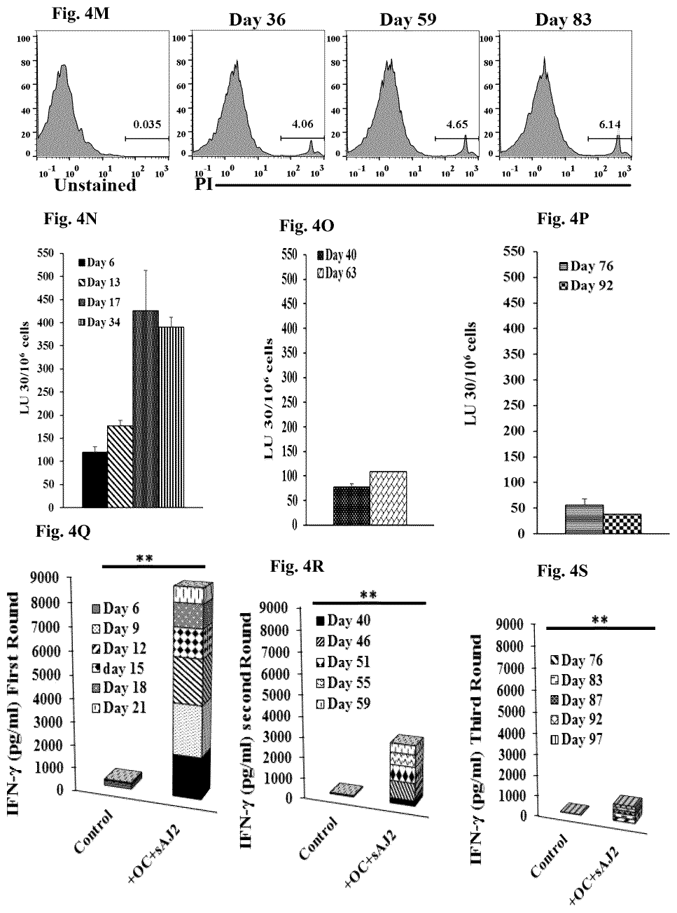

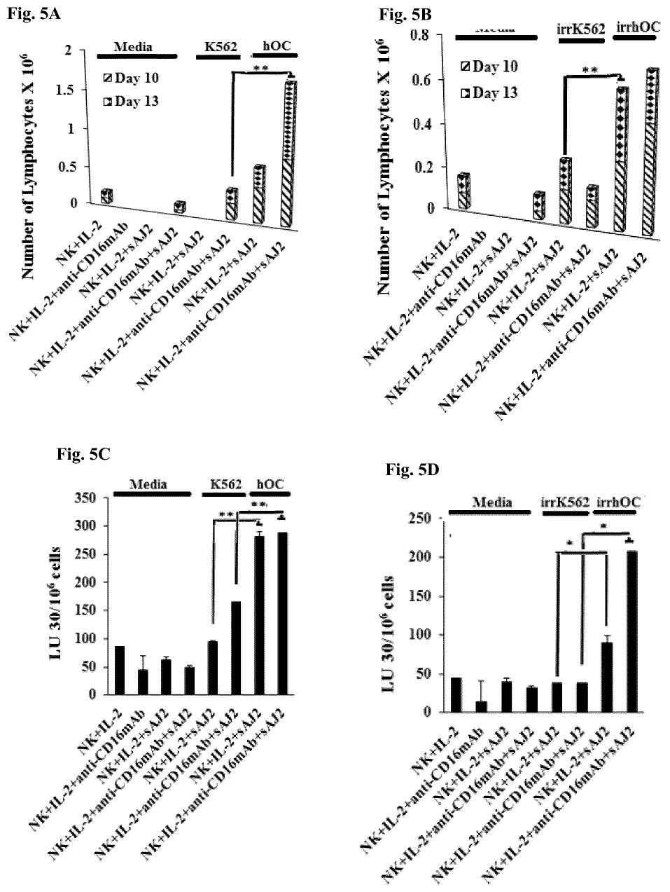

FIG. 4O shows levels of GM-CSF, IFN-g, IL-10, TNF-α, lower co-stimulatory ligand sCD137, granzymes, perforin, soluble Fas, and Fas ligand secreted from NK- or OC-expanded CD8 cells.

FIG. 41 shows cytotoxic activity of NK cells from oral tumor implanted BLT mice as compared to mice with no tumors.

FIG. 42 shows CD8+ T cells in BM, spleen, and blood after immunotherapy with super-charged NK cells after tumor implantation.

FIG. 43 shows the serum level of IL-6, ITAC, GM-CSF, and IL-8 after immunotherapy with super-charged NK cells in BLT mice after tumor implantation.

FIG. 44 shows cytotoxicity of osteoclast-expanded patients' NK cells at different days of expansion.

FIG. 45 shows the number of NK cells in an osteoclast-expanded NK cell population in a patient at different days of expansion.

FIG. 46 shows the number of osteoclast-expanded NK cells compared to DC-expanded cells from day 15 to day 25.

FIG. 47 shows the number of osteoclast-expanded NK cells compared to DC-expanded NK cells at different days of expansion.

FIG. 48 shows the numbers of DC-expanded T cells compared to osteoclast-expanded T cells at different days of expansion.

FIG. 49 shows the cytotoxicity of osteoclast-expanded NK cells compared to DC-expanded NK cells at different days of expansion.

FIG. 50 shows secretion of IFN-γ by primary, non-osteoclast expanded and osteoclast-expanded patient's NK cells compare to healthy donor NK cells at different days of expansion.

FIG. 51 shows IFN-γ secretion by primary, non-osteoclast expanded and osteoclast-expanded patient's T cells compared to healthy donor T cells at different days of expansion.

FIG. 52 shows IFN-γ secretion by osteoclast-expanded patient's NK cells when compared to those obtained from healthy donors' NK cells in different days of expansion.

FIG. 53 shows IFN-γ secretion by primary, non-osteoclast expanded and osteoclast-expanded patient's T cells when compared to those obtained from healthy donors' T cells in some patients at different days of expansion.

FIG. 54 shows IFN-γ secretion (combined secretion from all days of expansion) by primary, non-osteoclast expanded and osteoclast-expanded patient T cells (T cells were positively selected) when compared to those obtained from healthy donors' T cells in different days of expansion.

FIG. 55 shows IFN-γ secretion (combined secretion from all days of expansion) by positively selected primary, non-osteoclast expanded and osteoclast-expanded patient T cells when compared to negatively selected T cells from healthy donors.

FIG. 56 shows IFN-γ secretion per cell basis by osteoclast-expanded T cells (T cells were positively selected) when compared to NK cells obtained from healthy donors. Primary positively selected T cells activated with IL-2 secrete higher IFN-γ when compared to NK cells.

FIG. 57 shows IFN-g secretion per cell basis by primary, non-osteoclast and osteoclast-expanded patient T cells (T cells were positively selected) when compared to T cells obtained from healthy donors.

FIG. 58 shows IFN-g secretion per cell basis by osteoclast-expanded patient T cells (T cells were positively selected) and NK cells. Primary, non-osteoclast expanded T cells with IL-2 have higher IFN-g secretion when compared to primary NK cells treated with IL-2.

FIG. 59 shows increases in the numbers of expanding cells by positively selected primary, non-osteoclast expanded and osteoclast-expanded T cells when compared to negatively selected NK cells or negatively selected T cells from healthy donors.

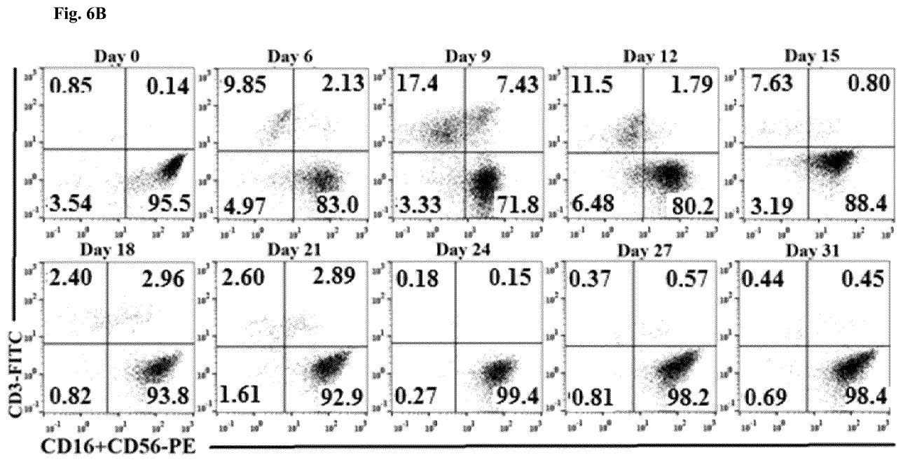

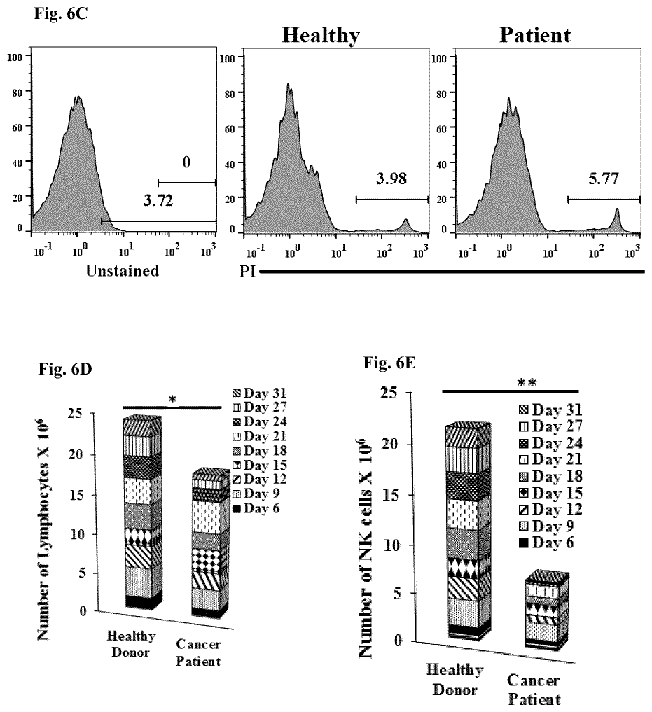

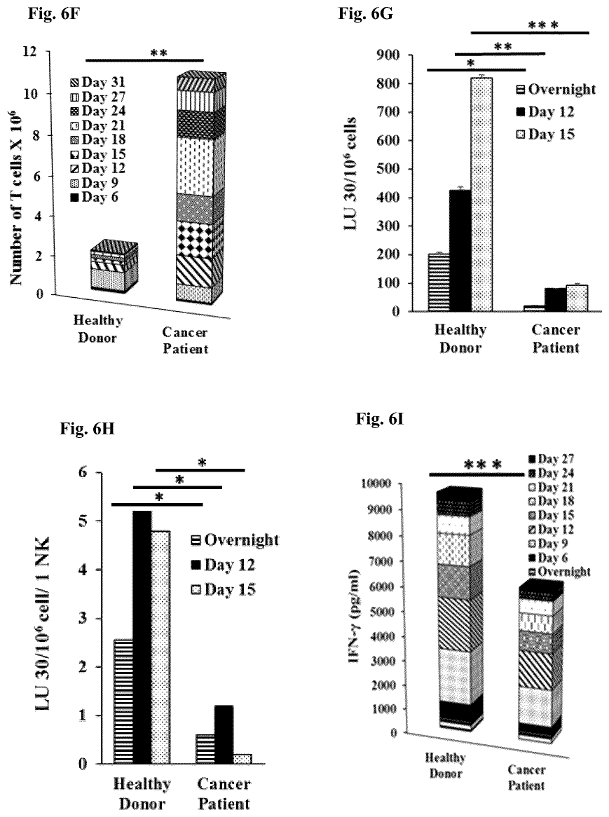

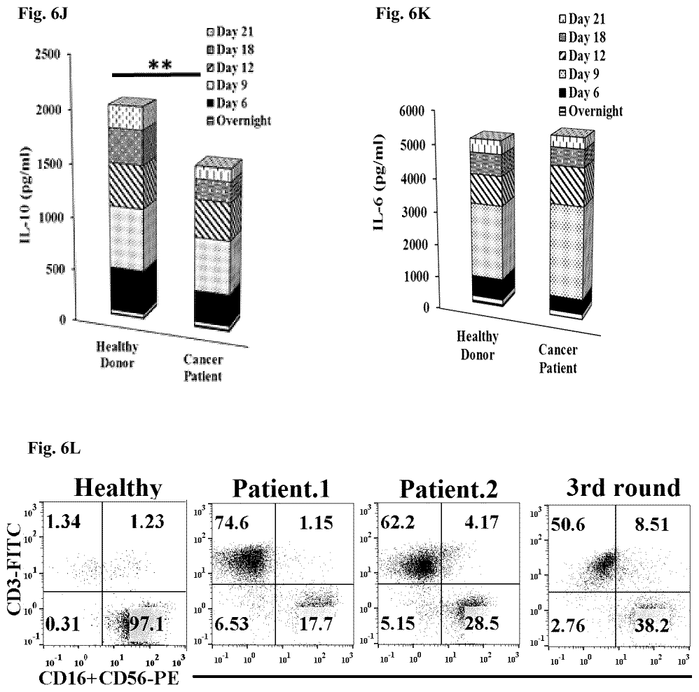

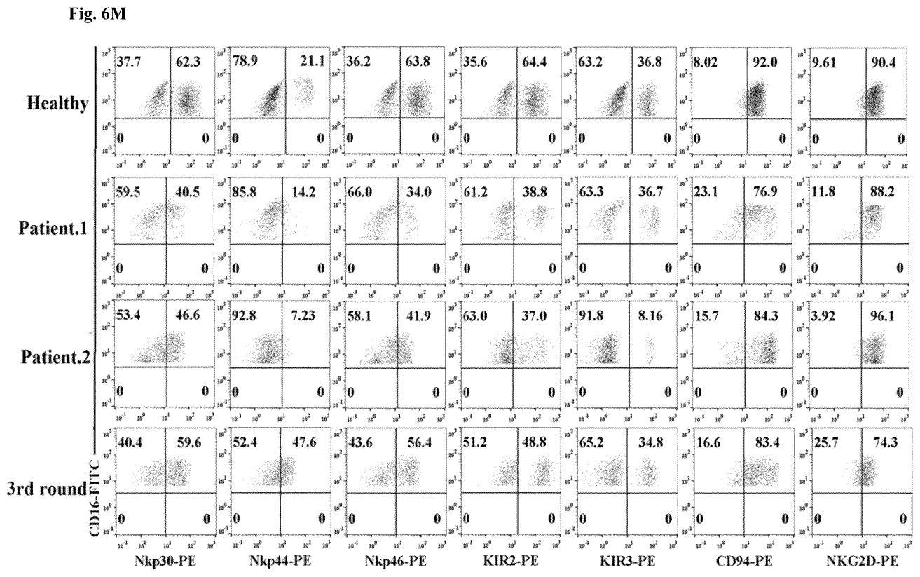

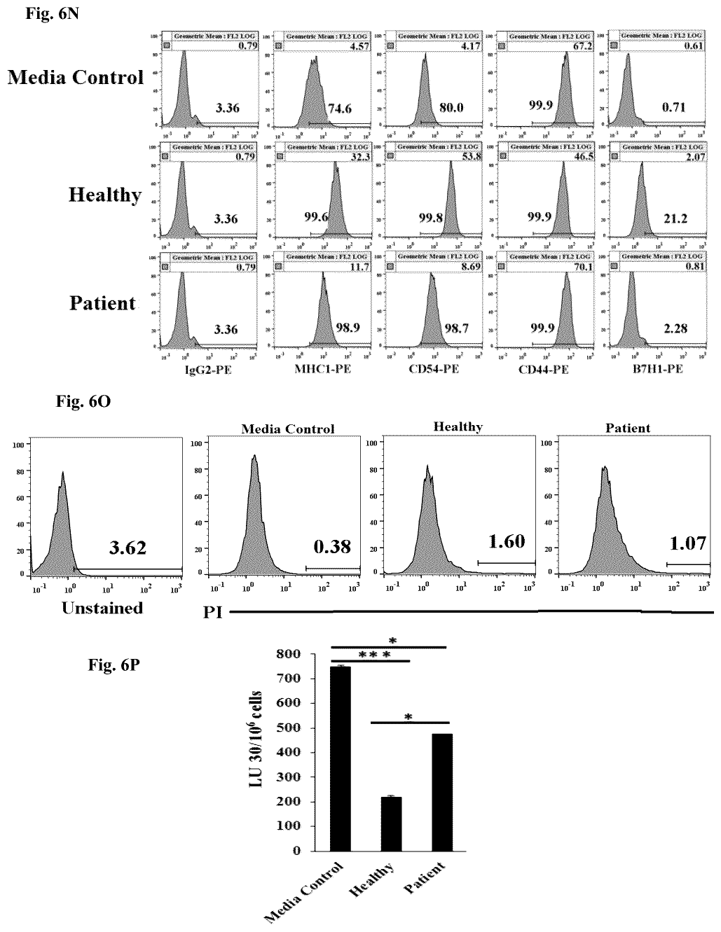

FIG. 6O shows decreases in the numbers of expanding cells by positively selected primary, non-osteoclast expanded and osteoclast-expanded patient's T cells when compared to those obtained from healthy donors.

FIG. 61 shows levels of cytokines and chemokines in sera from pancreatic patients' blood.

FIG. 62A-FIG. 62K show that single injection of super-charged NK-cells with/without feeding AJ2 inhibited tumor growth in hu-BLT mice. Hu-BLT mice were generated as described in Materials and Methods, and shown in figure (FIG. 62A). Hu-BLT and NSG mice were implanted orthotopically with 1×10⁶ human OSCSCs into the floor of the mouth, and after 7-10 days a group of hu-BLT mice were injected with 1.5×10⁶ super-charged NK cells through tail vein, and mice were monitored for disease progression. Another group of hu-BLT mice were fed with AJ2 probiotic bacteria (5 billion/day) every 48 hours 2 weeks prior to the implantation of OSCSCs and after implantation of the tumors in the presence and absence of NK injection until the experiments were terminated (FIG. 62B). Weight loss was monitored by weighing the mice on a weekly basis. One of 3 representative experiments is shown in this figure (FIG. 62C). Upon termination of the experiment, mice were sacrificed, and the pictures of tumors were taken after resection (FIG. 62D), and weighed (n=4) (FIG. 62E). Mice were implanted with human OSCSCs and injected with NK cells and fed with AJ2, as shown in FIG. 62B, and the tumors were resected and weighed post mortem (n=4) (FIG. 62F). PBMCs were isolated from hu-BLT mice and humans and surface expression of human CD3 (n=5) (FIG. 62G), CD4 (n=5) (FIG. 62H), CD8 (n=5) (FIG. 62I), CD19 (n=3) (FIG. 62J) and CD16 (n=5) (FIG. 62K) were determined within CD45+ immune cells using antibody staining followed by flow cytometric analysis.

FIG. 63A-FIG. 63I show that injection of super-charged NK-cells with/without feeding AJ2 restored and increased IFN-γ secretion and cytotoxic function of NK-cells in blood, spleen, BM, enriched-NK cells, and purified CD3+ T-cells in tumor-bearing hu-BLT mice. Hu-BLT mice were implanted with human OSCSCs and injected with NK cells, and fed AJ2 as described in FIG. 62B, and a week after NK cell injection mice were injected with anti-PD1 (50 μg/mice) via tail-vein injection. Following sacrifice, spleen (n=5) (FIG. 63A), BM (n=5) (FIG. 63B) and peripheral blood (n=5) (FIG. 63C) were collected, single cell suspensions were prepared from each tissue and (1×10⁶ cells/ml for spleen and BM and 0.7×10⁶ cells/ml for PBMCs) were treated with IL-2 (1000 U/ml) for 7 days. NK enriched cells were isolated from splenocytes, and (1×10⁶ cells/ml) were treated with IL-2 (1000 U/ml) for 7 days (n=3) (FIG. 63D). Cytotoxicity assays were performed using standard 4-hour ⁵¹Cr release assay against OSCSCs, and the LU 30/10⁶ cells were determined using inverse number of cells required to lyse 30% of OSCSCs×100. Splenocytes (n=5) (FIG. 63E), BM cells (n=5) (FIG. 63F), PBMCs (n=5) (FIG. 63G) at (1×10⁶ cells/ml for spleen and BM and 0.7×10⁶ cells/ml for PBMCs) were each treated with IL-2 (1000 U/ml), and positively selected CD3+ T cells (n=4) from the splenocytes at 1×10⁶ cells/ml were treated with IL-2 (100 U/ml) (FIG. 63H) for 7 days, after which the supernatants were harvested and the levels of IFN-γ were determined using specific ELISAs. Fold changes in IFN-γ secretion in each tissue from each group of mice were determined over those obtained from mice injected with OSCSCs alone (FIG. 63E-FIG. 63H). The tumor-bearing mice that received NK injection had the highest increase in IFN-γ secretion as compared to tumor-bearing mice in the absence of NK injection, or NK injected non-tumor bearing control mice (FIG. 63I).

FIG. 64A-FIG. 64B show single injection of super-charged NK cells with/without AJ2 feeding increased numbers of CD8+ T cells in hu-BLT mice. Hu-BLT mice were implanted with OSCSCs and injected with NK cells, and fed with AJ2, as described in FIG. 62B, and the percentages of human CD8+ T cells within BM cells (n=3) (FIG. 64A) and splenocytes (n=3) (FIG. 64B) were determined using antibody staining followed by flow cytometric analysis.

FIG. 65A-FIG. 65J show single injection of super-charged NK cells with/without AJ2 feeding in BLT mice mediated in vivo tumor differentiation, increased IFN-γ secretion and mobilized increased numbers of human immune cells to the tumors, and resulted in decreased ex-vivo tumor growth. Hu-BLT and NSG mice were implanted with OSCSCs and injected with NK cells, as described in FIG. 62B. Following sacrifice, oral tumors were harvested, and single cell suspensions were prepared and the same numbers of cells (total of 3×10⁶ cells at 1×10⁶ cells/ml) from each group were cultured at day 0. On day 10 supernatants were removed and attached tumor cells were counted, and for subsequent cultures the numbers in each group were adjusted to those obtained from NK injected mice since they expanded the least numbers of tumors. On days 10, 14, 19 and 20 the total numbers of ex-vivo expanding tumor cells were determined in each group. One of several representative experiments is shown in this figure (FIG. 65A). Hu-BLT mice were implanted with OSCSCs or in vitro NK-differentiated-OSCSCs (diff-OSCSCs), or NK-differentiated-OSCSCs treated with antibodies against IFN-γ and TNF-α to block differentiation, followed by NK injection in hu-BLT mice as described in FIG. 62B. Following sacrifice, oral tumors were dissociated, and single cells were prepared and cultured at (total of 3×10⁶ cells at 1×10⁶ cells/ml), and the numbers of expanding tumor cells were determined as described in FIG. 65A. One of several representative experiments is shown in this figure (FIG. 65B). Hu-BLT mice were implanted with OSCSCs or diff-OSCSCs, or diff-OSCSCs treated with antibodies against IFN-γ and TNF-α, and injected with NK cells and/or fed with AJ2 as described in FIG. 62B. Following sacrifice, oral tumors were harvested and cultured and the numbers of expanding tumor cells were determined as described in FIG. 65A (n=7) (FIG. 65C). Hu-BLT and NSG mice were implanted with OSCSCs, followed by NK injection in hu-BLT mice as described in FIG. 62A. Oral tumors were harvested, and single cell suspensions were prepared. The percentages of infiltrating hu-CD45C immune cells within the non-attached cells at day 12 of culture were determined using antibody staining followed by flow cytometric analysis. One of three representative experiments is shown in this figure (FIG. 65D). Oral tumors from hu-BLT and NSG mice were cultured as described in FIG. 4A and treated with IL-2 (1000 U/ml), and their supernatants were harvested on days shown in the figure and the levels of IFN-g were determined using ELISA. One of several representative experiments is shown in this figure (FIG. 65E). Expression of human CD54 and MHC-I were assessed on day 10 of oral tumor cultures from hu-BLT and NSG mice using flow cytometric analysis after staining with their respective antibodies. One of several representative experiments is shown in this figure (FIG. 65F). Purified NK cells (1×10⁶ cells/ml) from the peripheral blood of the healthy human donor were left untreated or treated with IL-2 (1000 U/ml) for 18 hours before they were added to ⁵¹Cr labeled OSCSCs cultured from the resected tumors of different experimental groups of hu-BLT mice, and compared it to the cultures of OSCSCs maintained in the lab at various effector to target ratios. NK cell-mediated cytotoxicity was determined using a standard 4-hour ⁵¹Cr release assay. LU30/10⁶ cells were determined as described in the Materials and Methods (n=4) (FIG. 65G and FIG. 65H). Oral tumor cells from hu-BLT mice as described in FIG. 65A were treated with IL-2 (1000 U/ml), and supernatants were harvested after 3 and 7 days and the levels of VEGF secretion were determined using specific ELISAs. The decrease in VEGF secretion by tumors obtained from NK-injected animals (n=6) were calculated based on the amounts obtained from OSCSCs alone injected mice (FIG. 65I). Infiltrating percentages of hu-CD45C immune cells within the oral tumors dissociated from different experimental groups of hu-BLT mice were determined using flow cytometric analysis after staining with antibody. One of several representative experiments is shown in this figure (FIG. 65J).

FIG. 66A-FIG. 66C show single injection of super charged NK cells with/without AJ2 feeding in tumor bearing mice restored and increased cytokine, chemokine and growth factor secretions within serum obtained from peripheral blood of hu-BLT mice. Serum from peripheral blood was obtained as described in Example 4 and multiplex arrays were performed to determine secretion of IFN-γ, one of the four representative experiments is shown here (FIG. 66A). Fold changes of IFN-γ were determined based on the values obtained from control hu-BLT mice (n=5) (FIG. 66B). Multiplex arrays were used to determine cytokines, chemokines, and growth factor secretion in sera obtained from the peripheral blood (FIG. 66C).

FIG. 67A-FIG. 67B show CDDP or Paclitaxel with and without NAC induce significant cell death in OSCSCs differentiated with NK-supernatants and not in poorly-differentiated tumors. Highly purified NK cells were treated with the combination of IL-2 (1000 U/mL) and anti-CD16mAb (3 mg/mL) for 18 hours, after which the NK supernatants were added to OSCSCs in the presence of anti-TNF-α (1:100) and anti-IFN-g (1:100) for a period of 5 days. Thereafter, OSCSCs were detached and treated with/without Cisplatin for 18-24 hours. The viability of OSCSCs was then determined using PI staining and flow cytometric analysis. One of 3 representative experiments is shown in this figure (FIG. 67A). OSCSCs were treated with the supernatants from NK cells as described in FIG. 67A. Afterwards, tumors were detached and treated with/without NAC (20 nM) for 24 hours, followed by treatment with Paclitaxel for 18-24 hours. OSCSCs viability was determined by PI staining and flow cytometric analysis. One of 3 representative experiments is shown in this figure (FIG. 67B).

FIG. 68A-FIG. 68E show monocytes or osteoclasts from tumor-bearing mice injected with NK-cells or implanted with only NK-differentiated OSCSC-tumors induced significantly more IFN-g from autologous or allogeneic NK-tumor co-cultures when compared to those of tumor-alone implanted mice with NK cells. Hu-BLT mice were implanted with OSCSCs and injected with NK cells, and fed AJ2 as described in FIG. 62B. After sacrifice, NK cells from splenocytes and monocytes from BM cells were isolated as described herein. Autologous NK cells were left untreated or treated with IL-2 (1000 U/ml) in combination with monocytes (NK:monocytes, 2:1) and at day 7 after the co-culture, NK cells were used as effector cells in a standard 4-hour ⁵¹Chromium release assay against OSCSCs. The LU 30/10⁶ cells were determined using inverse number of NK cells required to lyse 30% of the target cells×100 (FIG. 68A). Autologous NK cells were left untreated or treated with IL-2 (1000 U/ml) or with the combination of IL-2 (1000 U/ml) and LPS (100 ng/ml) in the absence and presence of monocytes (NK:monocytes, 2:1) for 7 days, after which the supernatants were harvested and IFN-g secretion were determined using single ELISA (FIG. 68B). OCs were generated from purified hu-BLT monocytes, as described in Example 4. Purified allogeneic NK cells from healthy human donors were pre-treated with IL-2 (1000 U/mL) and anti-CD16mAb (3 mg/mL) for 18 hours and then cultured with hu-BLT-OCs in the presence of sAJ2 (NK:OCs:sAJ2, 2:1:4). After culture, numbers of NK cells in the culture were counted on day 5, 8, 12 and 15 using microscopy (FIG. 68C). The supernatants were harvested from cultures on days 5, 8, 12 and 15 and the IFN-g secretion was determined using single ELISA (FIG. 68D). The levels of IFN-g obtained from ELISA were determined in 1×10⁶ cells using cell counts from FIG. 68E.

### DETAILED DESCRIPTION OF THE INVENTION

The present invention relates, in part, to a method of activating a NK cell in vitro or ex vivo, comprising culturing the NK cell in a medium comprising an osteoclast cell (OC). Similarly, a method is provided herein to activate a T cell in vitro or ex vivo, comprising culturing the T cell in a medium comprising a dendritic cell (DC). The present invention further provides a method of activating a NK cell relative to a T cell in vitro or ex vivo, comprising culturing the NK cell and the T cell in a medium comprising an osteoclast (OC). The present invention further provides a method of activating a T cell relative to a NK cell in vitro or ex vivo, comprising culturing the NK cell and the T cell in a medium comprising a dentritic cell (DC). Such activated NK cells may be used to improve host immune responses and may be used to treat diseases (e.g., cancers). In some aspects, the present invention provides a method of activing NK cells in vivo, optionally activing NK cells relative to T cells, by osteoclast cells (OCs). In some embodiments, OCs or OC culture supernatant may be administered to a subject to treat a disease (e.g., cancer). In some embodiments, probiotic bacteria (e.g., sAJ2) may be added to improve the function of OCs to activate NK cells. Besides osteoclast cells (OCs) and dentritic cells (DCs), other agents capable of activating NK cells or T cells may be also added to the cell culture or administered to a subject, including any genes, proteins, metabolites, and the like.

### I. Definitions

The articles “a” and “an” are used herein to refer to one or to more than one (i.e. to at least one) of the grammatical object of the article. By way of example, “an” element means one element or more than one element.

The term “administering” is intended to include routes of administration which allow an agent (e.g., at least one osteoclast cell (OC) or dendritic cell (DC), a cell culture comprising at least one osteoclast cell (OC) or dendritic cell (DC), the supernatant of such cell culture, any compositions comprising such OC(s) or DC(s), at least one probiotic bacterial, any compositions comprising such probiotic bacteria, other agents capable of activating NK cells and/or T cells or facilitating the function of such OC(s) or DC(s) and/or probiotic bacteria, etc., also including processed (i.e., isolated, purified, concentrated, or after other processes for therapeutic or other uses) forms of various agents described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “activating” or “activation” refers to an enhancement of the function of a target. For example, the instant disclosure provides a method of activating a NK cell or a T cell in vitro, ex vivo, and/or in vivo, optionally wherein such activation is preferential relative to T cells or NK cells, respectively. In the instant disclosure, the activation of a cell refers to an enhancement of the function of such cell, including at least an enhancement of activity and/or at least one cellular function (e.g., cytotoxicity, cell division and/or growth rate, etc.) of each cell of a cell type (e.g., NK cells or T cells), an enhancement of cell numbers (e.g., cell expansion) of a cell type, or both. In some embodiments, the agent used herein activates at least one cell, such as NK cell(s) or T cell(s). In other embodiments, the agent used herein preferentially activates one cell type (e.g., NK cells, or T cells) relative to another (e.g., T cells, or NK cells).

The term “enhancement” is used interchangeably with terms “increase,” “upregulation,” “improvement,” or the like in the instant disclosure, referring to any increase in amount meaningful for the function of an agent and/or a target. For example, an enhancement of activity and/or cell numbers of a cell type (e.g., NK cells or T cells) may be “significant,” when the increase in amount is greater than the original amount and/or the normal amount in a control (e.g., the activity and/or cell number of a cell type in a normal subject or a subject without a disease or disorder (e.g., a cancer)) by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or more than that amount. Alternately, the enhancement of activity and/or cell numbers of a cell type can be considered “significant” if such enhancement is at least about two, and preferably at least about three, four, or five times, or more, than the original activity/amount and/or the normal activity/amount. Such “significance” can also be applied to any other measured parameter described herein, such as for expression, inhibition, cytotoxicity, cell growth, and the like. In some embodiments, an enhancement of activity and/or cell numbers of a cell type (e.g., NK cells or T cells) may not be “significant” as described herein but still be an increase sufficient for a skilled artisan to understand its biological relevance.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. In some embodiments, such characteristics include at least one of silencing, decreasing, and/or avoiding host immune response, and/or being resistant to host cell (e.g., NK cells) lysis and/or differentiations. In some embodiments, such cancer-causing cells are cancer stem cells (e.g, oral squamous carcinoma stem cells (OSCSCs)). In some embodiments, such cells exhibit such characteristics in part or in full due to at least one genetic mutations. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is oral cancer, breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In some embodiments, the cancer is “triple negative breast cancer” or “TNBC,” which refers to breast cancers that are estrogen receptor (ER) negative, progesterone receptor (PR) negative, and human epidermal growth factor receptor 2 (HER-2) negative (Pegram et al. (1998) J. Clin. Oncol. 16:2659-2671; Wiggans et al. (1979) Cancer Chemother. Pharmacol. 3:45-48; Carey et al. (2007) Clin. Cancer Res. 13:2329-2334).

In certain embodiments, the cancer is a “PI3Kbeta-dependent cancer,” which can refer to a cancer that is functionally dependent on PI3Kbeta. For instance, even if the expression level of PI3Kbeta (e.g., PI3Kbeta mRNA, PI3Kbeta protein, newly synthesized PI3Kbeta protein, etc.) in a tumor tissue is comparable to its expression level in normal tissue, a cancer is PI3Kbeta-dependent if inhibition of the PI3Kbeta mRNA and/or protein, directly or indirectly such as by using RNAi or any other means, or deletion of the PI3Kbeta gene (e.g., by knock-out or clustered regularly interspaced short palindromic repeats (CRISPR) technology) leads to inhibition of oncogenesis, tumor cell proliferation, tumor metastasis or induces tumor cell differentiation. The term “PI3Kbeta-dependent cancer” also refers to a cancer in which PI3Kbeta is expressed (e.g., PI3Kbeta mRNA, PI3Kbeta protein, newly synthesized PI3Kbeta protein, etc.) at a significantly higher level than the normal amount of PI3Kbeta expressed in a non-cancerous cell of the same cell type as the PI3Kbeta-dependent cancer.

The term “micrometastasis” as used herein is preferably defined as a group of confluent cancer cells measuring from greater than 0.2 mm and/or having greater than 200 cells to 2 mm in maximum width. More preferably “micrometastasis” is defined as a group of confluent cancer cells from 0.2 mm to 2 mm in maximum width (see Edge et al. (2010) AJCC Cancer Staging Manual and Handbook (7th ed.)). An alternative preferred definition of “micrometastasis” is a confluent group of at least 1000 cancer cells and at least 0.1 mm in widest dimension up to 1 mm in widest dimension. Micrometastasis is generally not visible in standard contrast MRI imaging or other clinical imaging techniques. However, in certain cancers, radioactive antibodies directed to tumor selective antigens (e.g., Her2 for breast cancer metastasis) allows for visualization of micrometastasis. Other indirect detection methods include contrast media leakage at brain micrometastasis sites due to VEGF induced vascular leakage (Yano et al. (2000) Cancer Res. 60:4959-49067; U.S. Pat. Publ. 2015/0352113). More sensitive imaging techniques may also be applied to detect micrometastases. For example, blood volume may be imaged by MRI using the alternative contrast agent, USPIO (Molday Iron, Biopal, Worcester, Mass.) to detect micrometastasis (Yin et al. (2009) Clin. Exp. Metastasis. 26:403-414).

The term “control” refers to any reference standard suitable to provide a comparison to the expression products, cell numbers, and/or cellular functions in the test sample. In certain embodiments, the control comprises obtaining a “control sample” from which expression product levels, cell numbers, and/or cellular functions are detected and compared to the expression product levels, cell numbers, and/or cellular functions from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In other preferred embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels, cell numbers, and/or cellular functions can be used in combination as controls in the methods of the present invention. In certain embodiments, the control may comprise normal or non-cancerous cell/tissue sample. In other preferred embodiments, the control may comprise an expression level, numbers of a certain cell type (e.g., NK cells or T cells), and/or a cellular function of a certain cell type for a set of patients, such as a set of cancer patients, or for a set of cancer patients receiving a certain treatment, or for a set of patients with one outcome versus another outcome. In the former case, the specific expression product level, cell numbers, and/or cellular functions of each patient can be assigned to a percentile level of expression, cell numbers, and/or cellular functions, or expressed as either higher or lower than the mean or average of the reference standard expression level, cell numbers, and/or cellular functions. In other preferred embodiments, the control may comprise normal cells, cells from patients treated with combination chemotherapy, and cells from patients having benign cancer. In other embodiments, the control may also comprise a measured value for example, average level of expression of a particular gene, cell numbers and/or cellular functions of a particular cell type (e.g, NK cells or T cells) in a population compared to the level of expression of a housekeeping gene or another cell type in the same population. Such a population may comprise normal subjects, cancer patients who have not undergone any treatment (i.e., treatment naive), cancer patients undergoing standard of care therapy, or patients having benign cancer. As demonstrated by the data below, the methods of the present invention are not limited to use of a specific cut-point in comparing the level of expression product, cell numbers, and/or cellular functions in the test sample to the control.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “cytokine” refers to a broad and loose category of small proteins (˜5-20 kDa) that are important in cell signaling. Their release has an effect on the behaviour of cells around them. cytokines are involved in autocrine signaling, paracrine signaling and endocrine signaling as immunomodulating agents. Cytokines include chemokines, interferons, interleukins, lymphokines, and tumour necrosis factors, and may additionally include hormones or growth factors in the instant disclosure. Cytokines are produced by a broad range of cells, including immune cells like macrophages, B lymphocytes, T lymphocytes and mast cells, as well as endothelial cells, fibroblasts, and various stromal cells. Preferred cytokines are exemplified in the specification and the Figures of the instant disclosure, for example, in Table 1.

The term “cytokine/chemokine activity,” includes the ability of a cytokine or a chemokine to modulate at least on of cellular functions. Generally, cytokines or chemokines modulate the balance between humoral and cell-based immune responses, and they regulate the maturation, growth, and responsiveness of particular cell populations. Thus, the term “cytokine/chemokine activity” includes the ability of a cytokine or chemokine to bind its natural cellular receptor(s), the ability to modulate cellular signals, and the ability to modulate the immune response.

The term “immune response” includes T cell mediated and/or B cell mediated immune responses. Exemplary immune responses include T cell responses, e.g., cytokine production and cellular cytotoxicity. In addition, the term immune response includes immune responses that are indirectly affected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state. For example, kinase activity of a mutant PI3 kinase or a PI3 kinase that is contacted with a PI3 kinase inhibitor is inhibited if the kinase activity is decreased due to the mutation and/or contact with the inhibitor, in comparison to the wild-type PI3 kinase and/or the PI3 kinase not contacted with the inhibitor. Such inhibition can be induced, such as by application of agent at a particular time and/or place, or can be constitutive, such as by a heritable mutation. Such inhibition can also be partial or complete (e.g., essentially no measurable activity in comparison to a reference state, such as a control like a wild-type state). Essentially complete inhibition is referred to as blocked.

The term “interaction”, when referring to an interaction between two molecules, refers to the physical contact (e.g., binding) of the molecules with one another. Generally, such an interaction results in an activity (which produces a biological effect) of one or both of said molecules.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression and/or activity of a biomarker is the level of expression and/or activity of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. The same determination can be made to determine overactivity or underactivity.

### NK Cells

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

NK cells are negatively regulated by major histocompatibility complex (MHC) class I-specific inhibitory receptors (Karre et al., 1986; Ohlen et al, 1989). These specific receptors bind to polymorphic determinants of MHC class I molecules or HLA present on other cells and inhibit NK cell lysis. In humans, certain members of a family of receptors termed killer Ig-like receptors (KIRs) recognize groups of HLA class I alleles.

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an immunostimulatory motif (ITAM). Other KIR gene products are inhibitory in nature.

### Probiotic Bacteria

In some embodiments, the instant invention is drawn to a composition comprising at least one probiotic bacterial strain, capable of regulating NK cell function. Such probiotic bacteria induce significant split anergy in activated NK cells, leading to a significant induction of IFN-γ and TNF-α. In addition, such probiotic bacteria induce significant expansion of NK cells.

Many commercial probiotics are available, having various effects of reducting gastrointestinal discomfort or strengthening of the immune system. Preferred probiotic bacteria species for use in the compositions and methods described herein include those commercially available strains of probiotic bacteria (such as sAJ2 bacteria), especially those from the Streptococcus (e.g., S. thermophiles), Bifidobacterium (e.g., B. longum, B. breve, B. infantis, B. breve, B. infantis), and Lactobacillus genera (e.g., L. acidophilus, L. helveticus, L. bulgaricus, L. rhamnosus, L. plantarum, and L. casei). The instant disclosure comprising methods of administering at least one probiotic bacterial strain, preferably a combination of two or more different bacterial strains, to a subject, preferably a mammal (e.g., a human). Such administration may be systemically or locally (e.g., directly to intestines) performed. A preferably administration route is oral administration. Other routes (e.g., rectal) may be also used. For administration, either the bacteria (e.g., in a wet, sonicated, grounded, or dried form or formula), the bacterial culture medium containing the bacteria, or the bacterial culture medium supernatant (not containing the bacteria), may be administered.

### Osteoclasts

Osteoclasts are a type of bone cell, derived from hematopoietic stem cells. Their function, resorbing bone tissue, is critical for the maintenance, repair, and remodeling of bones. Bone homeostasis is achieved when there is a balance between osteoblast bone formation and osteoclast bone resorption. Osteoclasts mature through stimulation from osteoblasts expressing RANKL, and their interaction, mediated by firm adhesion via ICAM-1. Osteoclasts also express many ligands for receptors present on activated NK cells. They reported that osteoclasts express ULBP-1, ULBP-2/5/6 and ULBP-3, but little or no MIC-A, MIC-B, or MHC class I-like ligands for NKG2D, the activating receptor of NK cells.

Osteoclasts (OCs), in comparison to dendritic cells (DCs) and monocytes, are significant activators of NK cell expansion and function (Tseng et al. (2015) Oncotarget 6(24):20002-25). Additionally, osteoclasts secrete significant amounts of IL-12, IL-15, IFN-γ and IL-18, which are known to activate NK cells; osteoclasts also express important NK-activating ligands. The instant disclosure provides a novel strategy on how to expand highly functional, super-charged, osteoclast-expanded NK cells to levels that are significantly higher than those established by other methodologies. Several in vitro NK expansion techniques have been developed to establish a higher therapeutic cell dosage, while boosting activity and in vivo proliferative potential of NK cells. Some of these techniques include the stimulation of peripheral blood mononuclear cells (PBMCs), PBMC-purified populations of NK cells, or the use of human cord blood, sometimes in combination with various feeder cells such as K562 cells expressing membrane-bound IL-15 and 41BB ligand (K562-mb15-41BBL), EBV-TM-LCL, Wilm's tumor or irradiated PBMCs. These studies have generated clinically relevant NK cell numbers that have good function.

### Dendritic Cells

Dendritic cells (DCs) are antigen-presenting cells (also known as accessory cells) of the mammalian immune system. Their main function is to process antigen material and present it on the cell surface to the T cells of the immune system. They act as messengers between the innate and the adaptive immune systems.

Dendritic cells are present in those tissues that are in contact with the external environment, such as the skin (where there is a specialized dendritic cell type called the Langerhans cell) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites that give the cell its name. While similar in appearance, these are structures distinct from the dendrites of neurons. Immature dendritic cells are also called veiled cells, as they possess large cytoplasmic “veils” rather than dendrites The instant disclosure provides a novel method to activate NK cells using osteoclasts, resulting in enhanced sensitization of tumor target cells to NK cell-mediated apoptosis, as well as cytokine production. The term “activate”, “activation,” or “activating” refers to enhance the expansion of NK cells and/or activating NK cell functions, either alone or in combination. The term “NK cell function(s)” refers to any function of NK cells, such as cytotoxicity and/or cytokine/chemokine production/secretion activities.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “response to anti-cancer therapy” or “response to a therapy with a composition comprising at least one of probiotic bacteria, alone or in combination with other NK immunotherapies” relates to any response of the hyperproliferative disorder (e.g., cancer) to an anti-cancer agent(s) such as treatment with a composition comprising at least one of probiotic bacteria, alone or in combination with other NK immunotherapies, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, such as in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit a favorable response (i.e., will exhibit a lack of response or be non-responsive).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., by treating with the compositions described herein). In some embodiments, normal cells are not affected to an extent that causes the normal cells to be unduly injured. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164), cell death assays (Weisenthal et al. (1984) Cancer Res. 94:161-173; Weisenthal et al. (1985) Cancer Treat. Rep. 69:615-632; Weisenthal L M, In: Kaspers et al. eds. Drug Resistance in Leukemia and Lymphoma. Langhorne, P A: Harwood Academic Publishers, 1993: 415-432; Weisenthal et al. (1994) Contrib. Gynecol. Obstet. 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “specific binding” refers to an agent, such as an antibody, binding to a pre-determined target, such as an antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “synergistic effect” refers to the combined effect of two or more anti-cancer agents (e.g., treatment with a combination of a composition comprising at least one of probiotic bacteria, alone or in combination with other NK immunotherapies) can be greater than the sum of the separate effects of the anti-cancer agents alone.

The term “subject” refers to any healthy animal, mammal or human, or any animal, mammal or human afflicted with a cancer, e.g., brain metastasis, lung, ovarian, pancreatic, liver, breast, prostate, colon carcinomas, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human. In other embodiments, the subject is an animal model of a cancer. For example, the animal model can be an orthotopic xenograft animal model of human oral squamous carcinoma, or comprising cancer stem cells (CSCs)/undifferentiated tumors.

In other embodiments of the methods of the present invention, the subject has not undergone treatment, such as chemotherapy, radiation therapy, targeted therapy, and/or anti-immune therapy (such as NK cell-related immunotherapies). In still other embodiments, the subject has undergone treatment, such as chemotherapy, radiation therapy, targeted therapy, and/or anti-immune therapy (such as NK cell-related immunotherapies).

In certain embodiments, the subject has had surgery to remove cancerous or pre-cancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to treat and/or determine the responsiveness to a composition comprising at least one of probiotic bacteria, alone or in combination with other NK immunotherapies, of many different cancers in subjects such as those described herein.

### III. Anti-Cancer Therapies

In one aspect, other anti-cancer therapies and/or immunotherapies combination or combinations of therapies (e.g., one or more PI3Kbeta-selective inhibitors, such as KIN193, in combination with one or more immune checkpoint inhibitors, such as an anti-PD-1 antibody, either alone or in combination with yet additional anti-cancer therapies, such as targeted therapy) can be administered, particularly if a subject has first been indicated as being a likely responder to a composition as disclosed herein. In other embodiments, such therapy can be avoided once a subject is indicated as not being a likely responder to such therapy and an alternative treatment regimen, such as targeted and/or untargeted anti-cancer therapies can be administered together with the composition as disclosed herein.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with a therapy as disclosed herein. As described below, agents can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In other embodiments, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

The term “targeted therapy” refers to administration of agents that selectively interact with a chosen biomolecule to thereby treat cancer. One example includes breast or ovarian cancer antigens.

Alternatively, immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In certain embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In yet other embodiments, surgical intervention can physically remove cancerous cells and/or tissues.

In still other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet other embodiments, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still other embodiments, photodynamic therapy (also called PDT, photoradiation therapy, phototherapy, or photochemotherapy) is used for the treatment of some types of cancer.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

The duration and/or dose of treatment with therapies may vary according to the particular therapeutic agent or combination thereof Δn appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

Clinical efficacy can be measured by any method known in the art. For example, the response to a therapy, such as a composition as disclosed herein, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular therapeutic regimen is at least 25%, 30%, 35%, 40%, 45%, 50%, 55%, 60%, 65%, 70%, 75%, 80%, 85%, or more.

Additional criteria for evaluating the response to a therapy as disclosed herein are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any composition as disclosed herein. The outcome measurement may be pathologic response to therapy given in the neoadjuvant setting. Alternatively, outcome measures, such as overall survival and disease-free survival can be monitored over a period of time for subjects following a therapy for whom biomarker measurement values are known. In certain embodiments, the same doses of a therapeutic composition are administered to each subject. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a therapy as disclosed herein can be determined using methods such as those described in the Examples section.

### 3. Pharmaceutical Compositions

The present invention provides pharmaceutically acceptable compositions of the compositions disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles.

Compositions described herein, e.g., compositions of probiotic bacteria, may be used for oral administration to the gastrointestinal tract, directed at the objective of introducing the probiotic bacteria to tissues of the gastrointestinal tract. The formulation for a therapeutic composition of the present invention may also include other probiotic agents or nutrients which promote spore germination and/or bacterial growth. An exemplary material is a bifidogenic oligosaccharide, which promotes the growth of beneficial probiotic bacteria. In certain embodiment, the probiotic bacterial strain is combined with a therapeutically-effective dose of an (preferably, broad spectrum) antibiotic, or an anti-fungal agent. In some embodiments, the compositions described herein are encapsulated into an enterically-coated, time-released capsule or tablet. The enteric coating allows the capsule/tablet to remain intact (i.e., undisolved) as it passes through the gastrointestinal tract, until after a certain time and/or until it reaches a certain part of the GI tract (e.g., the small intestine). The time-released component prevents the “release” of the probiotic bacterial strain in the compositions described herein for a pre-determined time period.

The therapeutic compositions of the present invention may also include known antioxidants, buffering agents, and other agents such as coloring agents, flavorings, vitamins or minerals.

In some embodiments, the therapeutic compositions of the present invention, e.g., osteoclasts, a cell culture of osteoclasts, and/or the supernatant of a cell culture of osteoclasts, can be administered alone, or in combined with a carrier which is physiologically compatible to the species to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. A preferred carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607. The osteoclasts, a cell culture of osteoclasts, and/or the supernatant of a cell culture of osteoclasts of the present disclosure can be administered using local and/or systemic administration routes known in the ar and described herein.

The osteoclast cells (OCs) or dendritic cells (DCs) described herein may be used for administration to the subject in any pharmaceutically acceptable composition through any administration route known in the art. For example, OCs or DCs, cell cultures comprising such OCs or DCs, or supernatants of such cell cultures, optionally with additional agent(s), may be administered in a pharmaceutical composition through systemic and/or local (e.g., to or near the cancer or tumor tissue) injections (e.g., intravenously).

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more bacterial strains as disclosed herein.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXAMPLES

### Example 1: Materials and Methods for Examples 2 and 3

### Cell Lines, Reagents, and Antibodies

RPMI 1640 complete medium with 10% fetal bovine serum (FBS) (Gemini Bio-Product) was used for cell cultures. Oral squamous carcinoma cells (OSCCs) and oral squamous carcinoma stem cells (OSCSCs) were isolated from cancer patients with tongue tumors at UCLA [see references 2 and 33-35; all citations below refer to the same reference list]. Alpha-MEM (Life Technologies, CA) with 10% FBS was used for osteoclast and DC cultures. M-CSF (Biolegend, CA) and RANKL, GM-CSF and IL-4 were purchased from PeproTech (NJ) and rh-IL-2 was obtained from NIH-BRB. Human CD3/CD28 T cell activator was purchased from stem cell technologies.

Antibodies for MHC-I, KIR2, KIR3, CD44, CD54, B7H1, CD16, NKG2D, MICA/B, KLGR1, CD45, CD3/16/56, CD8, CD3, CD28,CD4, GL3, NKp40, NKp30, NKp44, NKp46 and CD94 were purchased from Biolegend (San Diego, CA). ULBP 1-6 antibodies were purchased from R&D Systems. Propidium iodide (PI) was purchased from Sigma (St. Louis, MO). sAJ2 was prepared as described previously [36].

Purification of NK Cells and T Cells from Human PBMCs and Hu-BLT Splenocytes

NK cells and T cells were purified as described previously [37]. T cells from hu-BLT splenocytes were positively purified using isolation kits from Stem Cell Technologies (Stem Cell Technologies, Vancouver, Canada).

Purification of Monocytes and Generation of Dendritic Cells and Osteoclasts from Hu-BLT Mice and Human PBMCs

Written informed consent approved by UCLA Institutional Review Board (IRB) was obtained from healthy blood donors and all procedures were approved by the UCLA-IRB. Monocytes were purified as described previously [37]. Monocytes from hu-BLT mice were positively isolated from bone marrow using human CD14 isolation kit (eBioscience, San Diego, CA). Greater than 95% purity was achieved for each subset based on flow cytometric analysis. Monocytes were differentiated to osteoclasts by treating with M-CSF (25 ng/mL) and RANKL (25 ng/mL) for 21 days. To obtain DCs, monocytes were treated with GM-CSF (150 ng/mL) and IL-4 (50 ng/mL) for 7 days.

### Expansion of NK Cells

Human purified and hu-BLT enriched NK cells were activated with rh-IL-2 (1000 U/ml) and anti-CD16 mAb (3 μg/ml) for 18-20 hours before they were co-cultured with feeder cells and sAJ2. The culture medium with IL-2 was refreshed every three days.

Tumor Implantation and Tissue Preparation from Hu-BLT Mice

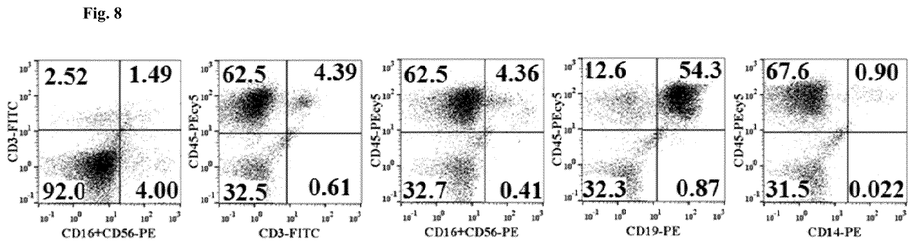

Animal research described in this manuscript was performed under the written approval of the UCLA Animal Research Committee (ARC) in accordance to all federal, state, and local guidelines. Combined immunodeficient NOD.CB17-Prkdcscid/J and NOD.Cg-Prkdcscid Il2rgtm1Wjl/SzJ (NSG lacking T, B, and natural killer cells) were purchased from Jackson Laboratory. Humanized-BLT (hu-BLT; human bone marrow/liver/thymus) mice were prepared on NSG background as described previously and in FIG. 8 [38, 39]. To establish orthotopic tumors, mice were first anesthetized with isoflurane in combination with oxygen, and tumor cells were then directly injected in the floor of mouth in suspension with 10 μl HC Matrigel (Corning, NY, USA) (1×10⁶ cells). Four to five weeks after the tumor injections, mice were euthanized and bone marrow, spleen, and blood were harvested and single cell suspensions were prepared [40].

### ELISA and Multiplex Cytokine Array Kit

Single ELISAs and multiplex assays were performed as described previously [37].

Cancer Stem Cell Differentiation with NK Cell Supernatants

Supernatants from NK cells were prepared and used for differentiation of OSCSCs as described previously [36]. Day 13 supernatants from OC expanded NK cells were used for differentiation.

### Surface Staining and Cell Death Assays

Staining was performed by labeling the cells with antibodies or propidium iodide as described previously [37, 41, 42]. Flow cytometry analysis was performed using Beckman Coulter Epics XL cytometer (Brea, CA) and results were analyzed using FlowJo vX software (Ashland, OR).

### ⁵¹Cr Release Cytotoxicity Assay

The ⁵¹Cr release assay was performed as described previously [43].

### Statistical Analysis

An unpaired or paired, two-tailed student t-test was performed for the statistical analysis. One-way ANOVA with a Bonferroni post-test was used to compare the different groups. ***(p value <0.001), **(p value 0.001-0.01), *(p value 0.01-0.05).

### Example 2: Osteoclast Activated Super-Charged NK Cells Preferentially and Rapidly Expand CD8+ T Cells Resulting in a Decline in Natural Killer Cell Numbers from Cancer Patients and BLT Humanized Mice

### Preferential Expansion and Significant Gain in Function of NK Cells by Osteoclasts and T Cells by Dendritic Cells

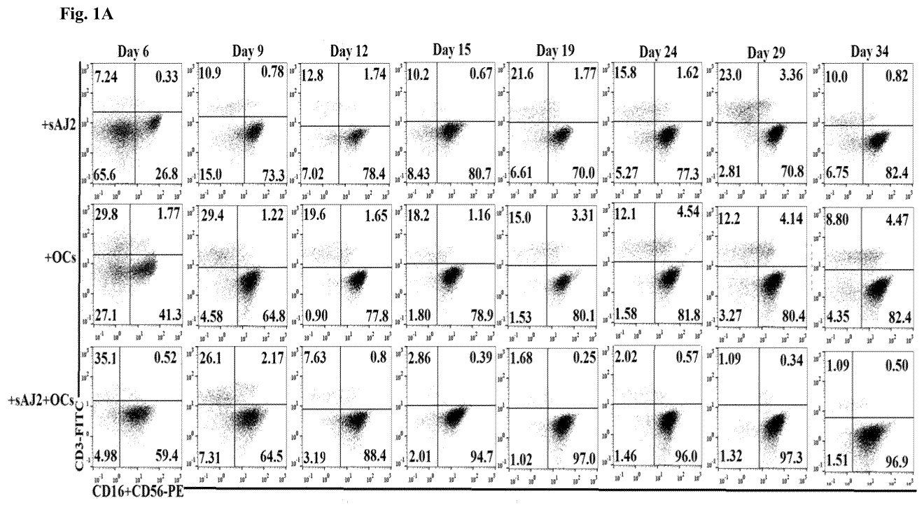

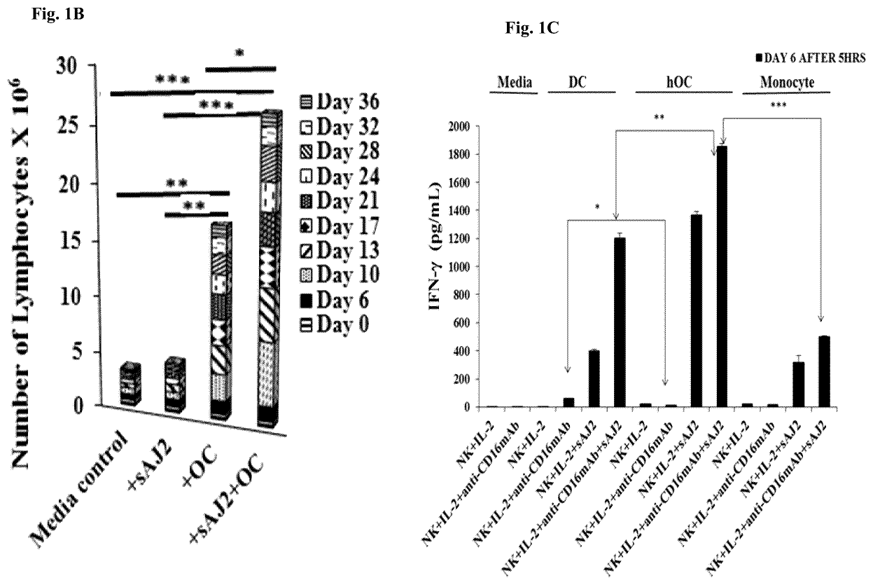

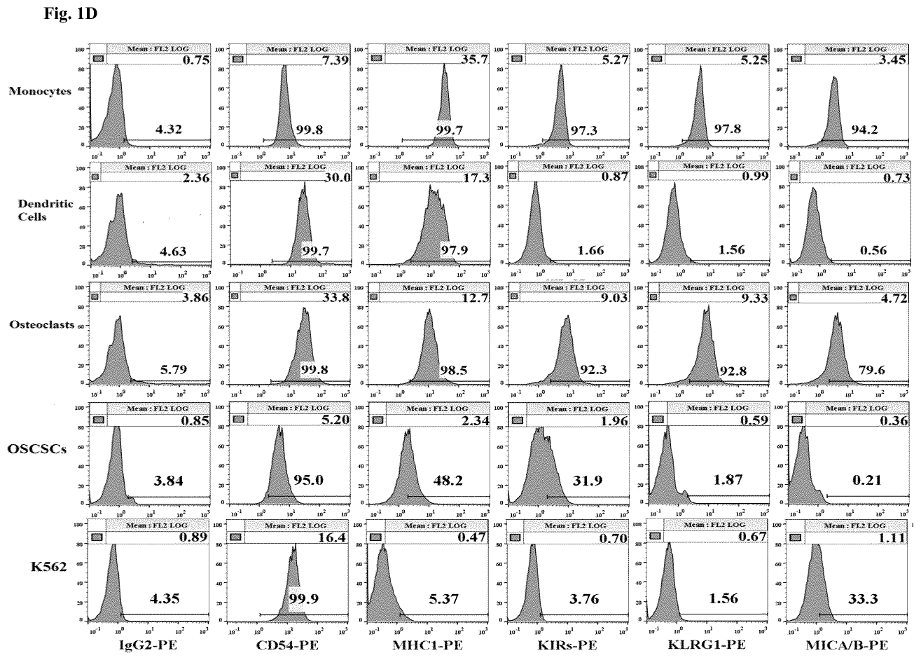

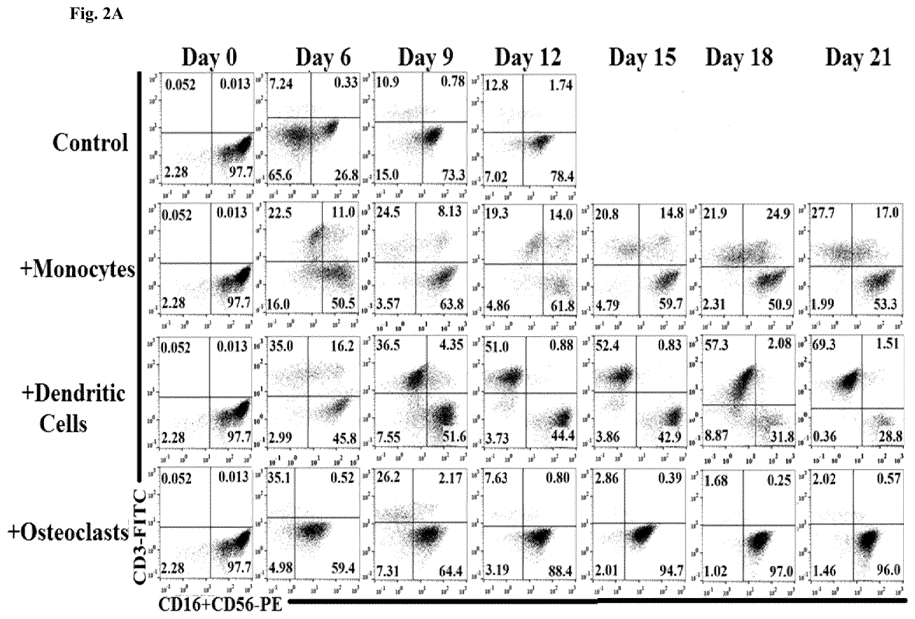

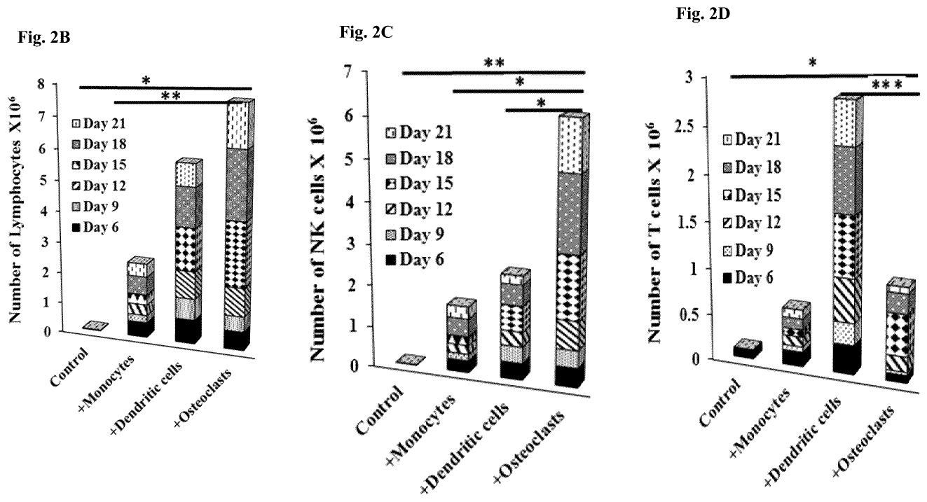

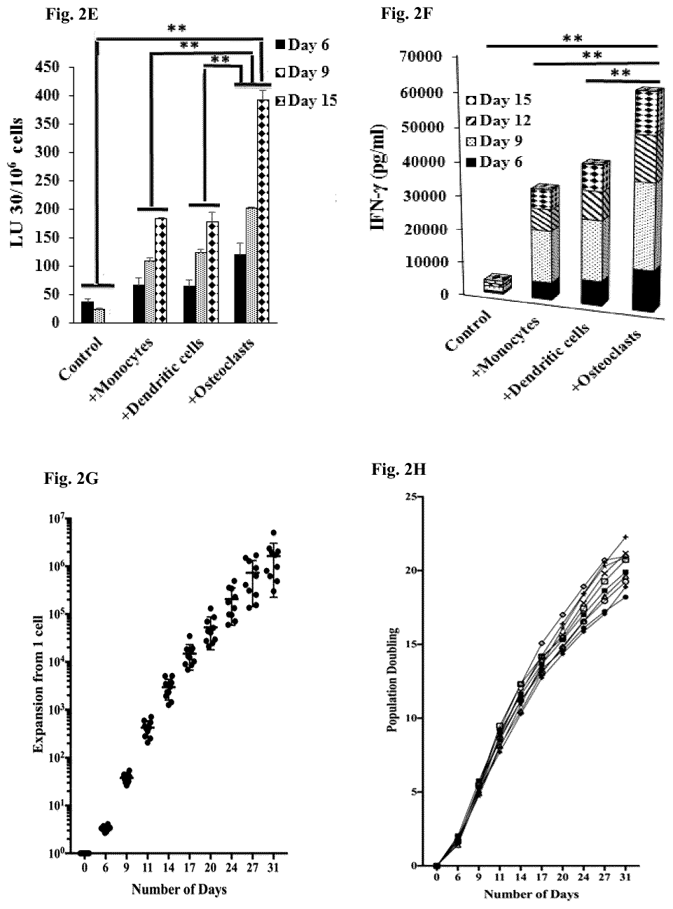

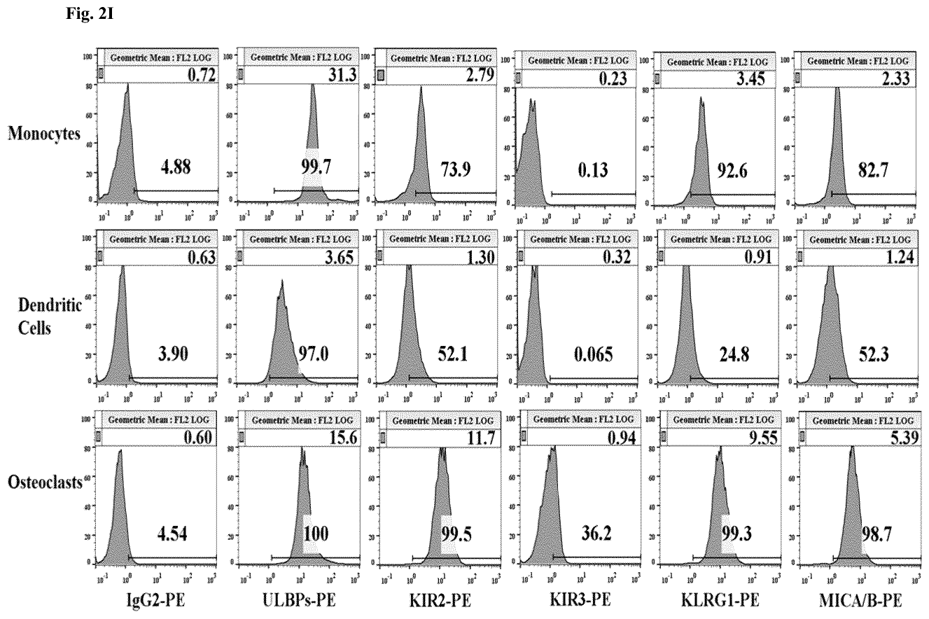

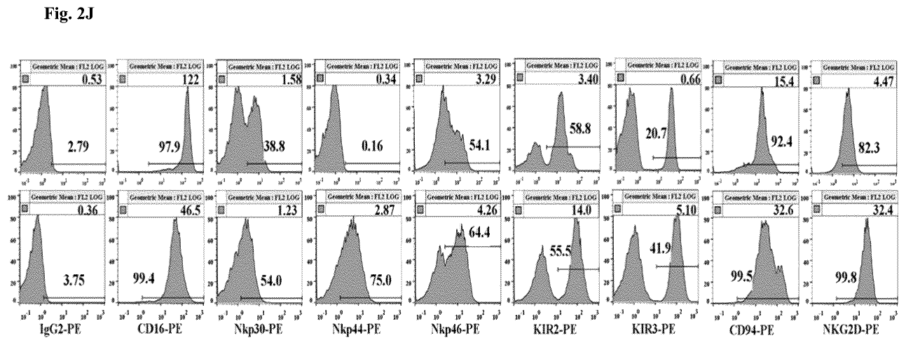

The activating effect of osteoclasts, monocytes and DCs on NK cell expansion and function was compared. NK cells were activated with IL-2 and anti-CD16 mAb 18-20 hours before their co-culture with OCs and/or sAJ2. The combination of OCs and sAJ2 preferentially expanded NK cells while maintaining a low proportion of T cells (FIG. 1). The rate of expansion and the levels of contaminating T cells in NK cultures were then compared between the co-cultures with OCs, DCs, and monocytes treated with sAJ2. NK cells co-cultured with OCs preferentially expanded NK cells and the rate of contaminating T cells remained very low throughout the first one to two months of the culture (FIGS. 2 and 4A). In contrast, DCs preferentially expanded T cells and the proportion of NK cells remained low. Although monocytes were also able to expand T cells, T cell expansion remained lower than those of NK cells. There was an initial increase in the proportion of T cells in all three cultures, however, in subsequent cultures, the rate of T cell expansion decreased in the co-cultures with OCs, whereas T cells continued expanding, and substantially increased in cultures with DCs. A steady state of T cell expansion can also be seen in co-cultures with monocytes. (FIG. 2A-D).

NK cells co-cultured with OCs were able to lyse OSCSCs significantly more than NK cells co-cultured with monocytes or DCs, and there was a significant increase from day 9 to day 15, co-relating with the higher expansion of NK cells in co-culture with OCs on day 15 (FIG. 2E). IL-2 and anti-CD16 mAb activated NK cells cultured with OCs secreted significantly higher amounts of IFN-γ, compared to NK cells co-cultured with monocytes or DCs (FIG. 2F). Upon analysis of NK cell expansion rate and population doubling (defined by the log of the ratio of the final count to the baseline count divided by the log of 2) it was found that OCs expanded NK cells 21,000-132,000 fold at day 20 and 300,000-5,100,000 fold on day 31, with 17-21 population doublings within 4 weeks (FIG. 2G-H).

Freshly isolated monocytes were compared with mature DCs and OCs for expression of key surface receptors. CD54 was upregulated on DCs and OCs, whereas MHC-I was decreased on DCs and OCs, when compared to monocytes (FIG. 1D). Killer cell immunoglobulin-like receptors (KIR), KLRG1, and MICA/B were higher on OCs, intermediate on monocytes, and very low on DCs (FIG. 2I). ULBP1-6 was high on monocytes, intermediate on OCs and low on DCs (FIG. 2I). NK cell receptors including CD94 and NKG2D were higher on OC-expanded NK cells (FIG. 2J lower row) as compared to untreated primary NK cells (FIG. 2J upper row). KIR2 and KIR3 expression were intermediate on expanded NK cells (FIG. 2J lower row).

Residual Population of T Cells Purified from OC-Expanded NK Cells do not Mediate Cytotoxicity but Secrete IFN-γ

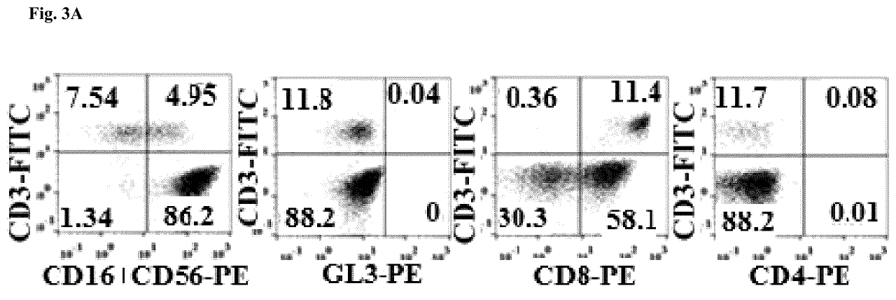

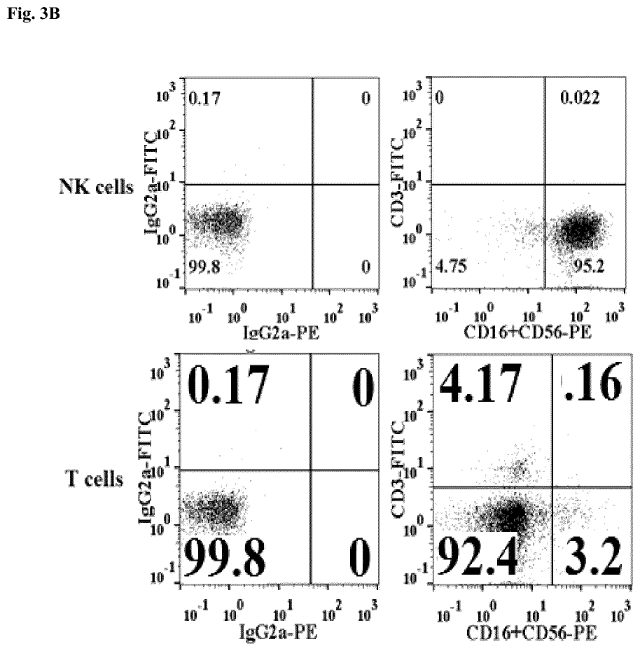

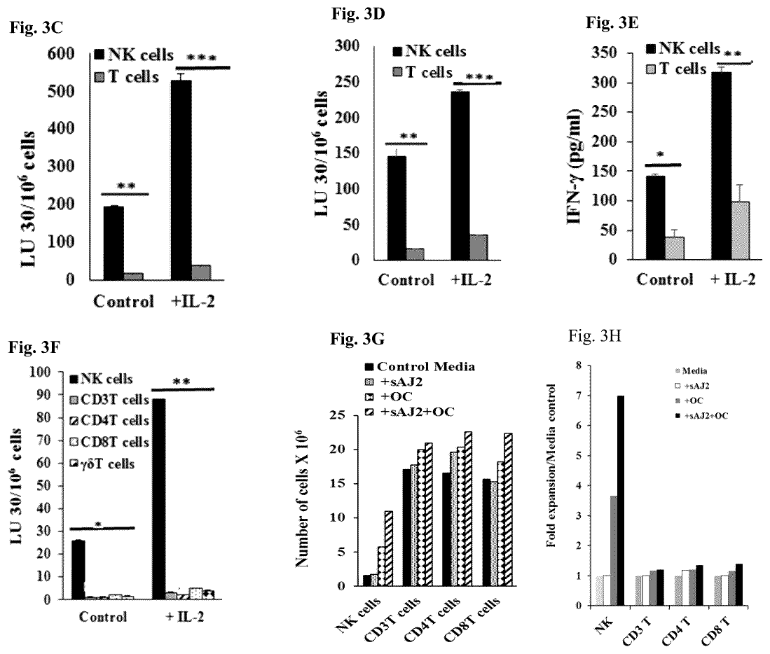

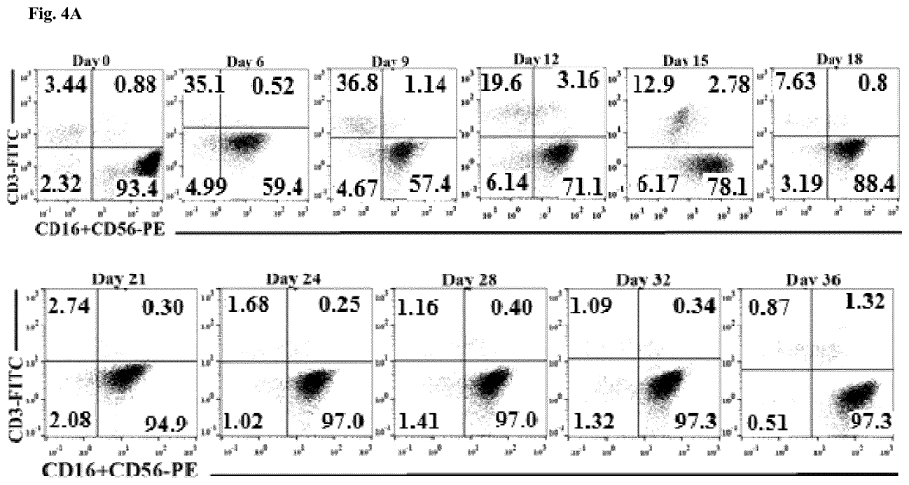

The majority of T cell contaminants from OC-expanded NK cells were CD8+ T cells (FIG. 3A). T cell contaminants from day 9 OC-expanded NK cells were sorted out to obtain purified T cells and NK cells. NK cells were then tested for purity using CD16 and CD3/56 antibodies (FIG. 3B). NK cells and T cells were then treated with IL-2 for 18-20 hours before they were used in ⁵¹Cr release assay against OSCSCs and K562s. CD3+ T cells isolated from OC-expanded NK cells failed to lyse OSCSCs (FIG. 3C) or K562s (FIG. 3D). Supernatants from NK cells secreted significantly higher levels of IFN-γ compared to T cells (FIG. 3E).

Expansion of NK Cells with Osteoclasts Remained High in the First Month, Gradually Reduced in the Second Month and Decreased Substantially in the Third Month

NK cells cultured with OCs expanded for 31-36 days while the rate of contaminating T cells remained low (FIG. 4A). Day 36 expanded NK cells were re-cultured with OCs for a second round of expansion and the NK expansion was continued for 27 days (FIG. 4B). Similarly, the rate of T cell expansion remained very low in the second round of NK cell expansion with OCs (FIG. 4B). Day 67 expanded NK cells were re-cultured with OCs for the third round of expansion, however, NK cells were gradually lost due to T cell expansion (FIG. 4C). No or slight levels of cell death could be observed in the expanding NK cells in the 3 rounds of expansion with OCs (FIG. 4M). The ability of NK cells to lyse cancer stem cells and secrete IFN-γ was gradually decreased from the first to second round of expansion, and in the third round, during which greater percentages of T cells were expanding, these functions became minimal (FIG. 4N-S).

### Osteoclasts, but not K562 or OSCSCs, Expand NK Cells and Increase NK Cell Function.

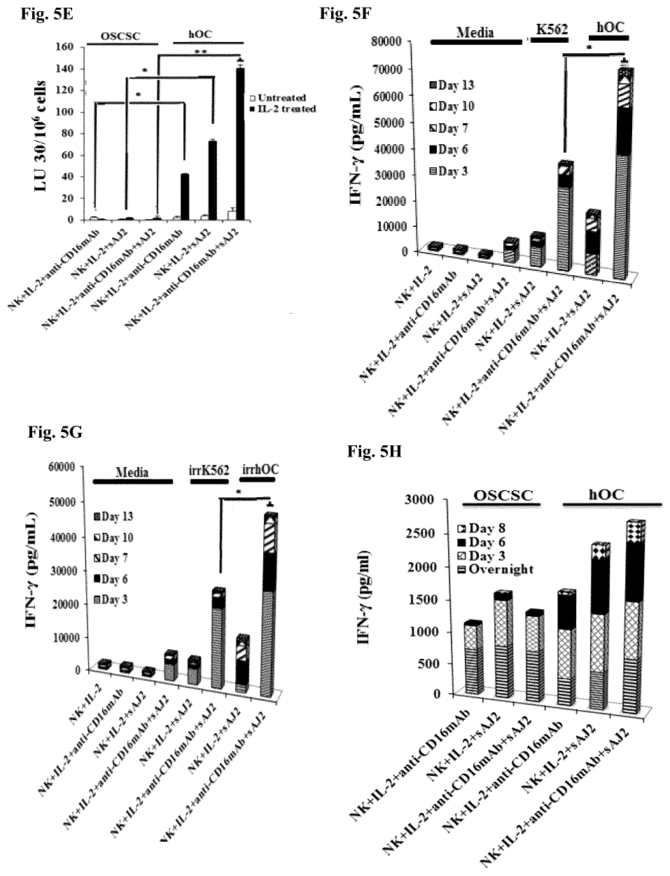

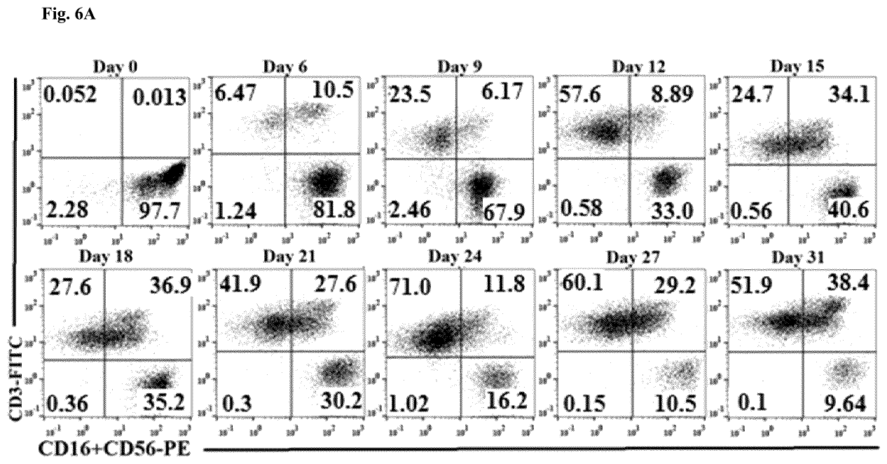

Activated NK cells were cultured with OSCSC, K562, OC, irradiated K562, or irradiated OC in the presence of sAJ2 and the levels of NK expansion and their function were determined (FIG. 5A-5H). NK cell expansion and function (cytotoxicity and IFN-g secretion) induced by either non-irradiated, irradiated K562 or OSCSCs was significantly lower than those induced by non-irradiated or irradiated OCs (FIG. 5).

Decreased Cytotoxicity and Lower IFN-γ Secretion by NK Cells from Patients Coincides with Increased Expansion of T Cells

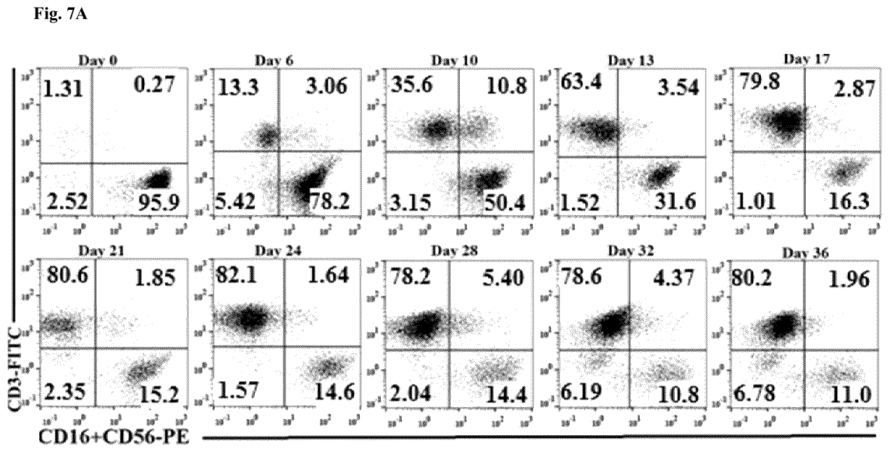

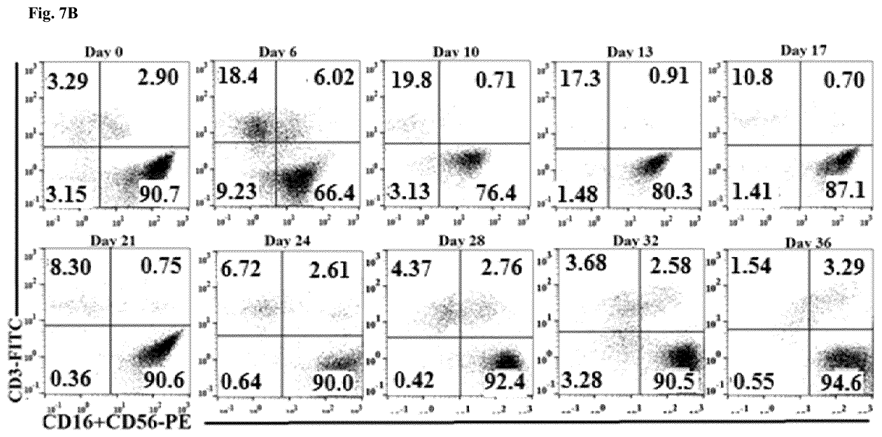

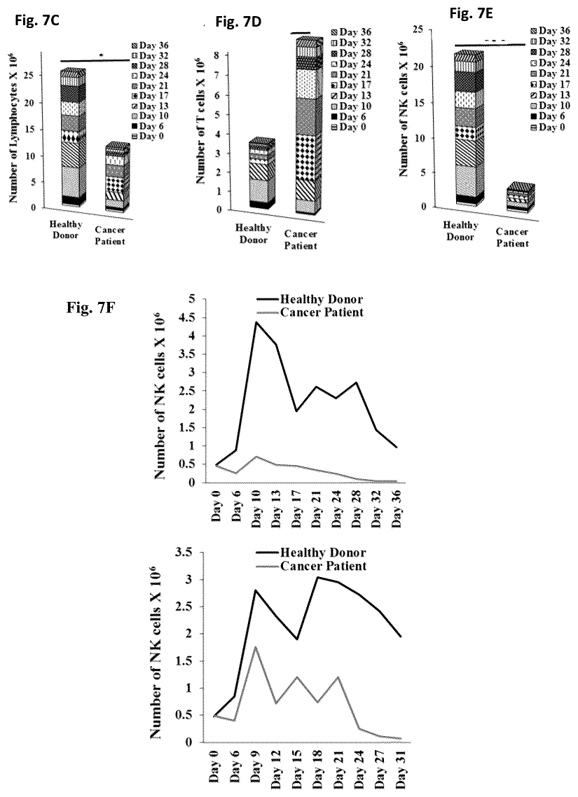

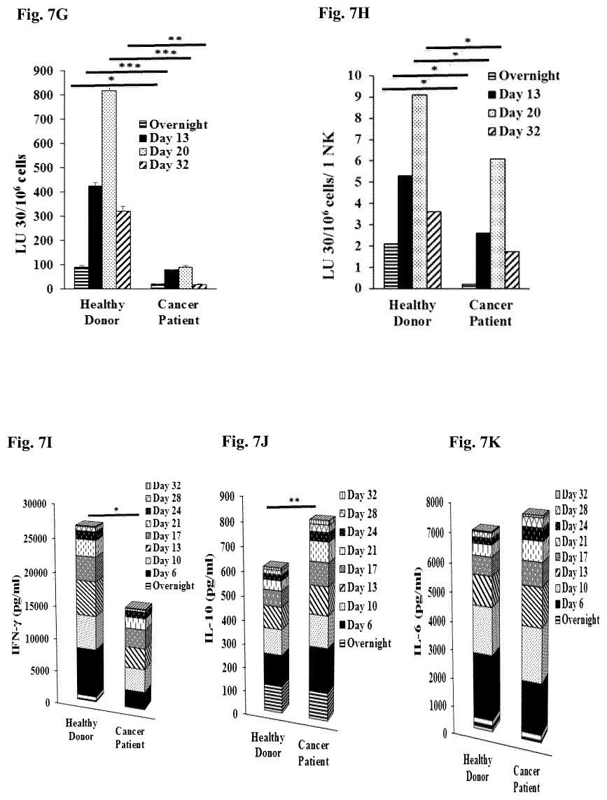

When cultured with OCs, purified NK cells from cancer patients were unable to maintain the expansion of NK cells and indeed, by day 12, greater than half of the expanding cells were T cells. Moreover, by day 31, only 10% NK cells were left in the culture (FIG. 6A and FIG. 7A). In addition, when total numbers of expanded NK and T cells were determined within 31-36 days of expansion in cancer patients, there were less expanding cells from cancer patients when compared to healthy controls (FIG. 6D and FIG. 7C), and the levels of expanding T cells were significantly higher than NK cells (FIGS. 6E-F and FIG. 7D-F). In contrast, NK cells isolated from healthy donors maintained the expansion of NK cells and the levels of NK expansion were significantly higher than T cells (FIG. 6B, E, F and FIG. 7B, 7E, 7F). No or much lower proliferation of NK cells were observed with patient NK cells when compared to healthy NK cells at different days of culture (FIG. 7F). No significant cell death could be observed in the expanding cells either from healthy donor or patient, although the death rate was slightly higher in cells from the patient than healthy donor (FIG. 6C).

Patient's NK cells cultured with OCs lysed OSCSCs significantly less when compared with the healthy NK cells cultured with OCs (FIG. 6G and FIG. 7G). When normalized based on the number of NK cells, cytotoxicity induced per NK cell by patients was less when compared to NK cells from healthy controls (FIG. 6H and FIG. 7H). OC-expanded patient NK cells secreted significantly less IFN-γ when compared to healthy OC-expanded NK cells (FIG. 6I and FIG. 7I). OC expanded oral cancer patients' NK cells secreted significantly less IL-10 when compared to healthy NK cells (FIG. 6J), whereas those from pancreatic cancer patients secreted higher IL-10 when compared to healthy NK cells (FIG. 7J). No significant differences could be observed for the levels of IL-6 secretion by healthy or cancer patients NK cells (FIG. 6K and FIG. 7K). The levels of NKG2D surface expression were similar whereas CD94 expression was higher and KIR2, NKp30, NKp44 and NKp46 expressions were lower on the surface of patients NK cells as compared to healthy control NK cells expanded by the osteoclasts (FIGS. 6L and 6M).

Supernatants from Patient Expanded NK Cells have Much Lower Ability to Differentiate OSCSCs

OSCSCs were treated with equal volumes of day 13 supernatants from patient and healthy donors for 18-20 hours before the levels of CD44, B7H1, CD54, and MHC-I expressions were analyzed (FIG. 6N). There was a 7.1 fold increase in MHC-I expression by healthy NK supernatants but only 2.56 fold increase induced by patient NK supernatants (FIG. 6N). For CD54 expression, a 13-fold increase was observed by healthy NK supernatants compared to a 2.1 fold increase with patient NK supernatants. As for B7H1, a 3.75 fold increase was observed by healthy NK supernatant compared to a 1.5 fold increase with patient NK supernatants. CD44 was decreased by healthy NK supernatants, whereas no decrease was observed by patient NK supernatants (FIG. 6N). No significant cell death could be observed after treating OSCSCs with NK supernatants (FIG. 6O). As shown in FIG. 6P, 74% decrease in NK cell-mediated cytotoxicity was observed when treated with healthy NK supernatants, whereas only 33% decrease could be observed with patient NK supernatants (FIG. 6P).

Oral Tumors in Humanized Mice Preferentially Expand T Cells Resulting in the Loss of NK Cytotoxicity while Retaining IFN-γ Secretion.

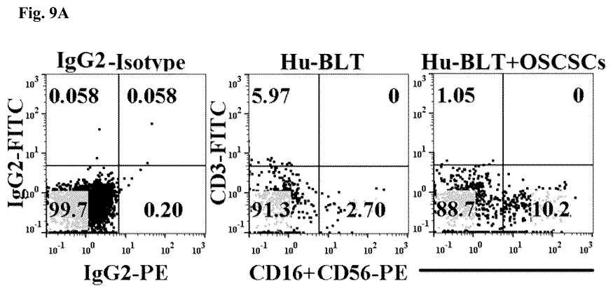

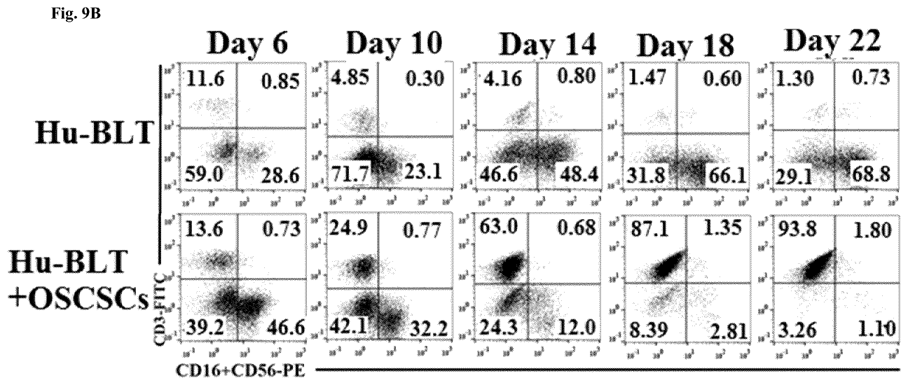

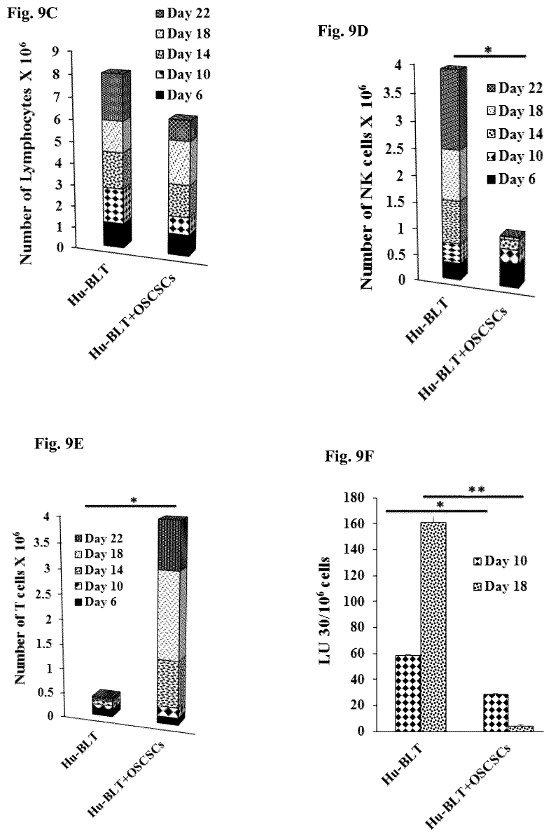

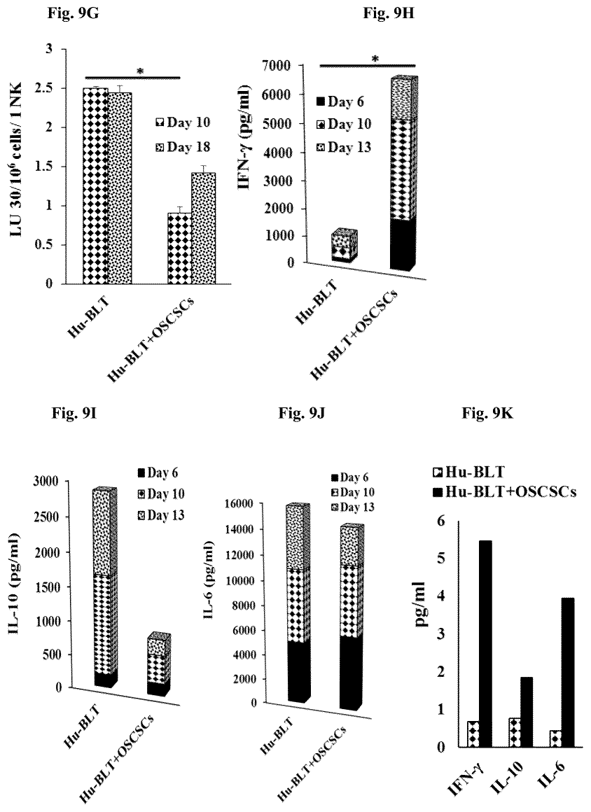

Humanized-BLT mice were implanted with oral tumors and mice were sacrificed 4 weeks after tumor implantation. The spleens from hu-BLT mice were harvested, and T cells were sorted out. The flow-through cells containing B cells (FIG. 8) were then treated with IL-2 and anti-CD16mAb for 18-20 hours before they were cultured with BLT-OCs. Even though tumor bearing hu-BLT mice contained larger percentages of NK cells (FIG. 9A), the expansion resulted in gradual and significant T cell expansion which started on day 6 and continued to day 22, at which point 96% of the cells were T cells and only 1.1% were NK cells. In contrast, flow-through cells from hu-BLT mice with no tumor which contained less NK cells initially, expanded NK cells, and the levels rose from 28.6% NK cells at day 6 to 69% NK cells at day 22 (FIG. 9B). The levels of NK cells when cultured with autologous OCs were increased in both animals from the initial day of culture, although no tumor mice had a 10.59 fold increase from day 0 to day 6, whereas the mice with tumor had a 4.56 fold increase (FIG. 9B). The total numbers of expanded lymphocytes were low in tumor-bearing mice as compared to those without tumors (FIG. 9C), with the majority being T cells and not NK cells (FIGS. 9D and E).

NK enriched cells from tumor-bearing mice, when co-cultured with OCs, were able to lyse OSCSCs significantly less than those of control mice without tumor (FIG. 9F). In addition, when cytotoxicity was assessed per NK cell basis, less cytotoxicity was seen with NK cells from tumor-bearing mice as compared to NK cells from control mice without tumor (FIG. 9G). NK enriched cells from tumor-bearing mice secreted significantly higher IFN-γ (FIG. 9H), lower IL-10 (FIG. 9I), and slightly lower IL-6 (FIG. 9J) when compared to the control mice without tumor. Sera from peripheral blood of tumor bearing hu-BLT mice exhibited increased secretion of IFN-γ, IL-10, and IL-6 as compared to the control mice with no tumor (FIG. 9K).

### IL-15, in Part, Mediates Expansion of NK Cells by Osteoclasts, Whereas IL-12 is Responsible for Increased IFN-γ Secretion by NK Cells

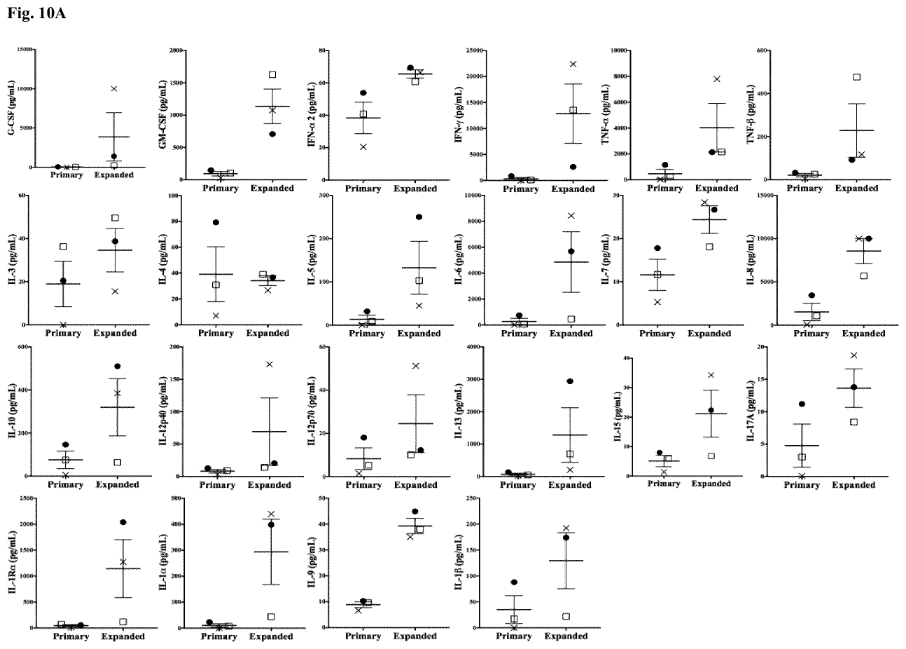

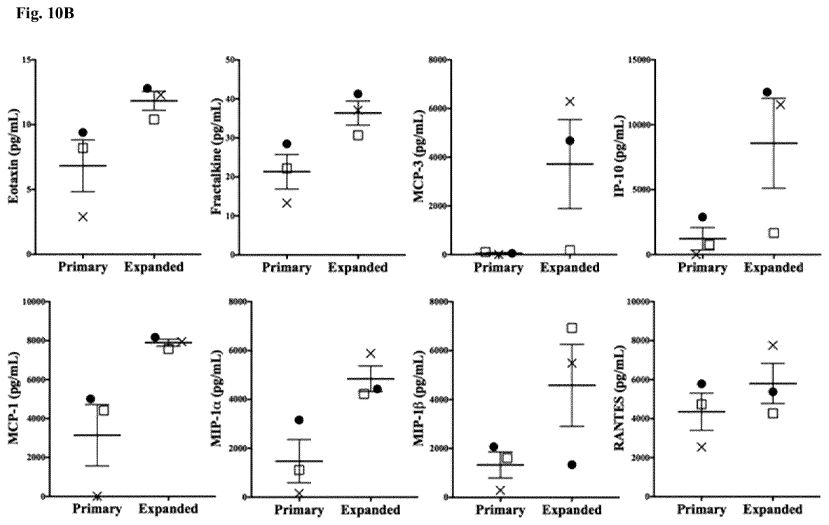

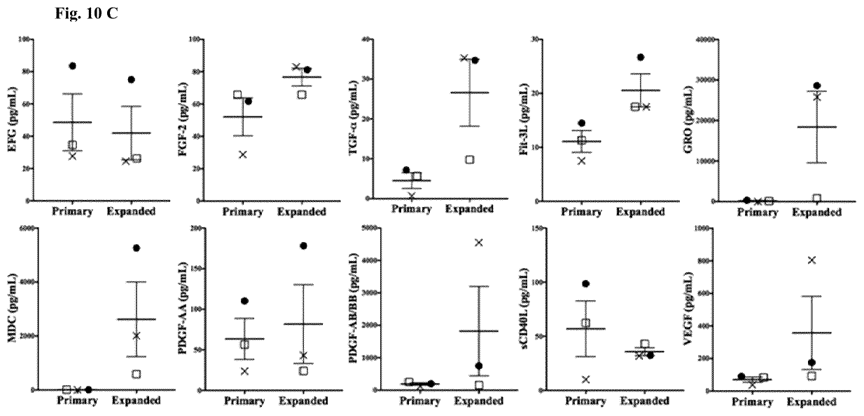

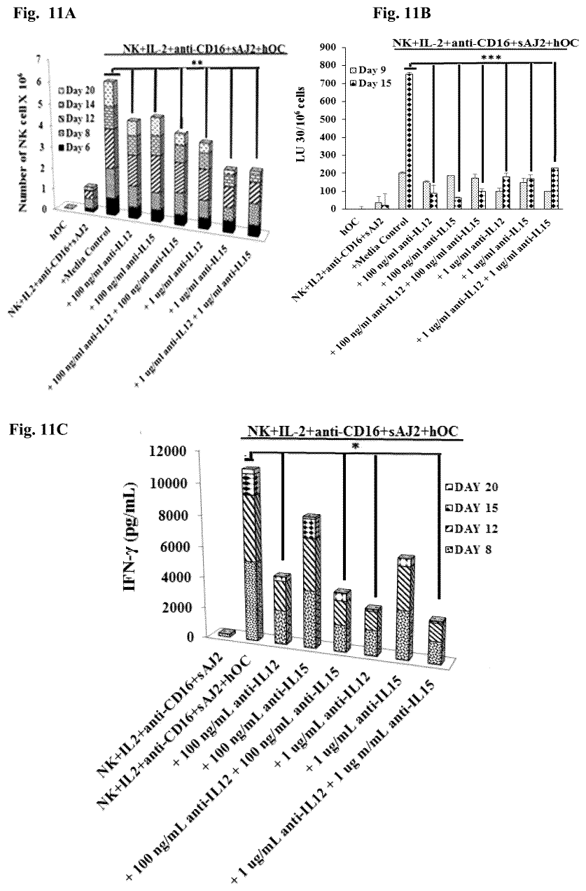

We determined the levels of cytokines, chemokines, growth factors, and ligands secreted by primary NK cells and day 6 OC-expanded NK cells (FIG. 10). The majority of secreted cytokines, chemokines, growth factors, and ligands were higher by OC-expanded NK cells when compared to those secreted by the primary NK cells activated with IL-2 and anti-CD16 mAb (FIG. 10). 50-60 fold higher induction of IL-12 and 20-26 fold higher IL-15 secretion were seen by OC-expanded NK cells as compared to primary NK cells (FIG. 10). Addition of anti-IL-12 and/or anti-IL-15 mAbs significantly reduced cell expansion with 1 μg/ml of anti-IL-15 having the largest effect (FIG. 11A). The treatment with anti-IL-12 and/or anti-IL-15 did not affect the cytotoxic function of NK cells at day 9 (FIG. 11B), but was inhibited significantly at day 15 (FIG. 11B). The levels of IFN-γ secretion by OC-expanded NK cells were reduced more by anti-IL-12 when compared to anti-IL-15 treatment (FIG. 11C).

### Addition of Anti-CD3 Antibody Controls T Cell Expansion and Increases OC-Expanded NK Cells

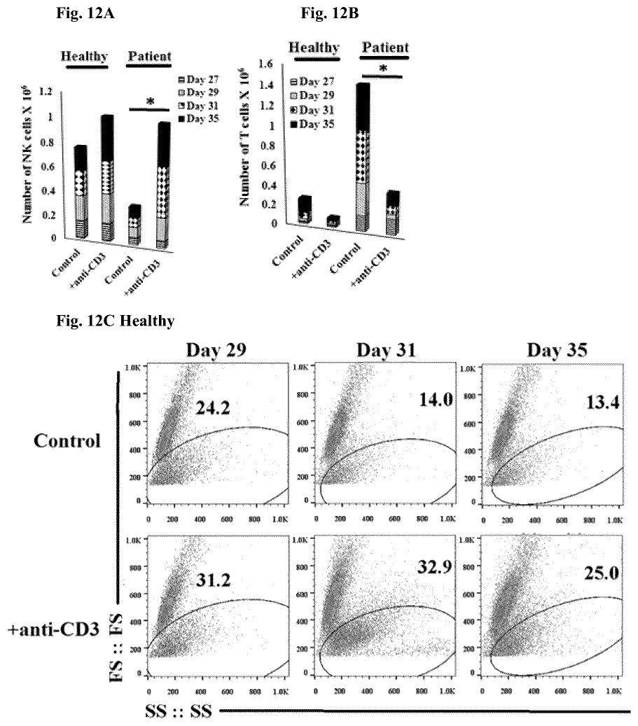

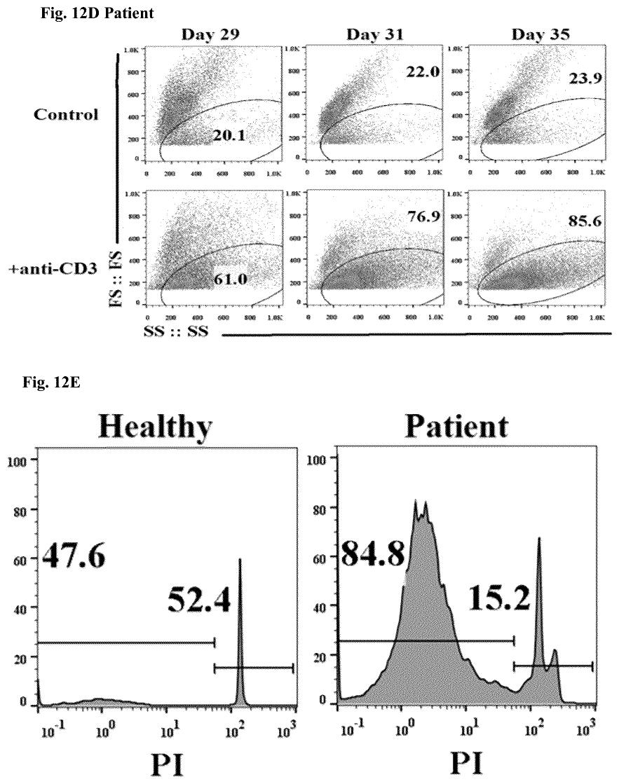

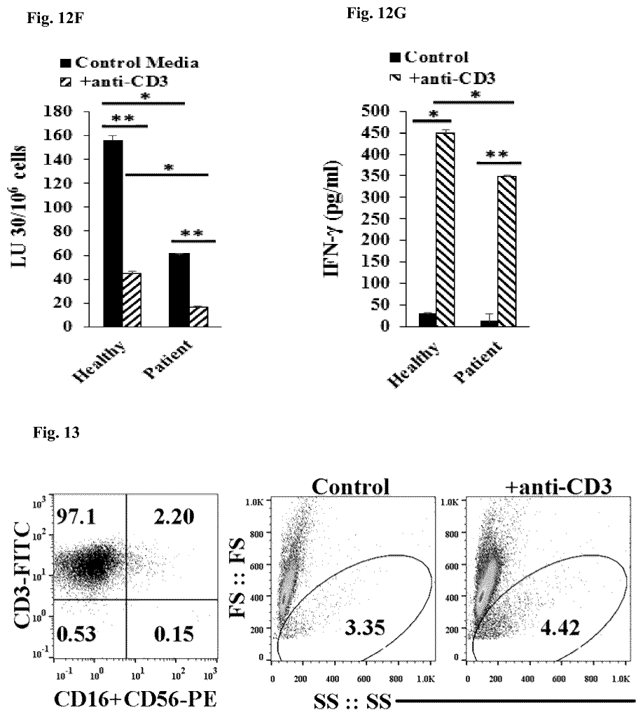

Lymphocytes were treated with anti-CD3 and NK or T cell expansions were assessed in different days (FIGS. 12A and 12B). The levels of cells which had lost both forward and side scatter increased (FIGS. 12C and 12D), and elevation in DNA fragmentation as evidenced by an increase in sub G0/G1 peak in cell cycle analysis was obtained in anti-CD3 treated cells indicating the loss of T cells (FIG. 12E). Accordingly, the levels of NK cells rose in both patient and healthy donors (FIG. 12A-12B). The population which lost forward and side scatter was CD3+ T cells as determined by CD3 and CD16 mAb staining (data not shown). In the absence of NK cells anti-CD3 mAb treated T cells did not lose forward and side scatter (FIG. 13) nor exhibited cell death (data not shown). When cytotoxicity of NK cells was assessed before and after the addition of anti-CD3 mAb, NK cells from both healthy and patient donors had significantly lower cytotoxicity, although the levels still were higher with healthy NK cells (FIG. 12G). In contrast the levels of IFN-γ secretion rose significantly in both healthy and patient NK samples indicating the induction of split anergy in NK cells by anti-CD3mAb bound T cells (FIG. 12F).

### Osteoclast-Activated NK Cells Substantially Increase CD8+ T Cell Numbers

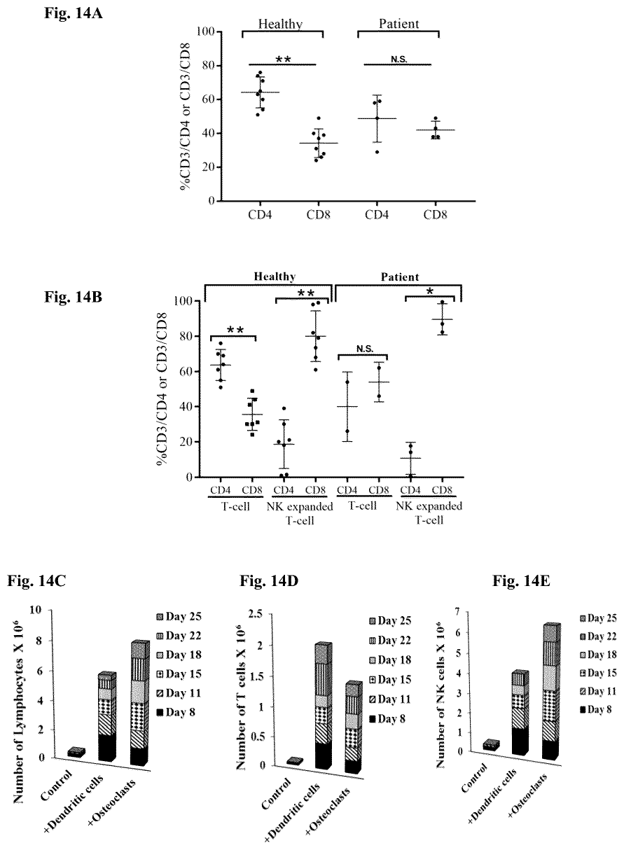

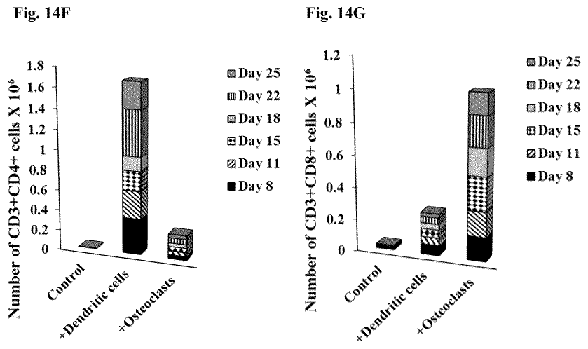

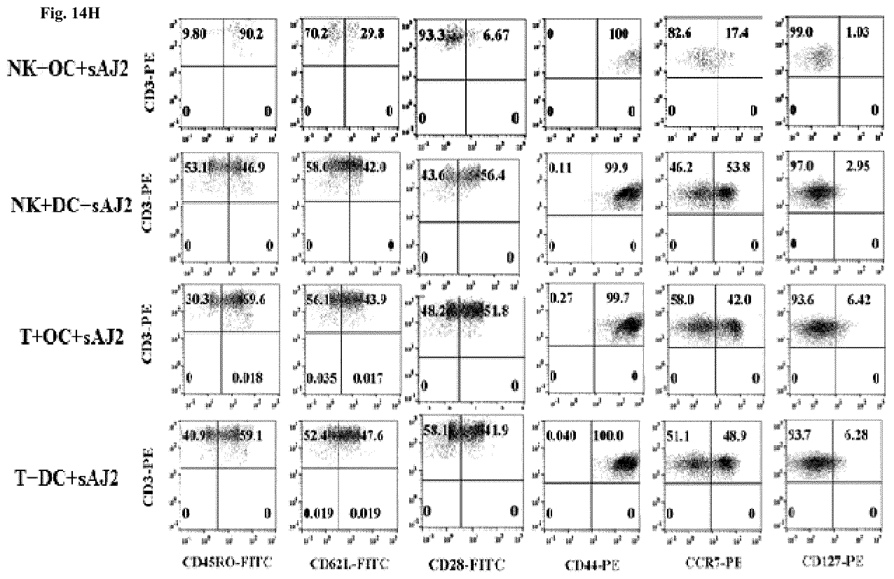

When compared with healthy individuals, cancer patients have on average moderately higher percentages of CD8+ T cells when compared to healthy controls, and lower percentages of CD4+ T cells (FIG. 14A). When cultured with osteoclasts T cells in the absence of NK cells failed to expand CD8+ T cells, however, purified NK cells activated with OCs which contained a very small fraction of contaminating T cells, expanded CD8+ T cells from both healthy and patient cultures, albeit patient NK cell cultures expanded T cells faster than healthy NK cells (FIG. 14B). When OC activated NK cell expanded T cells were compared to DC activated NK cell expanded T cells, OCs preferentially expanded NK cell mediated CD8+ T cells whereas DCs expanded NK cell mediated CD4+ T cells (FIG. 14C-FIG. 14G). In addition, OC activated NK cell expanded CD8+ T cells were all of activated phenotype since they were CD45RO+CD44+CD62L−/lowCCR7−/low indicating memory/effector phenotype (FIG. 14H).

In vivo and in vitro studies recently showed that osteoclasts are major activators of NK cells. More importantly, single monthly stimulation with osteoclasts were able to maintain expansion of super-charged NK cells for over two months from healthy donors. In addition, the cytotoxic function of NK cells remained significantly high in the first month and declined in the second month of expansion. It is unclear why NK cell function continued to decline at the second month of stimulation even though large numbers of NK cells continue to expand. It is possible that additional signals are required for the maintenance of NK cell cytotoxicity at the second month and/or more frequent supplementation with OCs is required.

Using OCs as feeder cells was the best strategy to expand large numbers of NK cells compared to those previously described (Table 1). First, Applicants obtained 21,000-132,000 fold expansion on day 20, and 0.3-5.1 million on day 31, with 17-21 population doublings within 4 weeks of expansion, which is a much higher rate than any previously reported method (Table 1). Although cytotoxic function of expanded NK cells across studies is difficult to compare due to different types of targets used, the strategy disclosed herein provides large numbers of NK cells with significant capabilities to target and lyse cancer stem cells and secrete IFN-γ (Table 1). Moreover, primary NK cells were expanded with no or very little chance of transformation, which is known to influence NK cell killing potential since most NK lines (such as YT, NK92 or NK-L) or transfected NK cells tend to either lose most or some of their cytotoxic and IFN-γ secretion capabilities respectively. In addition, although anti-CD3 antibody treatment to maintain NK cell expansion would be a great strategy to limit T cell expansion, the cytotoxic activity of such NK cells is lower due to significant split anergy induced in NK cells. This strategy could be great for inducing differentiation of the tumors due to a larger increase in IFN-γ secretion; however, it is much less optimal for the elimination of the tumors. This could be one reason why the use of NK cells in immunotherapy of solid tumors did not result in regression of the tumors in a small numbers of patients tested.

TABLE 1

| NK | Method of | Feeder | | | |
|---|---|---|---|---|---|
| Source | selection | cells | Stimulation | Expansion | References |
| PBMC | Negative | Autologous | IL-2, anti- | 21,000-132,000 | Current |
| | selection | and | CD16 | fold expansion | Specification |
| | for NK | allogeneic | mAb, | at 20 days, | |
| | | osteoclasts | sAJ2 | 0.3-5.1 | |
| | | | | million at 31 | |
| | | | | days; 17-21 | |
| | | | | population | |
| | | | | doublings | |
| | | | | with an | |
| | | | | average of | |
| | | | | 19 population | |
| | | | | doublings | |
| | | | | within 4 weeks | |
| PBMC | Whole | None | IL-2, anti- | 637-5712 | Deng et al. |
| | PBMCs | | CD16Ab- | fold at | (2012) |
| | | | immobilized | 21 days | International |
| | | | flask, | | immuno- |
| | | | OK432 | | pharmacology |
| | | | | | 14(4):593-605 |
| PBMC | Negative | Irradiated | IL-2 and | 100 fold | Luhm et al., |
| (negative | selection | autologous | IL-15, | at 16 days | (2002) Journal |
| selection) | for NK | PBMC's | (phytohe- | | of hemato- |
| | | (pretreated | magglutinin | | therapy & |
| | | for 3-5 | (PHA) and | | stem cell |
| | | days with | ionomycin | | research 11(4): |
| | | IL2, IL-15, | first 24 | | 651-657 |
| | | and con- | hours) | | |
| | | canavalin | | | |
| | | A) | | | |
| PBMC | CD3 | Autologous | IL-2 | 79-322 | Parkhurst et |
| | depletion | irradiated | | fold at | al., (2011) |
| | of PBMCs | PBMCs | | 21-26 days | Clinical |
| | | stimulated | | | cancer |
| | | with OKT3 | | | research: an |
| | | | | | official journal |
| | | | | | of the American |
| | | | | | Association for |
| | | | | | Cancer |
| | | | | | Research 17 |
| | | | | | (19):6287-6297 |
| PBMC | Whole | Wilms tumor | IL-2 | 58-401- | Harada et al.. |
| | PBMCs | cell line | | fold at | (2002) Japanese |
| | | (HFWT) | | 10-21 days | journal of |
| | | | | | cancer research: |
| | | | | | Gann 93(3): |
| | | | | | 313-319 |
| PBMC | Whole | K562-mb15- | IL-2 | 165 | Voskensetal. |
| | PBMCs | 41BBL | | (4-567) | (2010) Journal |
| | | | | fold at 14 | of experimental |
| | | | | days | & clinical |
| | | | | | cancer research: |
| | | | | | CR 29:134 |
| PBMC | Whole | K562-mb15- | IL-2 | 21.6 | Fujisaki et al. |
| | PBMCs | 41BBL | | (5.1-86.6) | (2009) Cancer |
| | | | | fold at | research 69(9): |
| | | | | 7 days | 4010-4017 |
| PBMC | Whole | K562-GM | IL-2 | 500 fold | Yang et al. |
| | PBMCs | (transmembrane | | at 21 days | (2015) Cell |
| | | expression of | | | biochemistry |
| | | IL-15, 4- | | | and biophysics |
| | | 1BBL, and | | | 73(3):723-729 |
| | | IL-18) | | | |
| PBMC | CD3 | Irradiated | IL-2 | 490 | Berg et al. |
| | depletion | EBV-TM- | | (+/−260) | (2009) |
| | followed | LCL | | at 21 days | Cytotherapy |
| | by | | | | 11(3): 341-355 |
| | CD56+ | | | | |
| | selection | | | | |
| Cord | CD34+ | None | SCF, TPO, | >15,000 | Spanholtz |
| Blood | positive | | 1L-7, Fit3L, | fold at 5 | (2010) PloS |
| (CB) | selection | | IL-15, IL-2, | weeks | one 5(2): |
| | | | G-CSF, | | e9221 |
| | | | GM-CSF, | | |
| | | | IL-6, LIF, | | |
| | | | MIP-1α | | |
| Cord | CD34+ | None | SCF, Fit3L, TPO, | >2,000 | Spanholtz |
| Blood | positive | | IL-7, GM-CSF, | fold at 6 | (2011) PloS |
| (CB) | selection | | G-CSF, IL-6, | weeks | one 6(6): |
| | | | IL-15, LMWH, | (using | e20740 |
| | | | and IL-2 | GMP) | |
| NK-92 | N/A | None | IL-2 | 218-250 | Tam et al., |
| | (cell line) | | | fold at | (2003) Cyto- |
| | | | | 15-17 days | therapy 5(3): |
| | | | | | 259-272 |
| PBMC | Whole PBMCs | K562-mb15- | IL-2 | TERT | Fujisaki et |
| (genetically | used initially, | 41BBL | | transformed | al. (2009) |
| modified | residual | (continued | | (130-227 | British |
| with | T-cells | stimulation) | | population | journal of |
| TERT for | removed | | | doublings | haematology |
| immortal- | after 7 days | | | over 1000 | 145(5): |
| ization) | using | | | days) Non- | 606-613 |
| | anti-CD3 | | | transformed | |
| | Dynabeads | | | (11-20 | |
| | | | | population | |
| | | | | doublings at | |
| | | | | 8-15 weeks) | |
| PBMCs | Whole | K562-mb15- | IL-2 | N/A | Chang et al., |
| (after 7 days | PBMCs used | 41BBL | | | (2013) |
| of culture, | initially, | | | | Cancer |
| retrovirally | residual T- | | | | research |
| transduced | cells removed | | | | 73(6): |
| with | after 7 days | | | | 1777-1786 |
| NKG2D- | using anti- | | | | |
| DAP10- | CD3 | | | | |
| CD3ζ) | Dynabeads | | | | |

In contrast to OCs, DCs stimulated preferential expansion of small numbers of contaminating T cells in purified NK cells, and the levels of T cells continued to rise for a month thereby decreasing the NK expansion. Accordingly, lower levels of cytotoxicity against OSCSCs were seen. Interestingly, when the cytotoxicity of NK cells was assessed at an earlier time point in which similar proportion of NK cells was observed with OC and DC cultures, a rise in NK cytotoxicity could be observed by the NK cells cultured with OCs (data not shown). Similarly, there was an increase in IFN-γ secretion by NK cells cultured with OCs when compared to those cultured with DCs indicating that on a per cell basis, NK cells secreted higher IFN-γ when cultured with OCs as compared to those cultured with DCs (FIG. 1C). T cells sorted out from OC-expanded NK cells had very low cytotoxicity and the minimal cytotoxicity seen by the T cells was likely due to contaminating NK cells (FIG. 3C and FIG. 3B). On the other hand, T cells likely released IFN-γ, but the levels were significantly lower than those released by NK cells (FIG. 3E). When subsets of T cells; CD8, CD4 and gdT cells were sorted out and used in cytotoxicity assay, only NK cells were able to lyse these cells and the residual cytotoxicity seen by the T cell subsets was due to contaminating NK cells (FIG. 3F). OCs in comparison to DCs displayed higher expression of activating NK ligands, whereas they both demonstrated similar levels of differentiation antigen CD54. Interestingly, they both demonstrated lower levels of MHC class I expression when compared to monocytes (FIG. 1D). In addition, IL-15 secretion appeared to be important for the proliferation of NK cells (FIG. 11A) whereas IL-12 was important for the secretion of IFN-γ (FIG. 11C). Both cytokines were important in cytotoxic function of NK cells when assessed at the later day as compared to earlier time point in expansion (FIG. 11B).

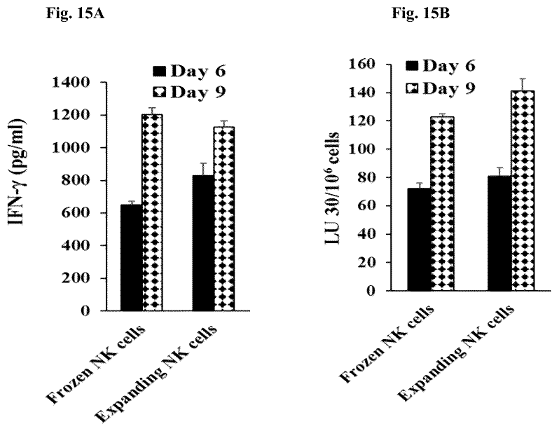

In comparison to OCs, all the tumor cell lines tested regardless of whether they were non-irradiated or irradiated did not support the expansion of NK cells in long term, and in short term even though secretion of IFN-γ could be observed in cultures with K562 and OSCSCs, this effect was short lived (FIG. 5). In addition, OC-induced expansion was also compared to irradiated PBMCs, and it was found to be significantly inferior to that of OCs (data not shown). NK cells expanded by OCs demonstrated much higher levels of activating receptor expression including NKG2D, NKp46, NKp44, NKp30, CD94 and increased inhibitory receptors KIR2 and KIR3 with much lower expression of CD16 receptor when compared to primary NK cells (FIG. 2J, lower row). When comparing OC-expanded NK cells of healthy donors, cancer patients had in general much lower receptor expression, and the levels were even less than those seen on third round of stimulated healthy NK cells which had lost significant cytotoxic and cytokine secretion capabilities, and allowed for the expansion of T cells. Similar to patient NK cells, NK cells expanded by K562 and OSCSCs were short lived and had much lower cytotoxic and cytokine secretion capability. K562 or OSCSCs, unlike OCs, expressed lower levels of NK activating ligands (FIG. 1D), and lacked secretion of key cytokines responsible for the expansion of NK cells, since increase in these signals by OCs were able to elevate expansion and function of NK cells (FIG. 10A). It remains to be determined if such signals by engineered K562s (Table 1) are inferior to those delivered by the OCs since the rate of expansion of NK cells by engineered K562 cells is lower by a magnitude of 100 fold from OC expanded NK cells (Table 1 and FIG. 5A and FIG. 5B). In addition, continuous stimulation by the engineered K562s is required to maintain NK expansion whereas only one stimulation with OCs was enough to expand super-charged NK cells for over a month. Moreover, OC expanded NK cells, unlike primary NK cells, withstand freezing temperatures quite well and retain their super-charged characteristics and expansion rates with no loss of viability or function (FIG. 15).

When autologous or allogeneic OCs was used to expand NK cells from cancer patients a very distinct profile was observed. Cancer patients' OCs also expanded T cells early in the culture with decreased overall expansion of NK cells at different days. When assessing the function of patient NK cells after OC cultures a significant loss of NK cell cytotoxicity, and decrease in IFN-γ secretion could be observed per NK cells (FIG. 6H and FIG. 15G). This observation is important since it indicated that faster expansion of contaminating small fraction of T cells in purified NK cultures in cancer patients correlates with loss of cytotoxic function of NK cells. Thus, the loss of NK cells may also provide the fertile ground for the growth and metastasis of cancer stem cells.

To test whether OCs obtained from humanized mice implanted with tumors, similar to cancer patients OCs expand contaminating small fraction of T cells within purified NK cultures faster than their non-tumor bearing counter parts, we implanted and grew OSCSCs in the floor of the mouth. After five weeks of growth the mice were euthanized and T cells were depleted before the cells were cultured with autologous and allogeneic OCs and the rate of NK cell expansion were determined. Similar to healthy donor NK cells, NK cells from hu-BLT mice without tumor expanded NK cells for a longer period of time, whereas those from tumor bearing mice expanded the small fraction of contaminating T cells within the NK cultures faster favoring the expansion of T cells over NK cells. Interestingly, similar to the loss of NK cell cytotoxicity observed in cancer patients we also observed significant loss of NK cell cytotoxicity in hu-BLT mice implanted with tumors which may be the underlying mechanism for the expansion of T cells. However, OC-expanded NK+ T cells from the tumor bearing mice secreted higher levels of IFN-γ when compared to those without tumors, suggesting the potential induction of split anergy in NK cells to drive differentiation of cancer stem cells. This was found to be the case since single cell preparation of tumors in NK injected tumor bearing hu-BLT mice demonstrated higher differentiation antigens and were resistant to NK cell mediated cytotoxicity.

It is hypothesized that the microenvironment in humanized mice is not conducive of the expansion of NK cells due to a lack of cross reactivity between murine and human IL-15, since the addition of human IL-15 promotes an increase in NK cells. Unlike NK cells, which require signals from osteoclasts for their expansion, T cells expand rapidly in the absence of osteoclasts, and osteoclasts stimulate T cell expansion moderately (FIG. 3G and FIG. 3H). It is likely that signals received from the mouse tissue microenvironment is adequate to maintain T and B cell proliferation whereas NK cell proliferation requires signals from myeloid subset. Interestingly, the frequencies of both NK and myeloid subsets are decreased in peripheral blood and in the tissues whereas in the bone marrow, rich with myeloid cells the levels of T and NK cells remain similar. Whether DCs favor expansion of T cells in the periphery and osteoclasts favor expansion of NK cells in bone marrow requires further studies. Humanized mice are the best and closest model to the human cancer model since in both models lower percentages as well as decreased function of NK cells are found to potentially contribute to cancer progression.

The most exciting finding is the ability of NK cells to increase CD8+ T cells when cultured in the presence of OCs. This data indicates that NK cells are important effectors in the expansion of CD8+ T cells, thereby allowing an increase in the targeting and lysis of tumor cells expressing higher MHC class I. Whether NK cells increase antigen-specific CD8+ T cells thereby increasing the lysis of tumor cells in an antigen specific manner requires future studies. Rapid expansion of T cells and decreased NK cell numbers in peripheral blood/tissues of cancer patients and the humanized mice could be detrimental for targeting MHC class I low targets including cancer stem cells/undifferentiated tumors by NK cells in order to minimize the tumor load. In addition, NK cells also provide large amounts of IFN-γ to promote optimal differentiation of the cancer stem cells, and higher expression of MHC class I for targeting with CD8+ T cells. Thus, restoration of NK cell numbers and function in cancer patients will be important to establish effective tumor control.

### Example 3: Osteoclast Activated Super-Charged NK Cells Preferentially and Rapidly Expand Super-Charged CD8+ T Cells: Increased Dynamics of CD8+ T Cell Expansion by OC-Expanded NK Cells in Cancer Patients and BLT Humanized Mice

Further research was carried out for the study in Example 2 and the results are summarized below.

Residual Population of T Cells Purified from OC-Expanded NK Cells do not Mediate Cytotoxicity but Secrete IFN-γ

The majority of T cell contaminants from OC-expanded NK cells were CD8+ T cells (Supplementary Fig. S2A). T cell contaminants from day 9 OC-expanded NK cells were sorted out to obtain purified T cells and NK cells. NK cells were then tested for purity using CD16 and CD3/56 antibodies (Supplementary Fig. S2B). NK cells and T cells were then treated with IL-2 for 18-20 hours before they were used in ⁵¹Cr release assay against OSCSCs and K562s. CD3+ T cells isolated from OC-expanded NK cells failed to lyse OSCSCs (Supplementary Fig. S2C) or K562s (Supplementary Fig. S2D). Supernatants from NK cells secreted significantly higher levels of IFN-γ compared to T cells (Supplementary Fig. S2E).

Decreased Cytotoxicity and Lower IFN-γ Secretion by NK Cells from Patients Coincides with Increased Expansion of T Cells

When cultured with OCs, purified NK cells from cancer patients were unable to maintain the expansion of NK cells and indeed, by day 12, greater than half of the expanding cells were T cells. Moreover, by day 31, only 10% of the remaining cells in the culture were NK cells (FIG. 3A and Supplementary Fig. S4A). In addition, when total numbers of expanded NK and T cells were determined within 31-36 days of expansion in cancer patients, there were less expanding cells from cancer patients when compared to healthy controls (FIG. 3D and Supplementary Fig. S4C), and the levels of expanding T cells were significantly higher than NK cells (FIG. 3E-FIG. 3F and Supplementary Fig. S4D-E). In contrast, NK cells isolated from healthy donors maintained the expansion of NK cells and the levels of NK expansion were significantly higher than T cells (FIG. 3B, FIG. 3E, FIG. 3F and Supplementary Fig. S4B, S4D, S4E). No or much lower proliferation of NK cells were observed with patient NK cells when compared to healthy NK cells at different days of culture (FIG. 3E and S4D). No significant cell death could be observed in the expanding cells either from healthy donor or patient, although the death rate was slightly higher in cells from the patient than healthy donor (FIG. 3C).

Patient's NK cells cultured with OCs lysed OSCSCs significantly less when compared with the healthy NK cells cultured with OCs (FIG. 3G and Supplementary Fig. S4F). When normalized based on the number of NK cells, cytotoxicity induced per NK cell by patients was less when compared to NK cells from healthy controls (FIG. 3H and Supplementary Fig. S4G). OC-expanded patient NK cells secreted significantly less IFN-γ when compared to healthy OC-expanded NK cells (FIG. 3I and Supplementary Fig. S4H). OC expanded oral cancer patients' NK cells secreted significantly less IL-10 when compared to healthy NK cells (FIG. 3J), whereas those from pancreatic cancer patients secreted higher IL-10 when compared to healthy NK cells (Supplementary Fig. S4I). No significant differences could be observed for the levels of IL-6 secretion by healthy or cancer patients NK cells (FIG. 3K and Supplementary Fig. S4J). The levels of NKG2D surface expression were similar on healthy as compared to patient NK cells expanded by the osteoclasts (FIG. 3L). The intensity of CD94 expression is higher on the surface of patient NK cells as compared to healthy control (FIG. 3L). KIR2, NKp30, NKp44 and NKp46 expressions were lower on the surface of OC-expanded patient NK cells when compared to healthy NK cells (FIG. 3L), whereas KIR3 expression was either the same or lower on the surface of OC-expanded patient NK cells when compared to healthy NK cells (FIG. 3L).

### Osteoclast Activated NK Cells Substantially Increase CD8+ T Cell Numbers

Cancer patients have on average higher percentages of CD8+ T cells when compared to healthy controls, and lower percentages of CD4+ T cells (FIG. 7A). When cultured with osteoclasts T cells in the absence of NK cells failed to expand CD8+ T cells, however, purified NK cells activated with OCs which contained undetectable or a very small fraction of contaminating T cells, expanded CD8+ T cells from both healthy and patient cultures, albeit patient NK cell cultures expanded T cells faster than healthy NK cells (FIG. 7B). T cells isolated from patients had higher levels of CD45RO and lower CD45RA, CD62L, CD28, CCR7 and CD127 when compared to T cells isolated from healthy controls (FIG. 7C)

### OC Activated NK Cells Preferentially Expanded CD8+ T Cells Whereas DC Activated NK Cells Expanded CD4+ T Cells

To determine whether there were differences between the subpopulations of T cells expanded by OC vs. DC activated NK cells, we analyzed the CD4 and CD8 subpopulations in healthy donors. OC activated NK cells preferentially expanded CD8+ T cells whereas DC activated NK cells expanded CD4+ T cells (FIG. 7D-7I). CD8+ T cells expanded by OC activated NK cells exhibited higher CD45RO and CD44, much lower levels of CD62L, CCR7 and CD127 and intermediate levels of CD28 whereas CD4+ T cells expanded by DC activated NK cells exhibited lower levels of CD45RO, intermediate levels of CD44 and higher levels of CD62L, CCR7 with little change in CD127 and lower levels of CD28 (FIG. 7J). T cells activated by either OC or DC in the absence of NK cells exhibited surface profiles similar to those obtained by NK activated DCs with the exception of CD28 expression which resembled that obtained by OC activated NKs (FIG. 7J). The proportions of CD4 and CD8 within CD3+ T cells were similar between PBMCs and those expanded by either OCs or DCs in the absence of NK cells, and no significant levels of PD-1, Tim 3 or KLRG-1 on T cells either activated by OC or DC in the presence or absence of NK cells could be observed (FIG. 7K-FIG. 7M).

### Example 4: Materials and Methods for Example 5

### Cell Lines, Reagents, and Antibodies

RPMI 1640 supplemented with 10% Fetal Bovine Serum (FBS) (Gemini Bio-Products, CA) was used for the cultures of immune cells. OSCSCs and OSCCs were dissociated and grown from the tongue tumors of patients at UCLA, and were cultured with RPMI 1640 supplemented with 10% FBS. Recombinant IL-2 was obtained from NIH-BRB. Flow cytometry antibodies used in this study were obtained from Biolegend (San Diego, CA). Monoclonal anti-TNF-α and monoclonal anti-IFN-g antibodies were either obtained from commercial sources or prepared in our laboratory and 1:100 dilution was found to be the optimal concentration to use for blocking experiments as described previously.

### Purification of Human NK Cells and Monocytes

Written informed consents approved by UCLA Institutional Review Board (IRB) were obtained and all procedures were approved by UCLA-IRB. PBMCs from healthy human donors 380 were isolated, and NK cells and monocytes were purified using isolation kits obtained from Stem Cell Technologies, as described before. The purity of NK cells and monocyte populations was found to be 95% or higher, respectively, based on the flow cytometric analysis.

### Probiotic Bacteria

AJ2 is a combination of 8 different strains of gram-positive probiotic bacteria (Streptococcus thermophiles, Bifidobacterium longum, Bifidobacterium breve, Bifidobacterium infantis, Lactobacillus acidophilus, Lactobacillus plantarum, Lactobacillus casei, and Lactobacillus bulgaricus) used to induce differentiation of stem cells and are selected for their superior ability to induce optimal secretion of both pro-inflammatory and anti-inflammatory cytokines in NK cells. In addition, each strain was grown, and specific colonies were selected after three rounds of subcloning based on the ability to withstand environmental pressures such as temperature and acidity

### Generation of Osteoclasts and Expansion of Super-Charged Human and Hu-BLT-Derived NK Cells

Purified monocytes were cultured in alpha-MEM medium 400 containing M-CSF (25 ng/mL) and RANKL (25 ng/mL) for 21 days, or otherwise specified. Medium was refreshed every 3 days with fresh alpha-MEM containing M-CSF and RANKL. Human purified and hu-BLT enriched NK cells were activated with rh-IL-2 (1000 U/ml) and anti-CD16mAb (3 ug/ml) for 18-20 hours before they were co-cultured with osteoclasts and sonicated AJ2. The culture media was refreshed with rh-IL-2 every three days as described previously. Since ex-vivo expanded NK cells with osteoclasts and sAJ2 had superior cytotoxicity and IFN-g secretion when compared to other expansion methodologies, and survived for a longer period, they were called super-charged NK cells. For sonication, AJ2 was weighed and re-suspended in RPMI 1640 Medium containing 10% FBS at a concentration of 10 mg/ml. The bacteria were thoroughly vortexed, then sonicated on ice for 15 seconds, at 6 to 8 amplitudes. Sonicated samples were then incubated for 30 seconds on ice. After every five pulses, a sample was taken to observe under the microscope until at least 80 percent of cell walls were lysed. It was determined that approximated 20 rounds of sonication/incubation on ice, were conducted to achieve complete sonication. Finally, the sonicated samples (sAJ2) were aliquoted and stored in a −80 degrees Celsius freezer until use.

### Analysis of Human Oral Cancer Cell Growth in Immunodeficient and Humanized Mice

Animal research was performed under the written approval of the UCLA Animal Research Committee (ARC) (2012-101-13A). Humanized-BLT (hu-BLT; human bone marrow/liver/thymus) mice were prepared in our core facility as previously described.

In vivo growth of oral tumors was done by orthotopically implanting tumor cells into 8-10 weeks-old NSG mice or hu-BLT mice in the floor of the mouth. Mice were anesthetized using isoflurane and tumor cells were then transferred by direct injection in the floor of mouth with 10 pl HC Matrigel (Corning, NY, USA). 7 to 10 days after the surgery mice received 1.5·10⁶ super-charged NK cells via tail vein. Mice were fed AJ2 (5 billion bacteria/dose), began feedings one or two weeks before tumor implantation and were fed every 48 hours, throughout the experiment. Mice were euthanized when signs of morbidity were evident. Oral tumors, BM, spleen and peripheral blood were harvested.

Dissociation and Culture of Cells from Tissues of Hu-BLT and NSG Mice

To prepare a single cell suspension of mouse tissues for subsequent analyses, animals were sacrificed BM, spleen, peripheral blood and oral tumor were obtained. The oral tumor was immediately cut into 1 mm³ pieces and placed into a digestion buffer containing 1 mg/ml collagenase II (for fat tissue), 10 U/ml DNAse I, and 1% bovine serum albumin in DMEM and incubated for 20 minutes at 37° C. oven with on a 150-rpm shaker. After digestion, the sample was filtered through a 40 pm cell strainer and centrifuged at 1500 rpm for 10 minutes at 4° C. The pellet was re-suspended in DMEM and cells counted. Single cell suspension from BM and spleen were obtained by digesting tissues, as described previously. PBMCs were obtained using ficoll-Hypaque centrifugation of heparinized blood specimens. The buffy coat containing PBMCs, were harvested, washed and re-suspended in RPMI 1640 medium.

Purification of NK Cells, CD3T-Cells, and Monocytes from Hu-BLT Mice

CD3C T-cells were isolated from hu-BLT splenocytes using T cells selection kit (Stem-Cell Technologies), and the cells depleted of T cells were used as NK-enriched cells; NK cells from hu-BLT mice were isolated using the human CD56c positive selection kit (Stem-Cell Technologies, Canada). Monocytes from hu-BLT mice were isolated from BM cells using human CD14 positive selection kit (eBioscience, San Diego, CA).

### ELISA

Single ELISAs were performed, as described previously. To analyze and obtain the cytokine and chemokine concentration, a standard curve was generated by either two or three-fold dilution of recombinant cytokines provided by the manufacturer.

### Surface Staining and Cell Death Assays

Staining was performed by labeling the cells with antibodies or propidium iodide, as described previously. The cells were washed twice with ice-cold PBS containing 1% BSA. Predetermined optimal concentrations of specific human monoclonal antibodies were added to 1×10⁴ cells in 50 μl of cold-BSA and cells were incubated on ice for 30 min. Thereafter cells were washed in cold PBS-BSA and brought to 500 μl with PBS-BSA. For cell death assay 1×10⁴ cells in 50 μl of cold-BSA were stained with 8 μg/ml propidium iodide and cells were incubated on ice for 10 min, and brought to 500 μl with PBS-BSA. Flow cytometry analysis was performed using Beckman 485 Coulter Epics XL cytometer (Brea, CA) and results were analyzed in FlowJo vX software (Ashland, OR).

51Cr release Cytotoxicity Assay

The ⁵¹Cr release assay was performed, as described previously. Briefly, different numbers of effector cells were incubated with ⁵¹Cr-labeled target cells. After a 4-hour incubation period the supernatants were harvested from each sample and counted for released radioactivity using the gamma counter. The percentage specific cytotoxicity was calculated as follows:

% Cytotoxicity D Experimental cpm−spontaneous cpm Total cpm−spontaneous cpm

LU 30/10⁶ is calculated by using the inverse of the number of effector cells needed to lyse 30% of tumor target cells.

Stem Cell Differentiation with NK Cell Supernatants

NK cells were treated with a combination of anti-CD16 monoclonal antibody (3 μg/mL) and IL-2 (1,000 U/mL) for 18 hours before supernatants were removed and used for differentiation experiments. The amounts of IFN-γ produced by activated NK cells were measured with IFN-γ ELISA (Biolegend, CA, USA). OSCSCs were differentiated with gradual daily addition of increasing amounts of NK cell supernatants. On average, to induce differentiation, a total of 3,500 μg of IFN-γ containing supernatants were added for 4 days to induce differentiation and resistance of OSCSCSs to NK cell-mediated cytotoxicity. Afterwards, target cells were washed with 1×PBS, detached and used for experiments as described previously.

### Statistical Analysis

An unpaired, two-tailed student t-test was performed for the statistical analysis. One-way ANOVA with a Bonferroni post test was used to compare different groups. (n) denotes the number of mice used for the experiment. For cytotoxicity and cytokine analysis either duplicate or triplicate samples were used for assessment. The following symbols represent the levels of statistical significance within each analysis, ***(p value <0.001), **(p value 0.001-0.01), *(p value 0.01-0.05).

### Example 5: Super-Charged NK Cells Inhibit Growth and Progression of Stem-Like/Poorly Differentiated Oral Tumors In Vivo in Humanized BLT Mice

### Single Injection of Super-Charged NK Cells Inhibited OSCSCs Tumor Growth, and Significantly Improved Health of the Mice

Hu-BLT mice were generated and human OSCSCs were implanted in the floor of the mouth of NSG and hu-BLT mice (FIG. 62A and FIG. 62B) and weight loss was monitored on a weekly basis (FIG. 62C). Single injection of super-charged NK cells resulted in lower weight loss of mice implanted with OSCSCs (FIG. 62C). Mice implanted with OSCSCs and injected with NK cells did not exhibit morbidity, and were able to intake food; whereas mice with oral tumors in the absence of NK injection became morbid, had complications in ingesting food due to growing tumors (data not shown) and exhibited rapid weight loss (FIG. 62C). Interestingly, tumor-bearing hu-BLT mice with no NK injection had less weight loss when compared to tumor-bearing NSG mice, indicating that reconstituted human immune cells were able to limit tumor growth slightly but not efficiently (FIG. 62C). Therapeutic effect of NK injection in hu-BLT mice was also seen when the tumor sizes were compared after tumor resection. Tumors from tumor-bearing hu-BLT mice without NK injection were much larger than those of NK-injected tumor-bearing hu-BLT mice (FIGS. 62D and 62E). Tumor weights remained substantially less in NK or NK-injected/AJ2 fed mice (FIG. 62F), in comparison to the large tumors, which were formed in tumor-bearing mice that did not receive NK treatment (FIG. 62D-FIG. 62F). In addition, in agreement with the weight loss data tumor-bearing hu-BLT mice with no NK injection had slightly smaller tumors when compared to tumor-bearing NSG mice, indicating that reconstituted human immune cells were able to limit tumor growth slightly but not efficiently (data not shown).

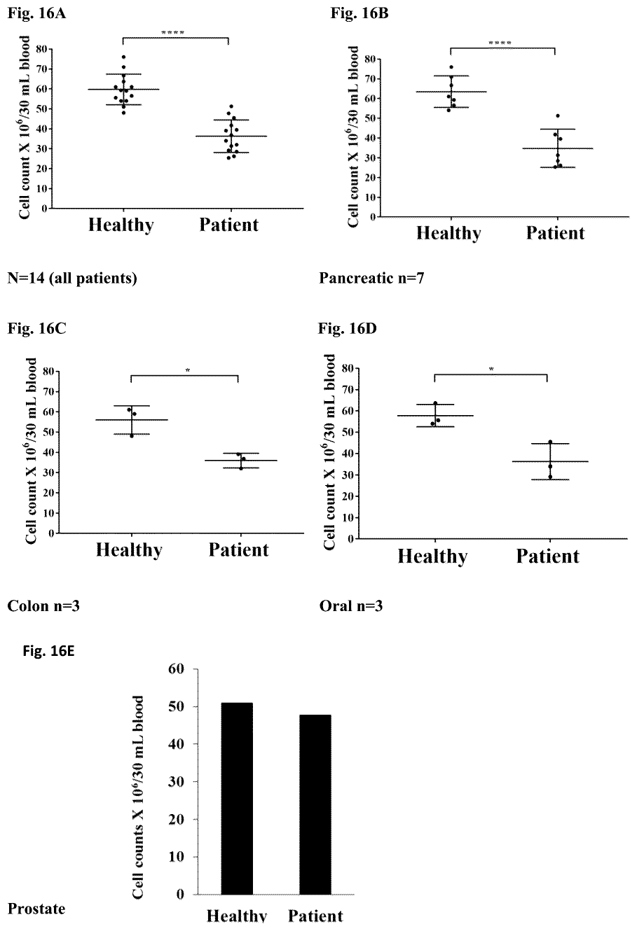

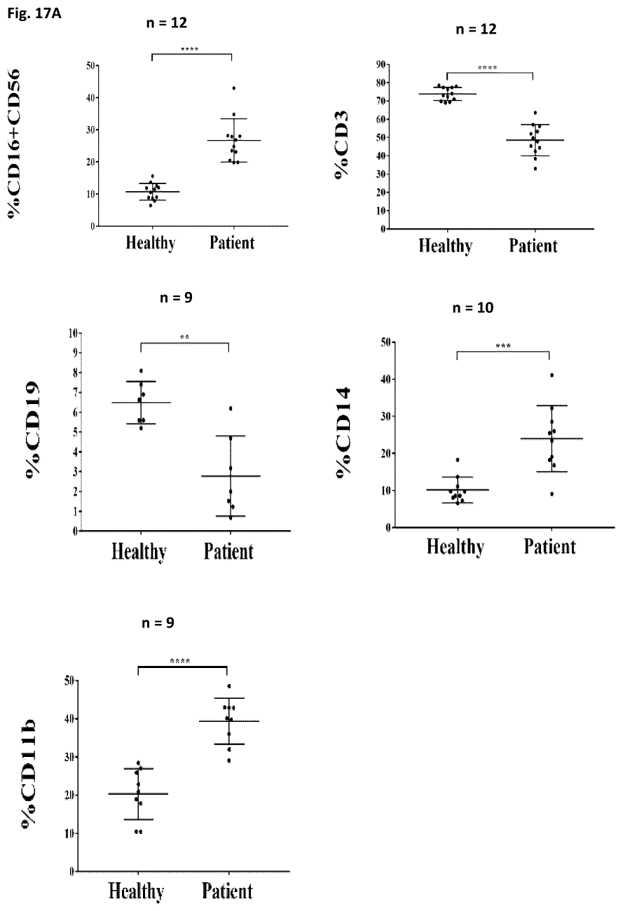

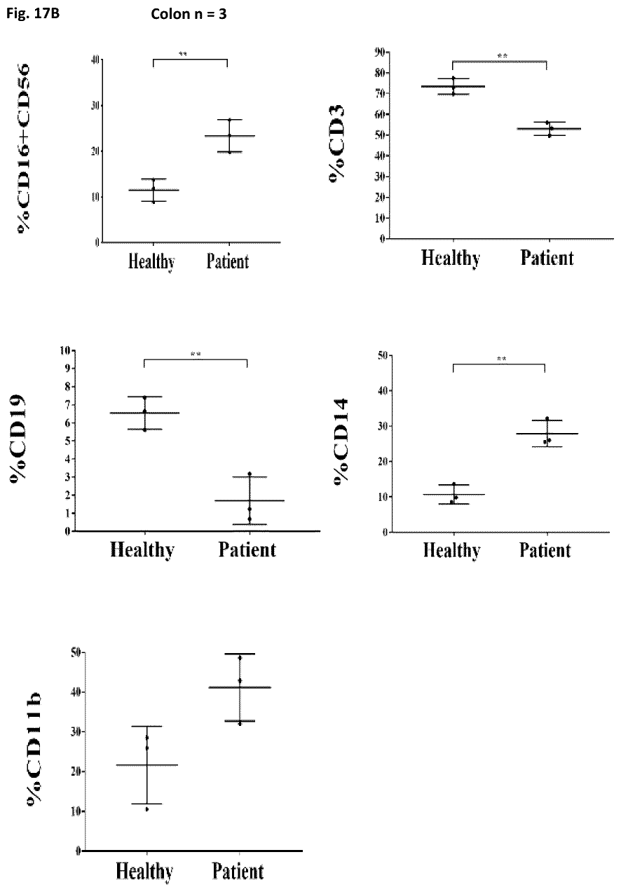

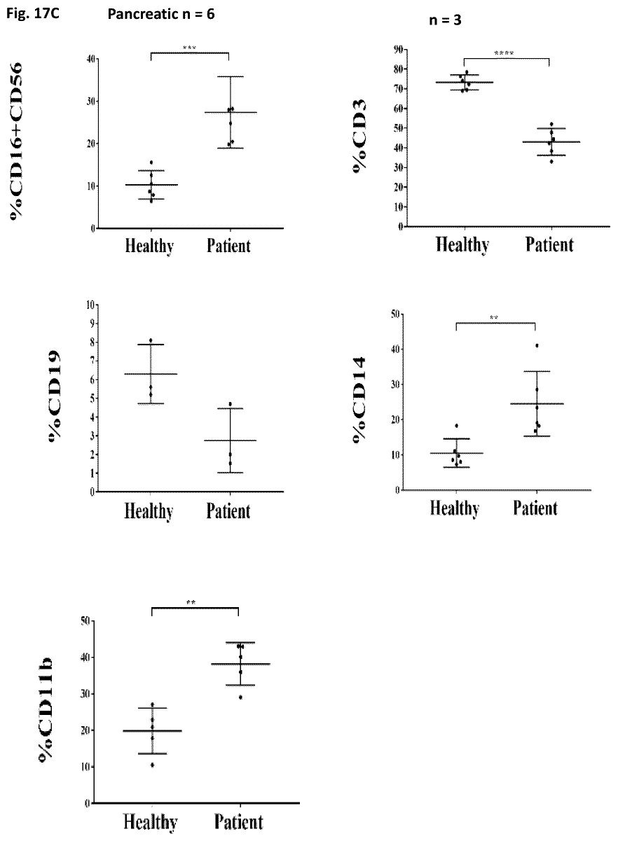

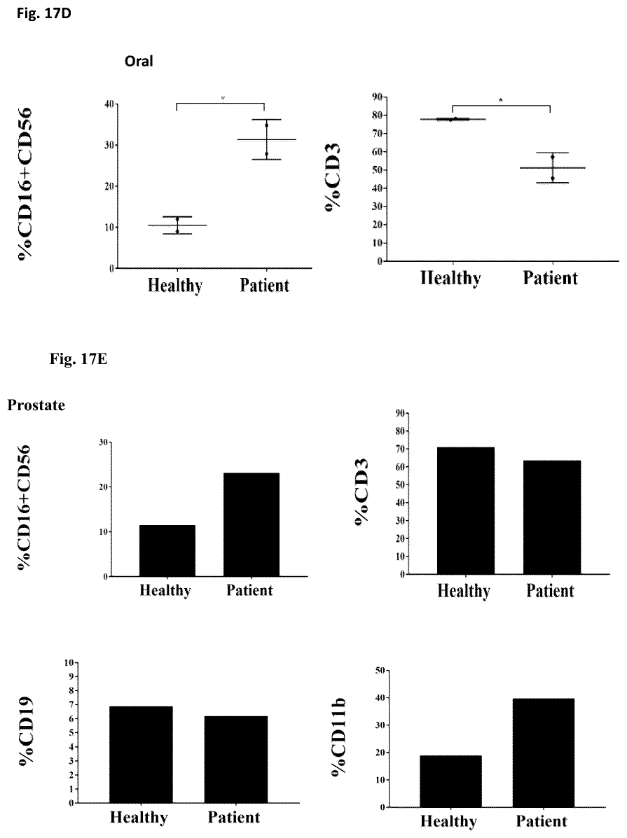

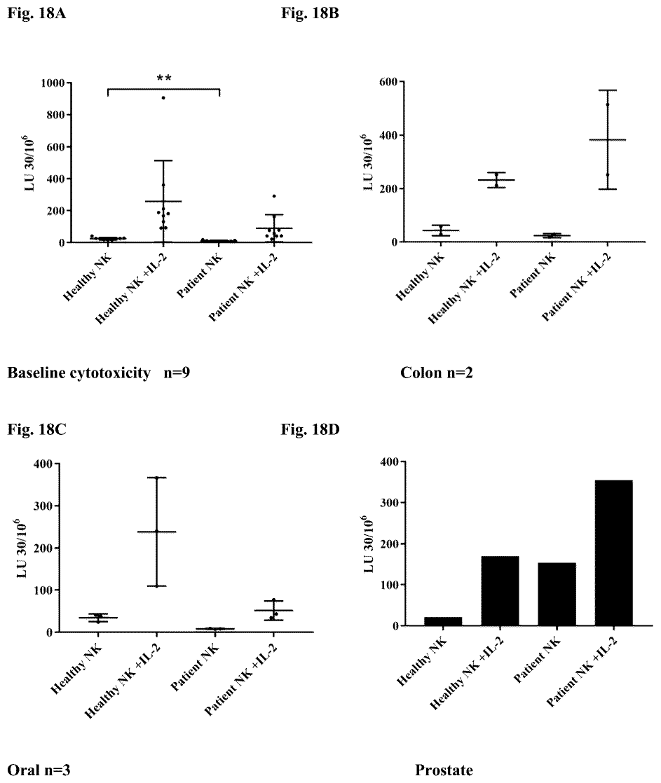

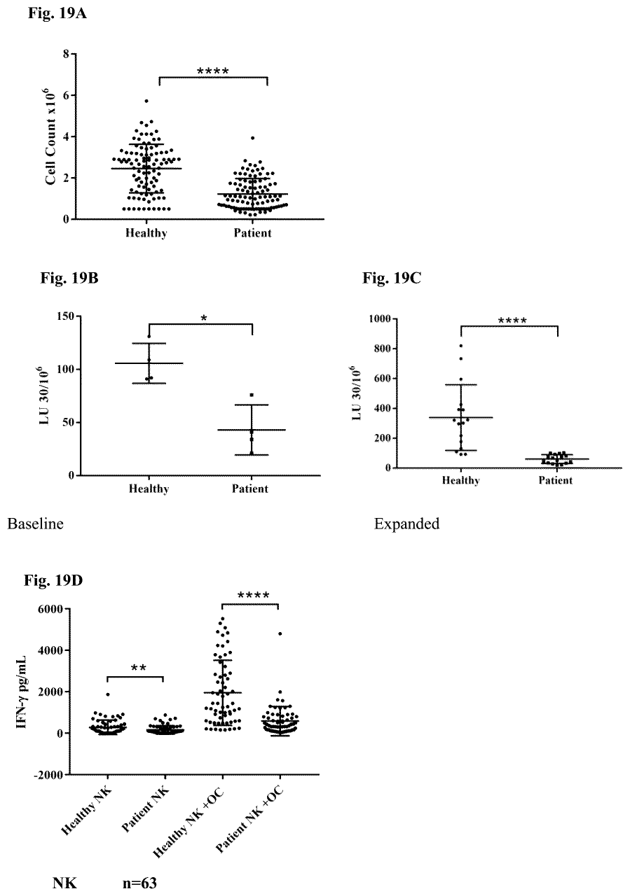

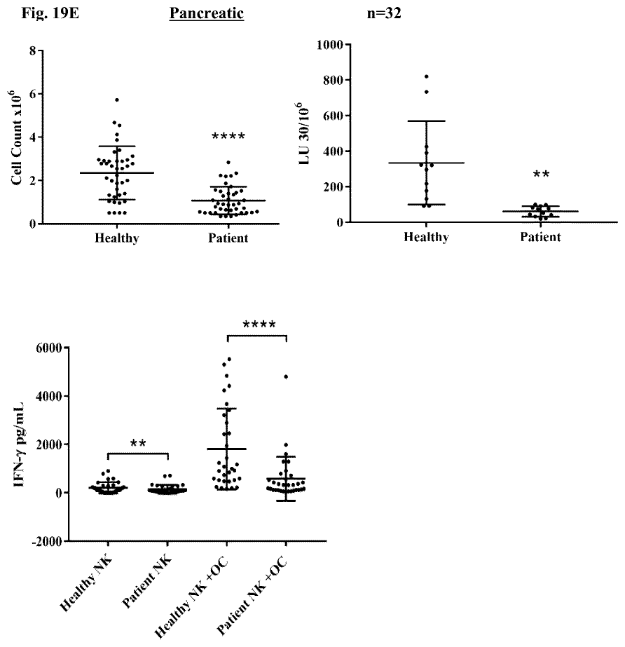

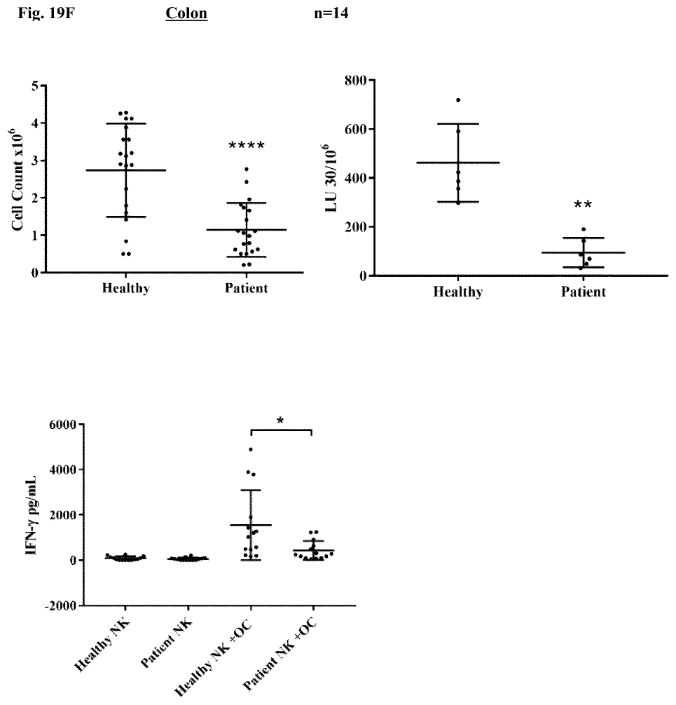

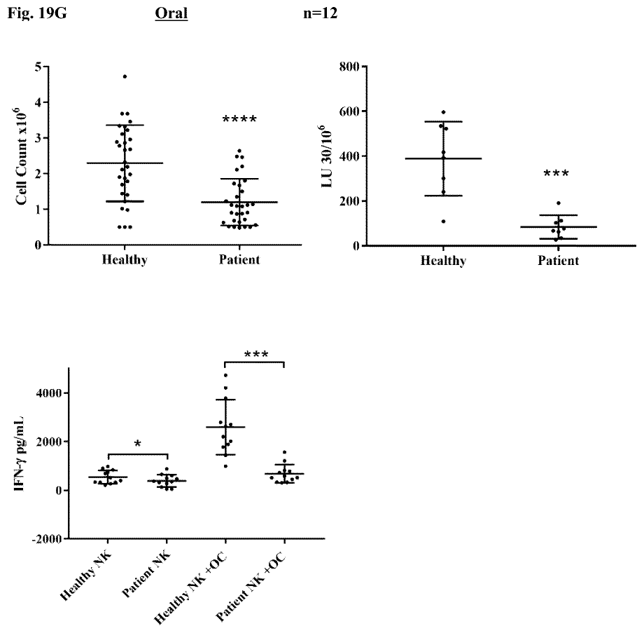

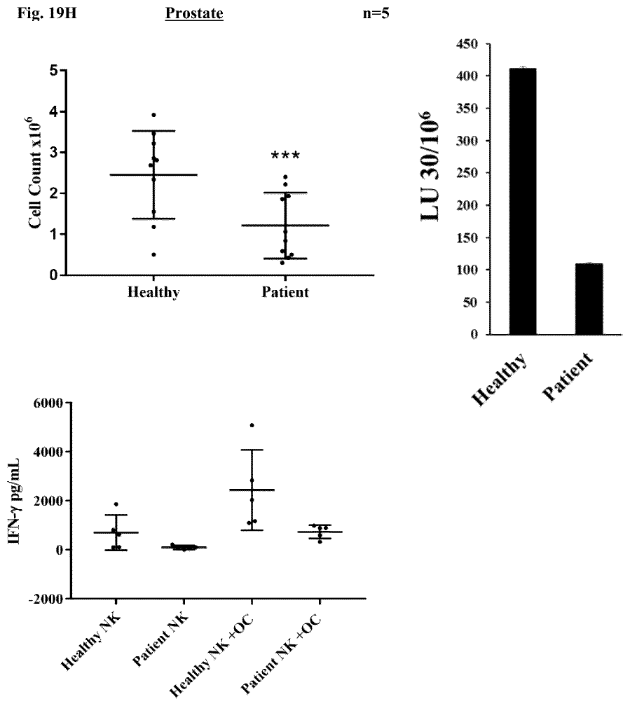

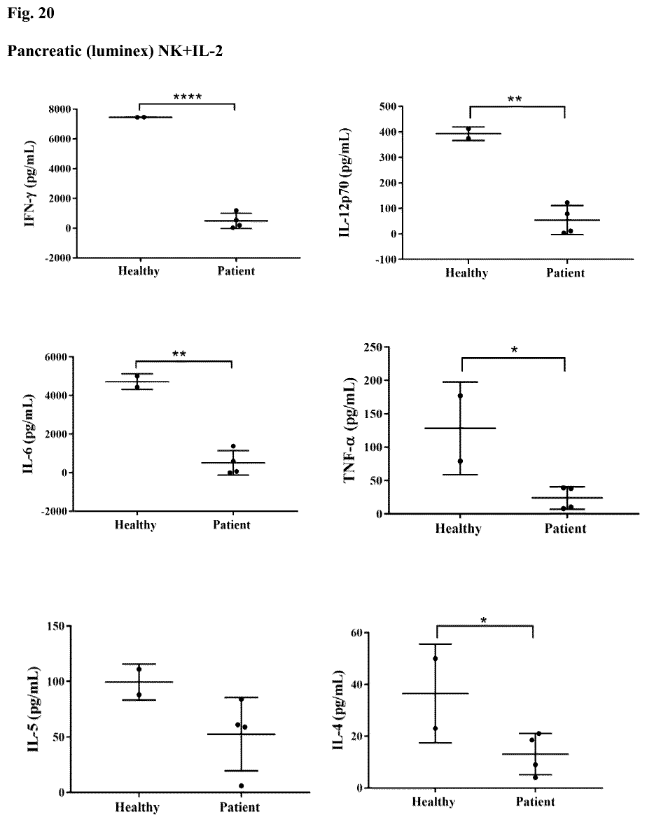

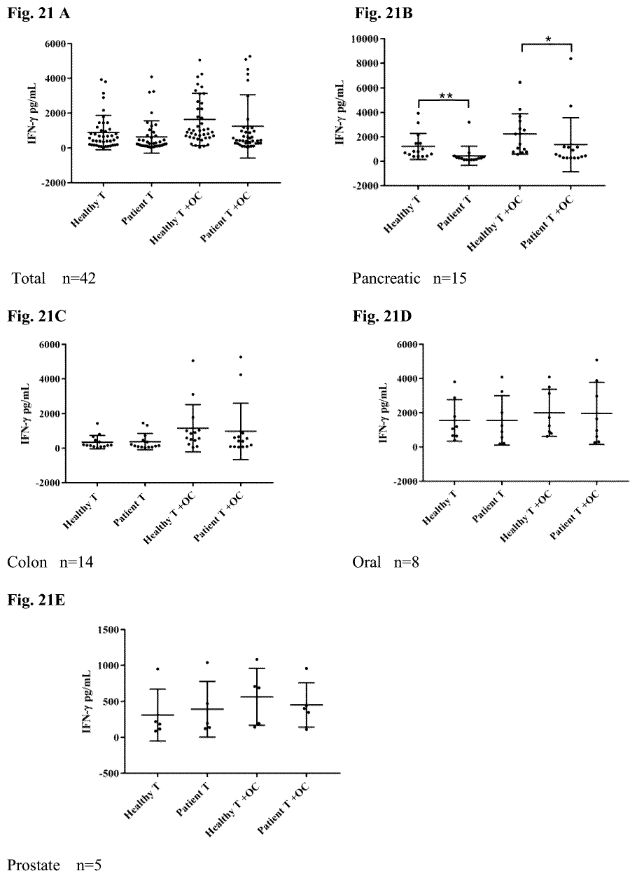

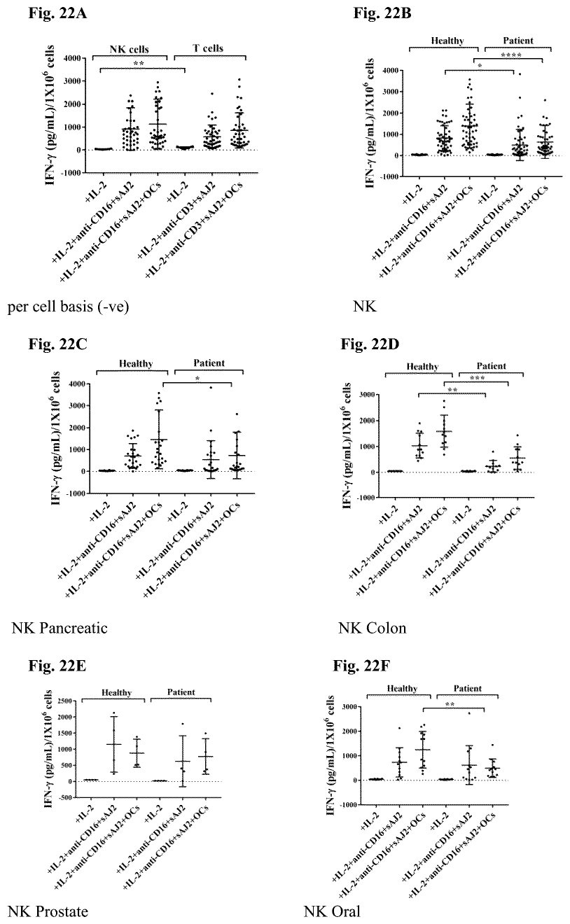

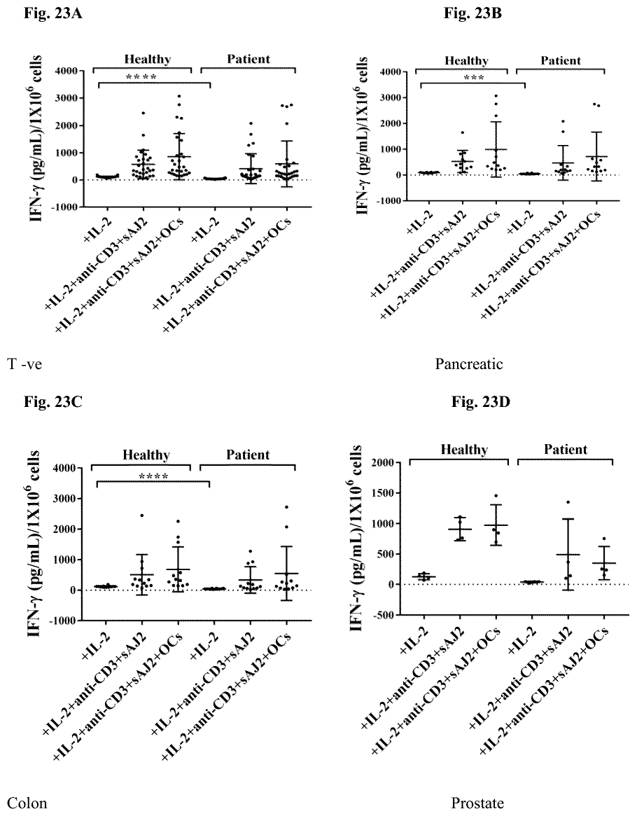

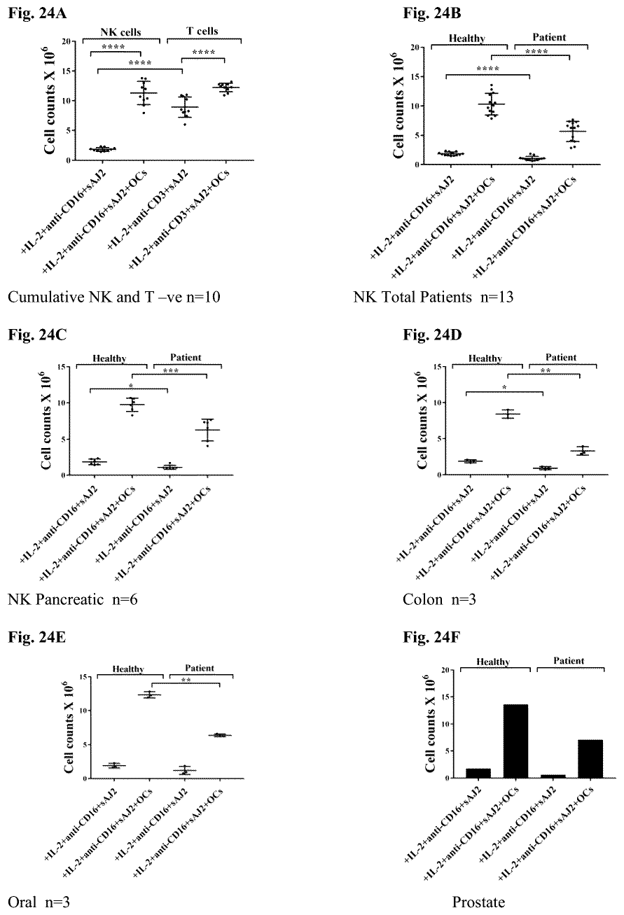

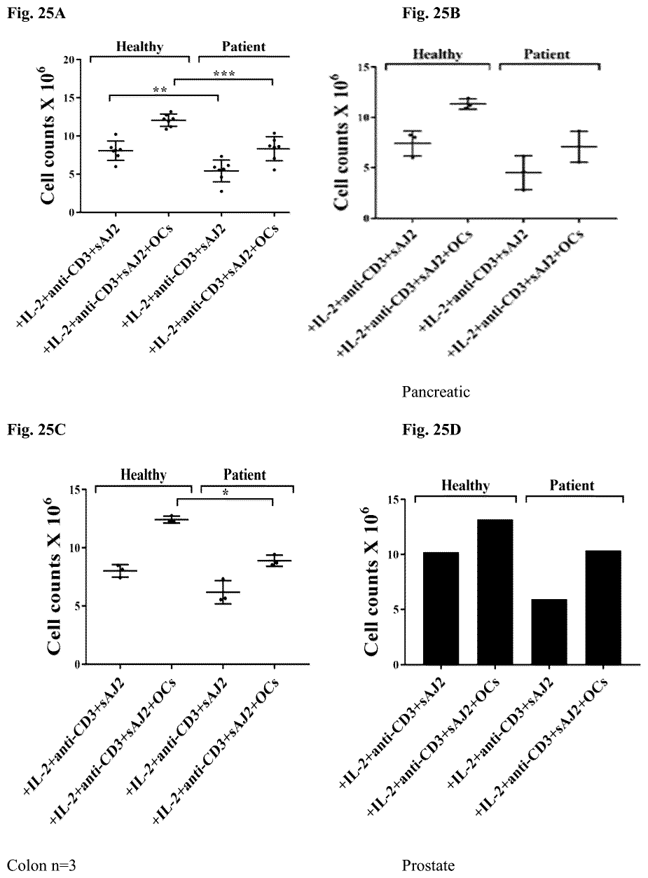

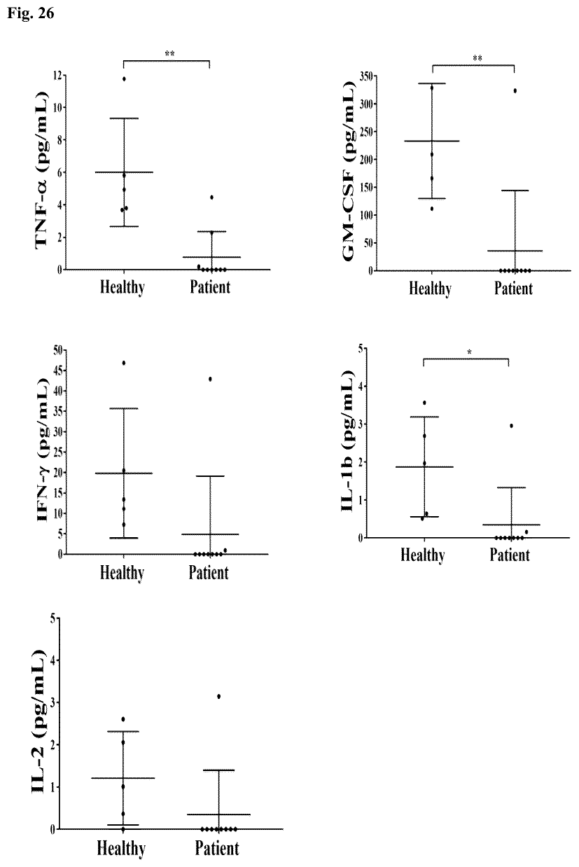

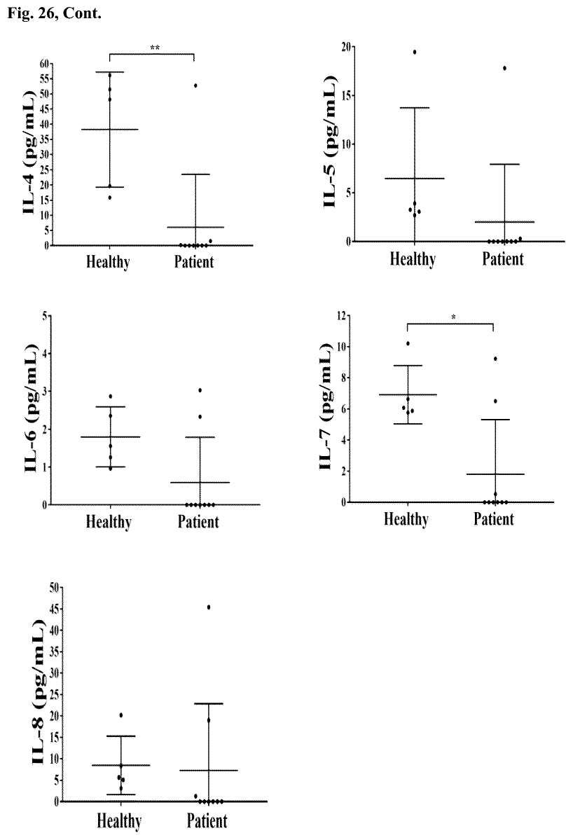

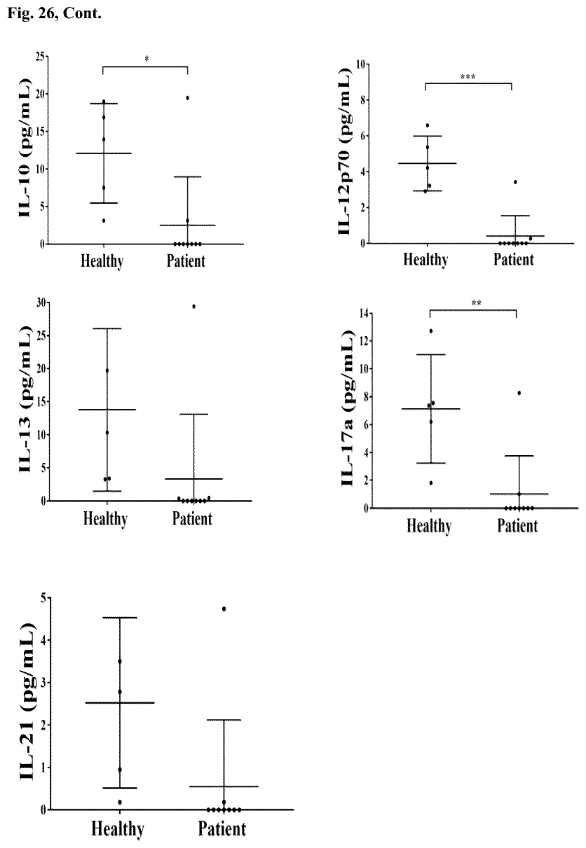

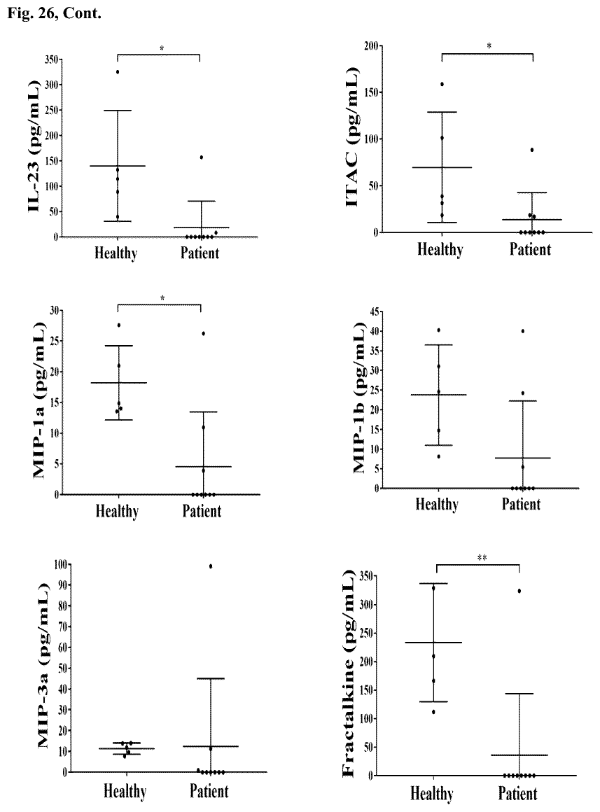

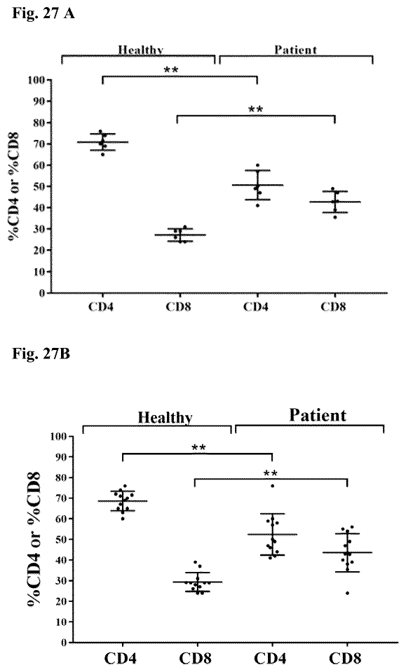

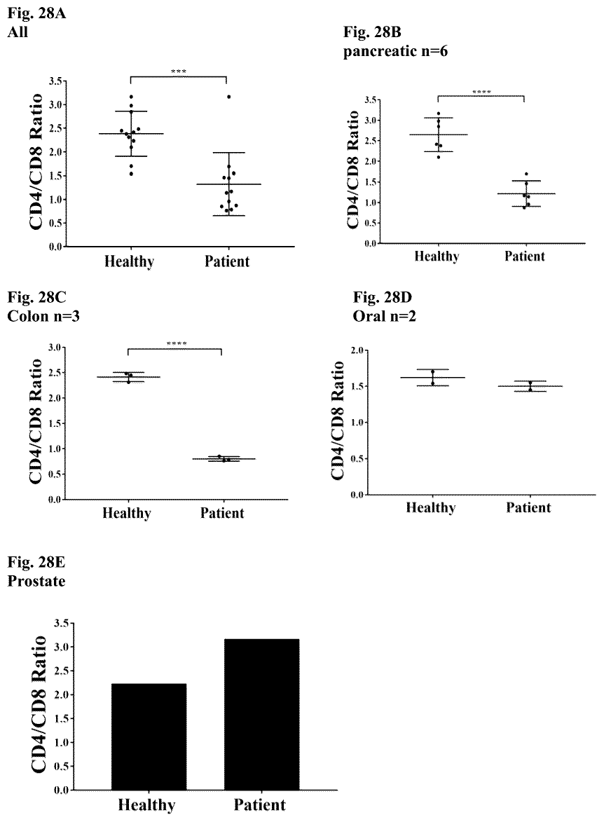

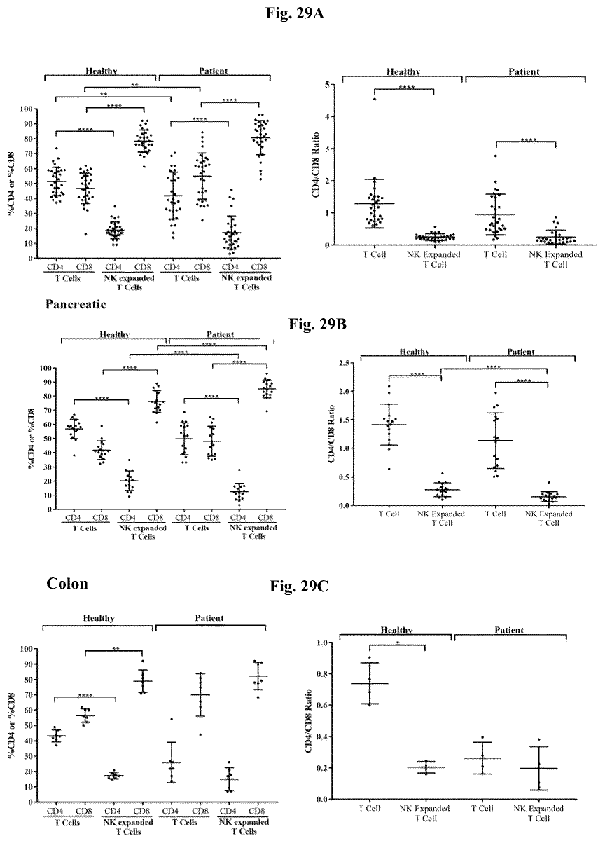

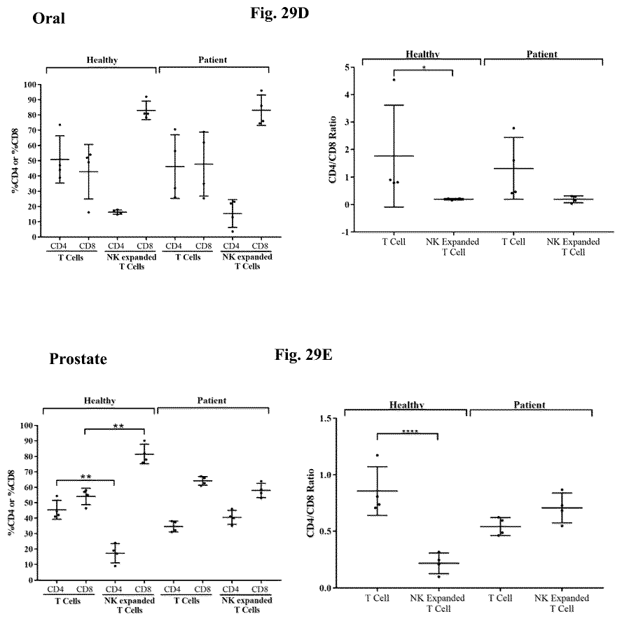

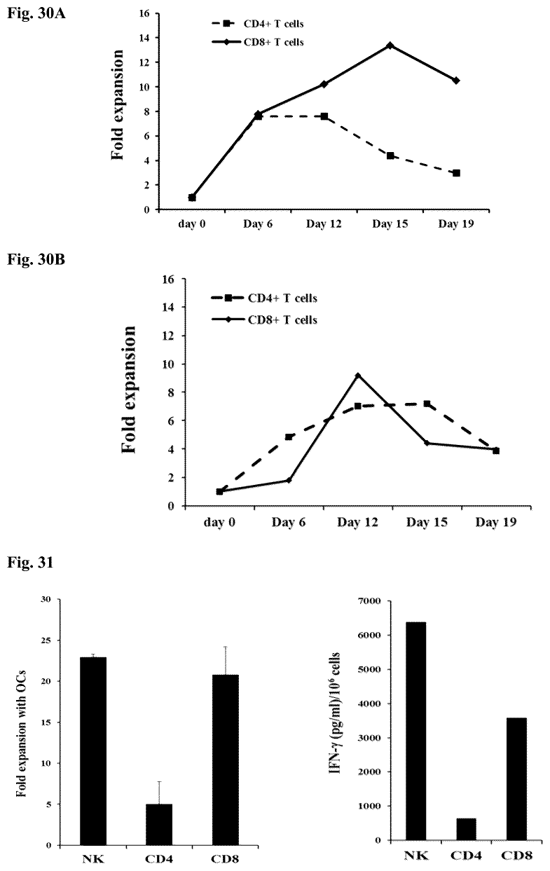

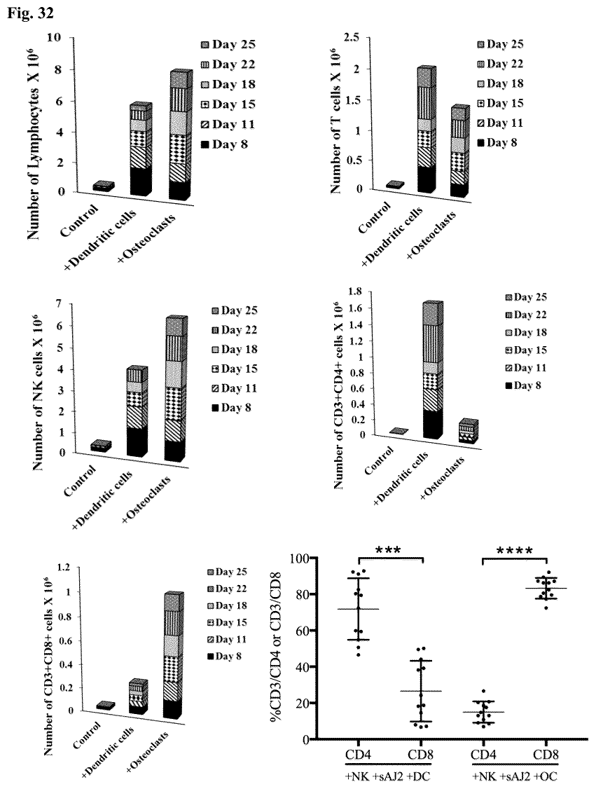

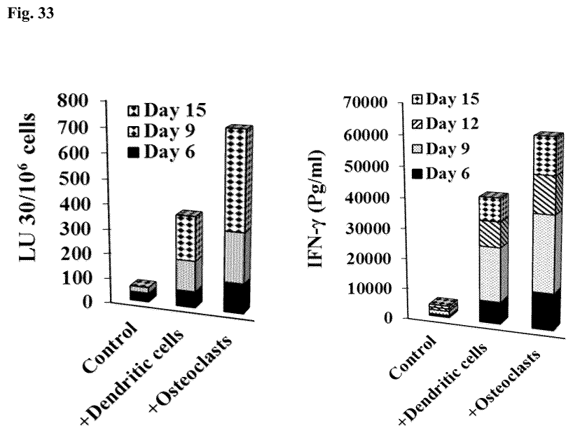

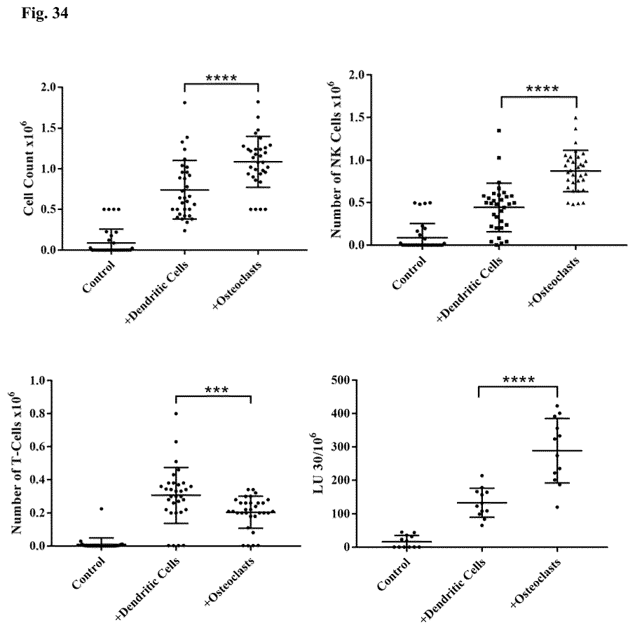

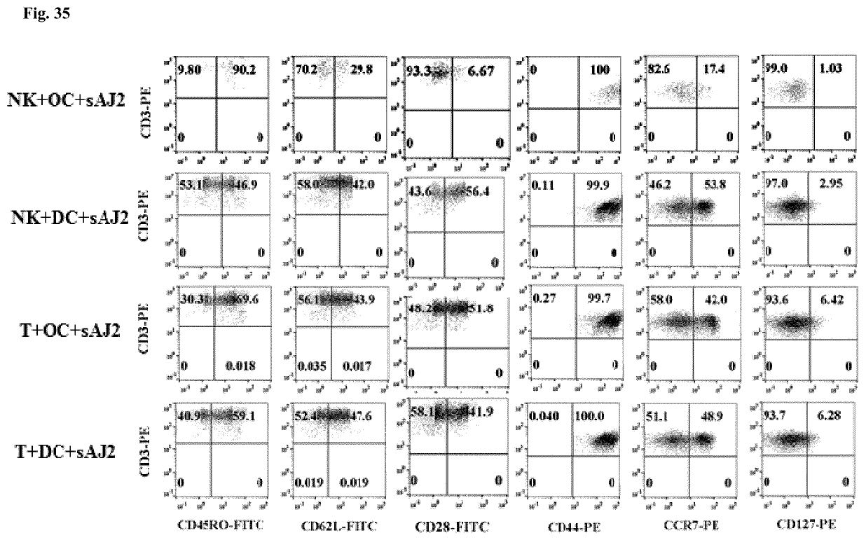

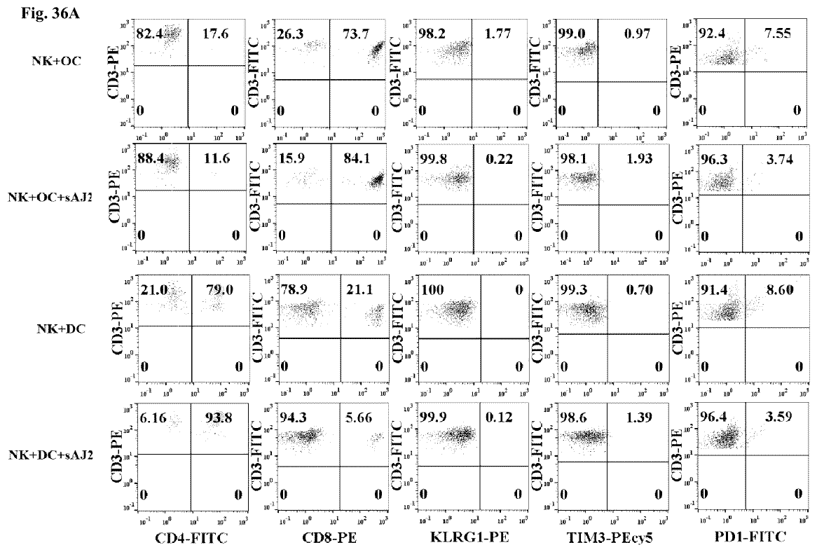

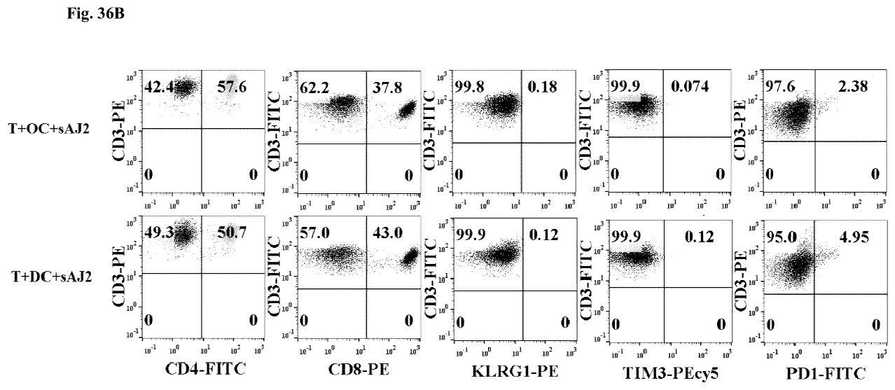

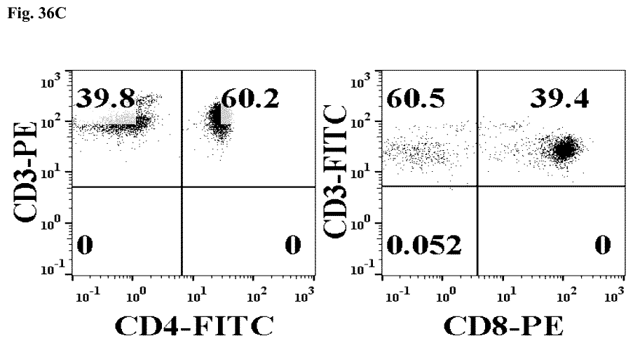

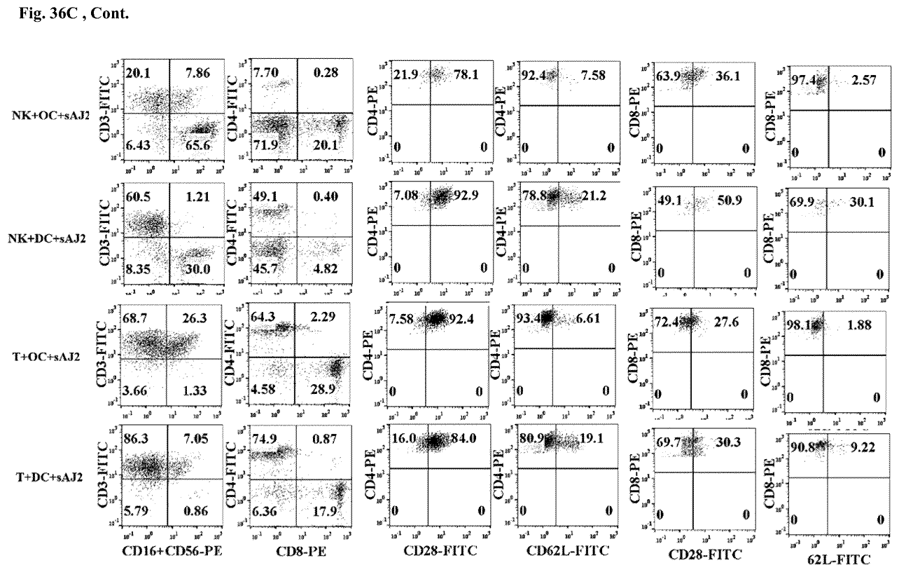

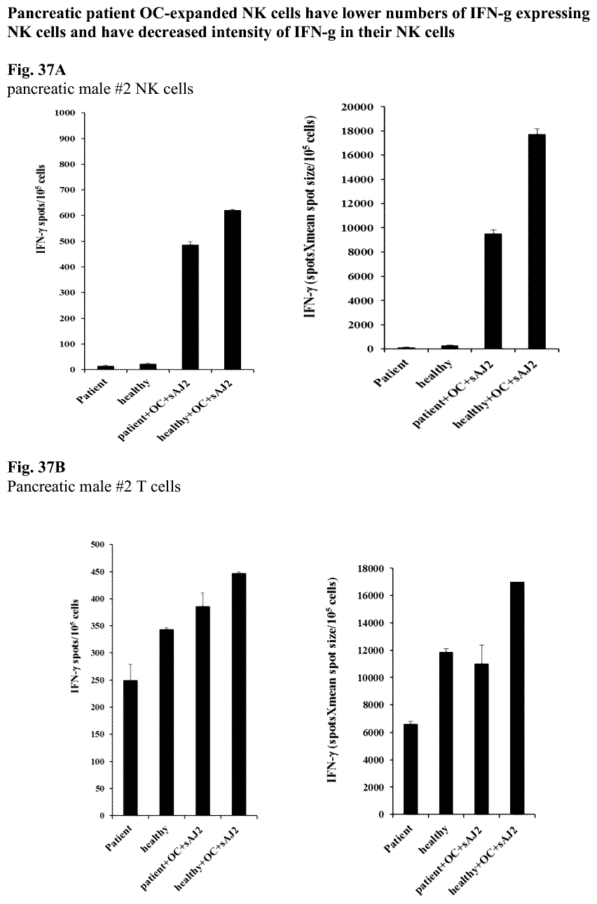

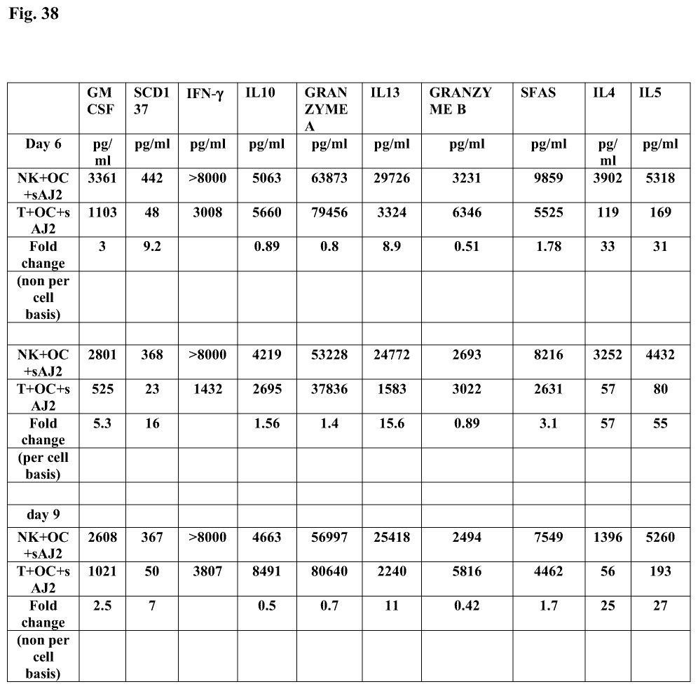

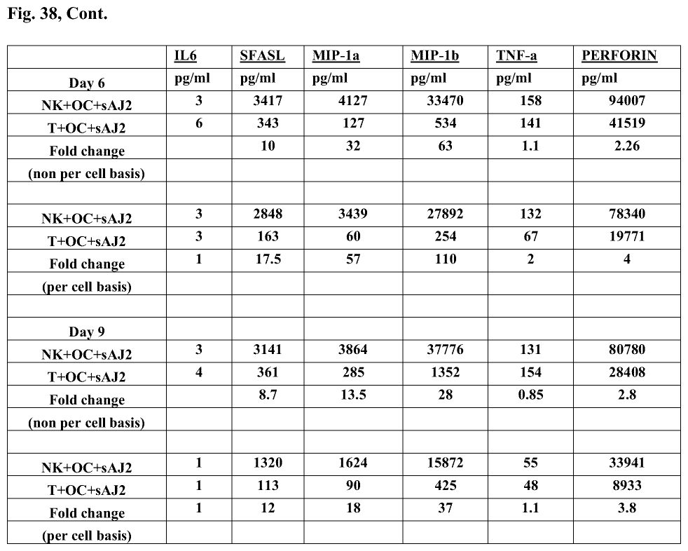

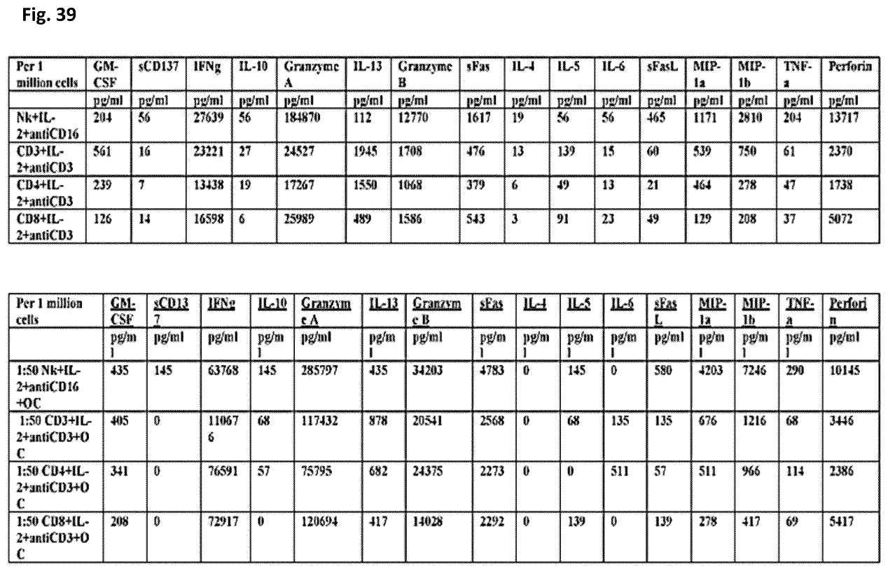

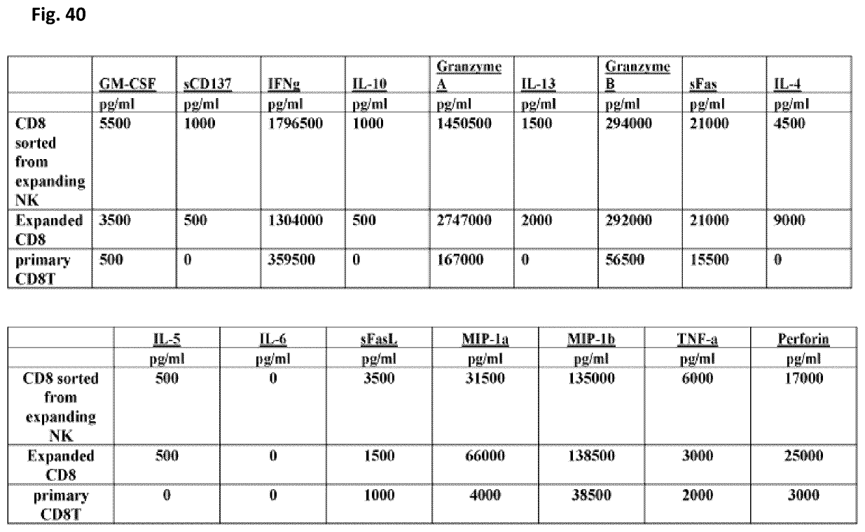

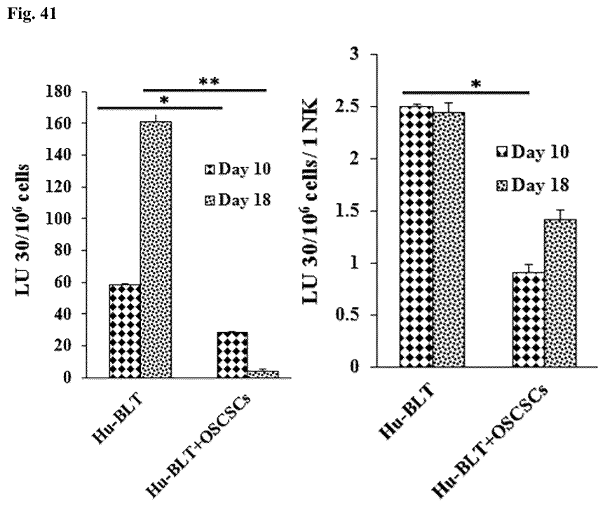

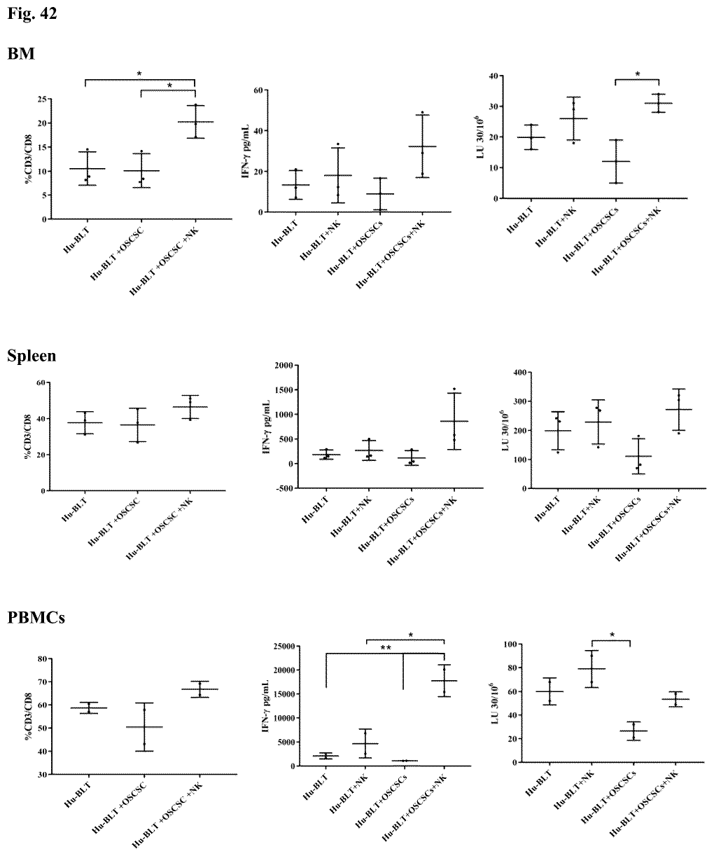

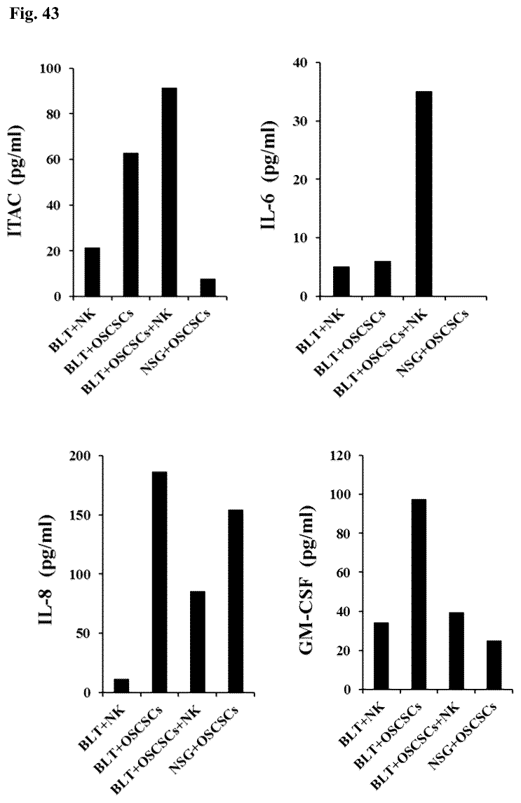

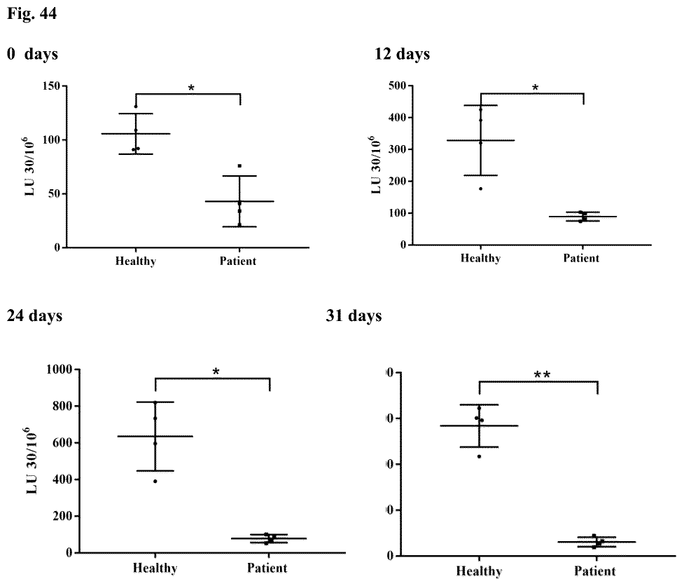

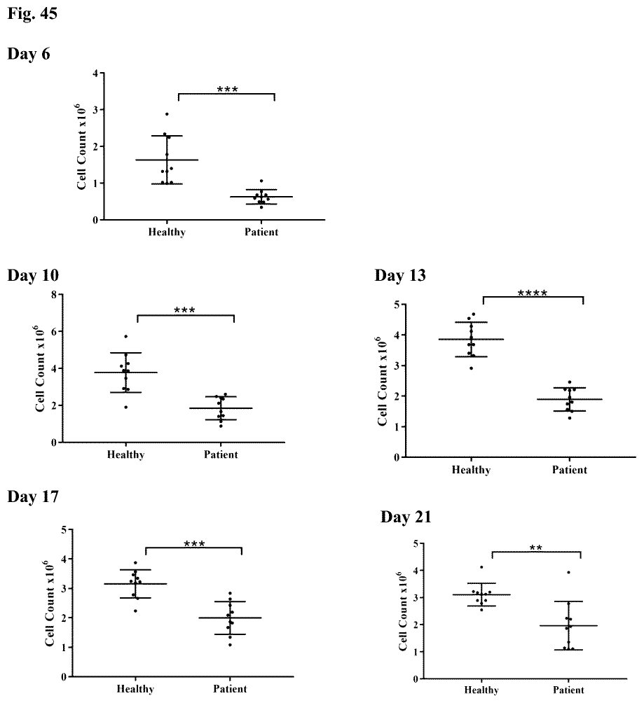

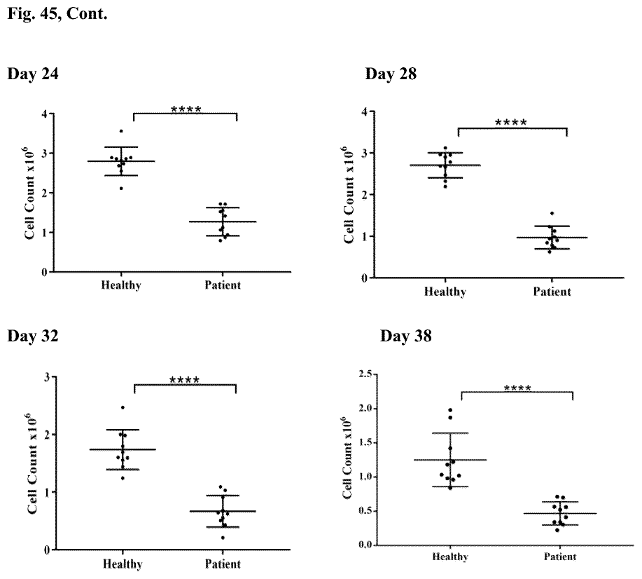

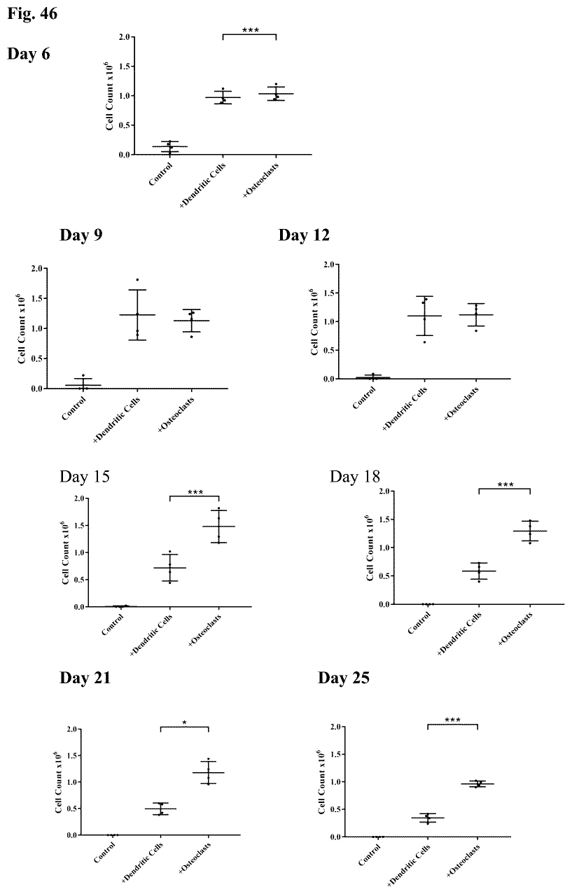

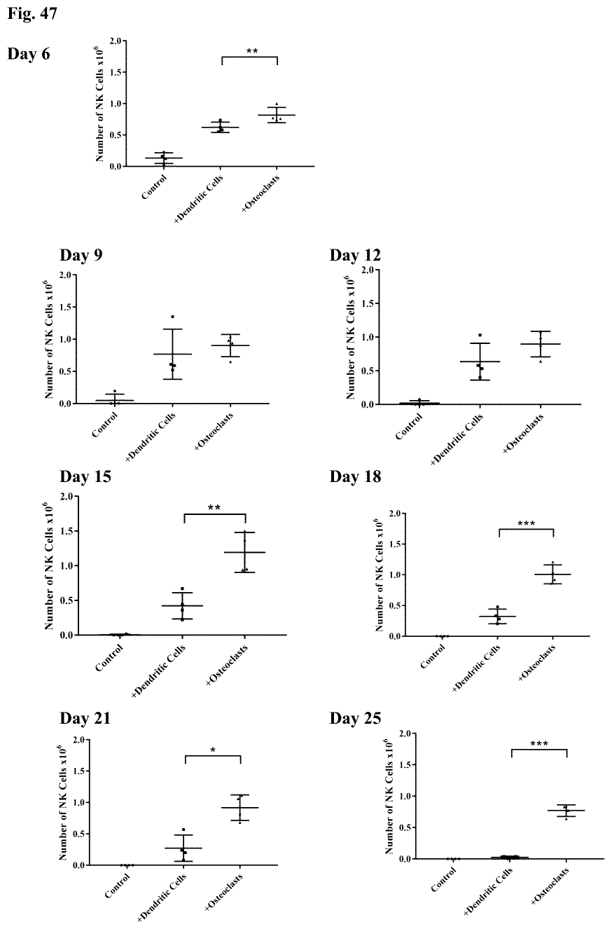

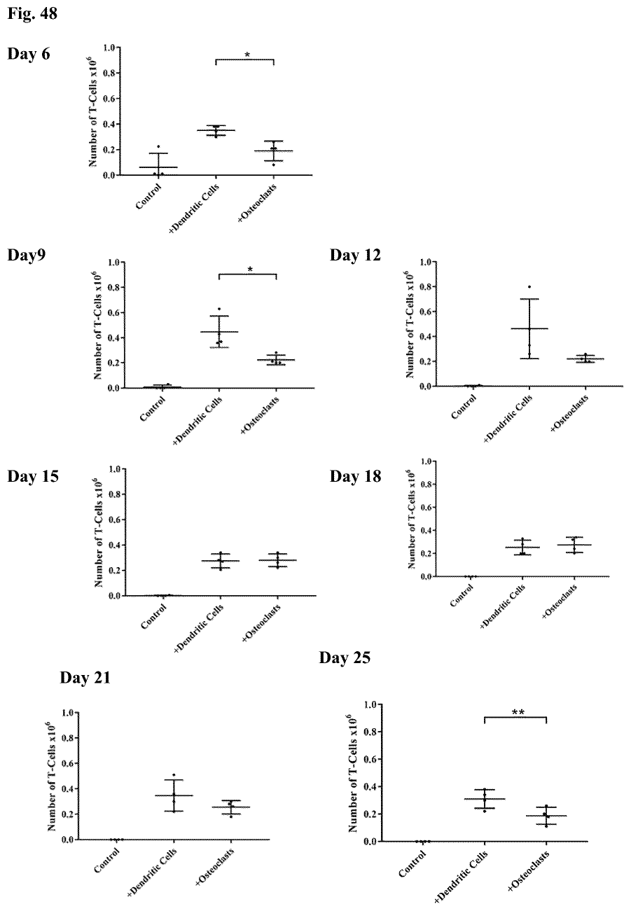

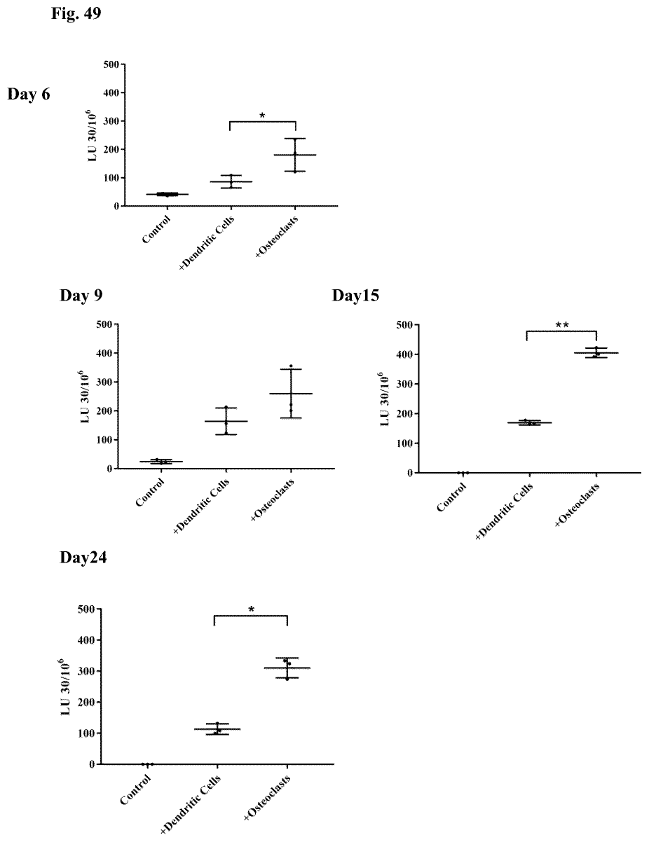

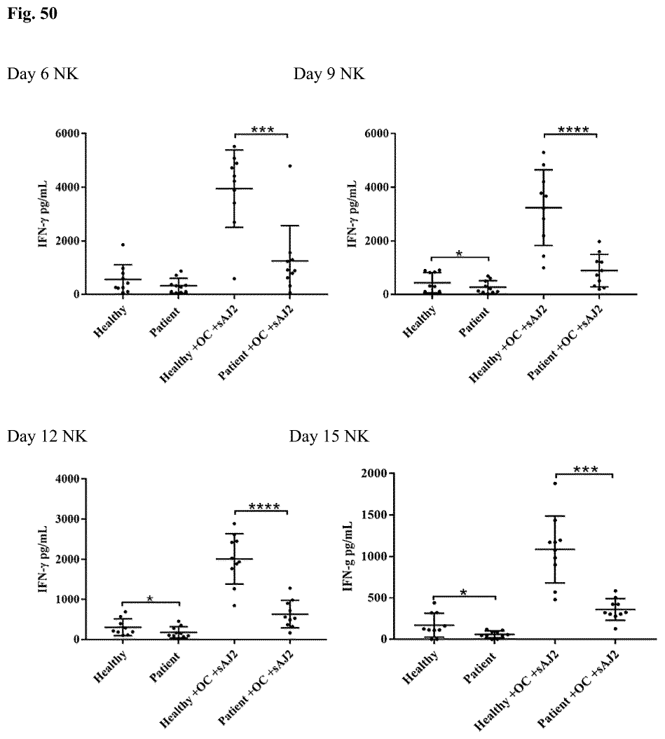

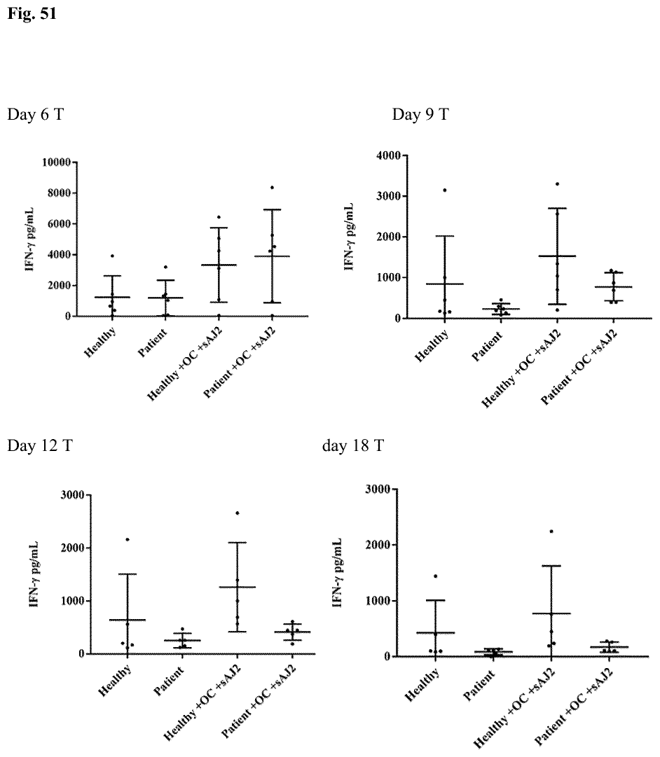

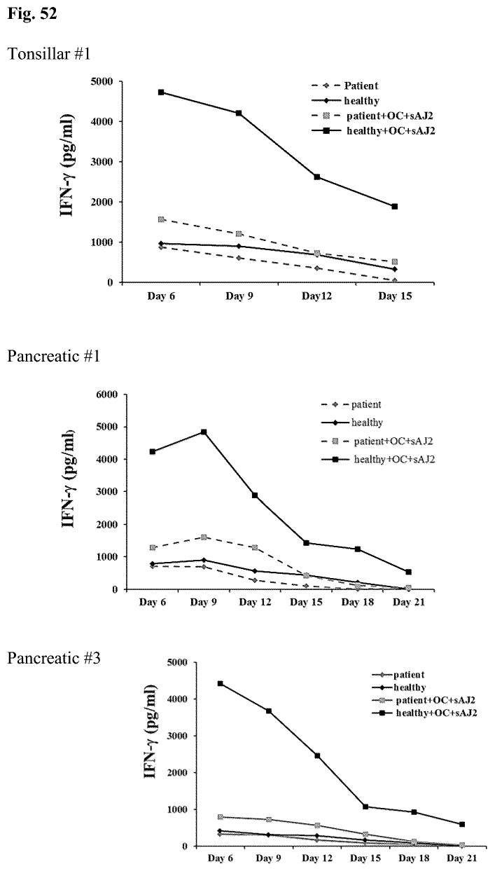

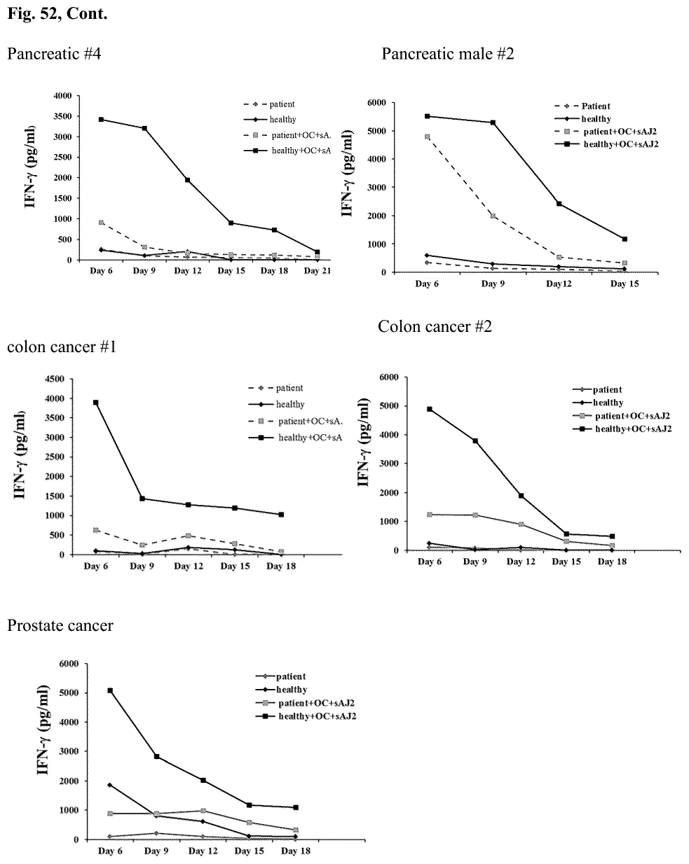

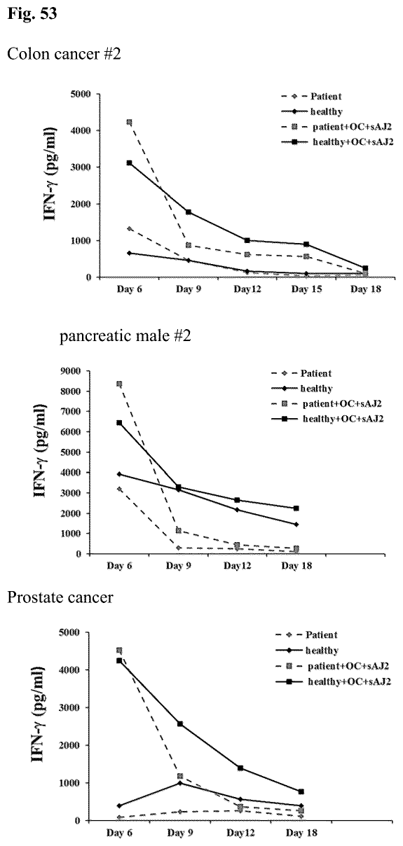

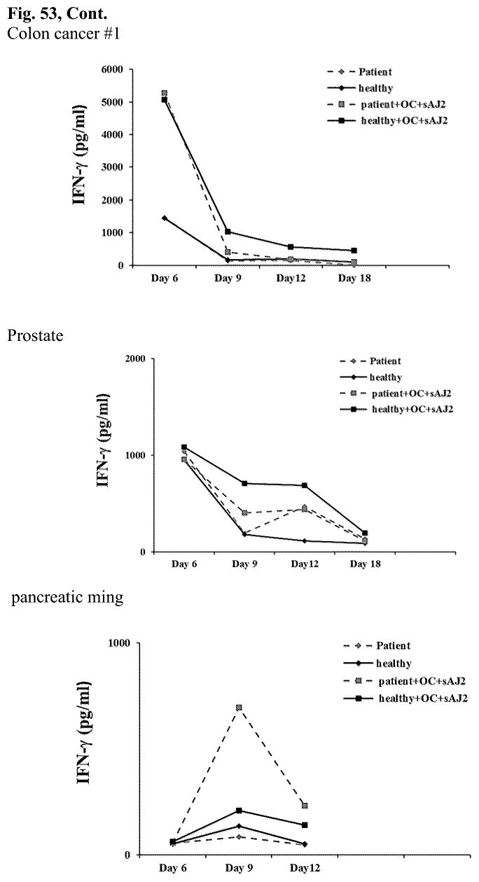

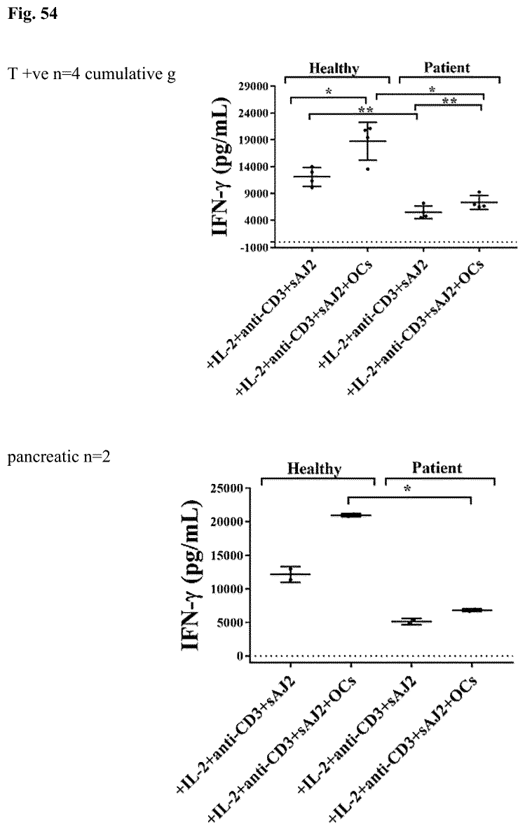

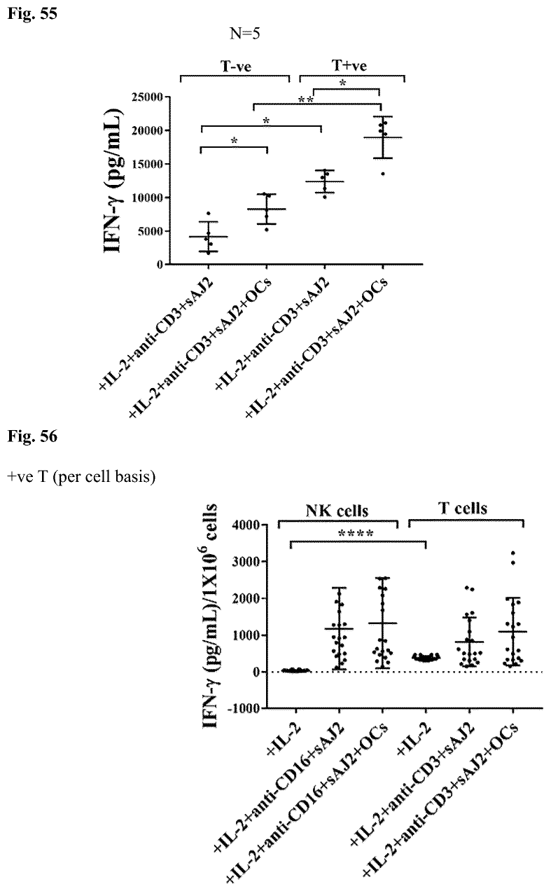

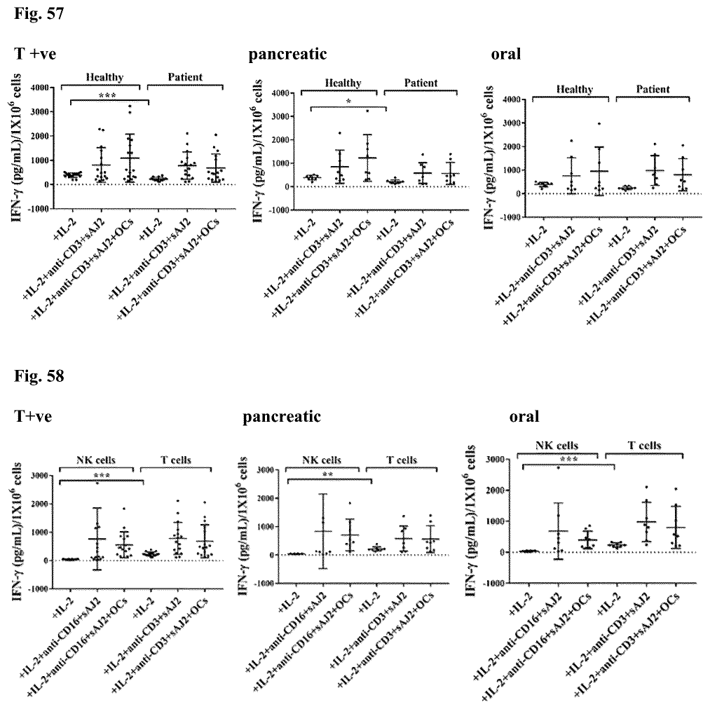

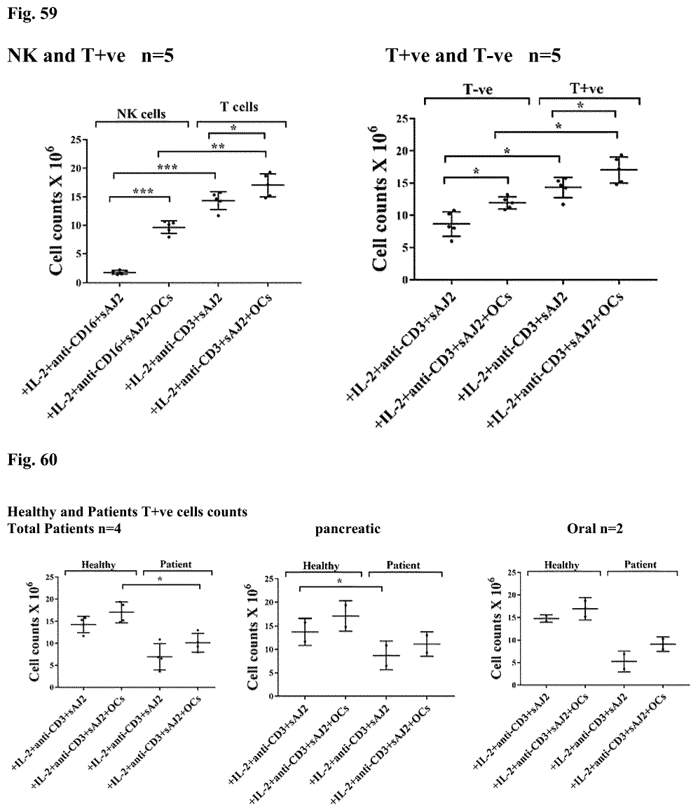

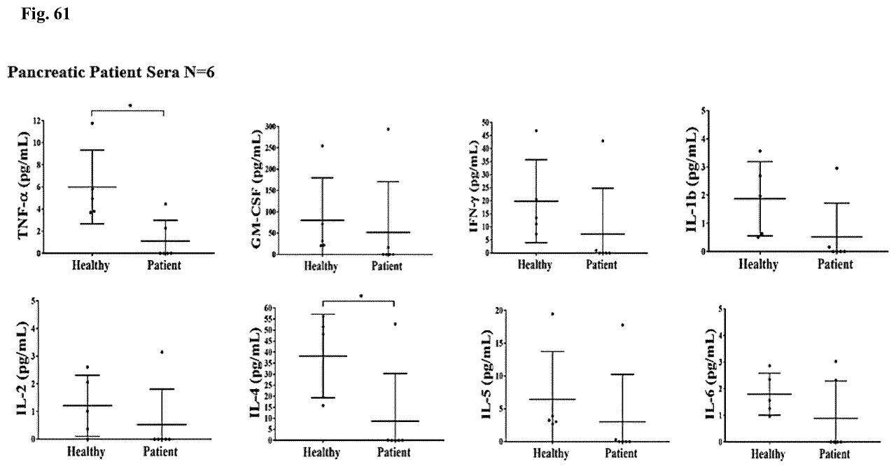

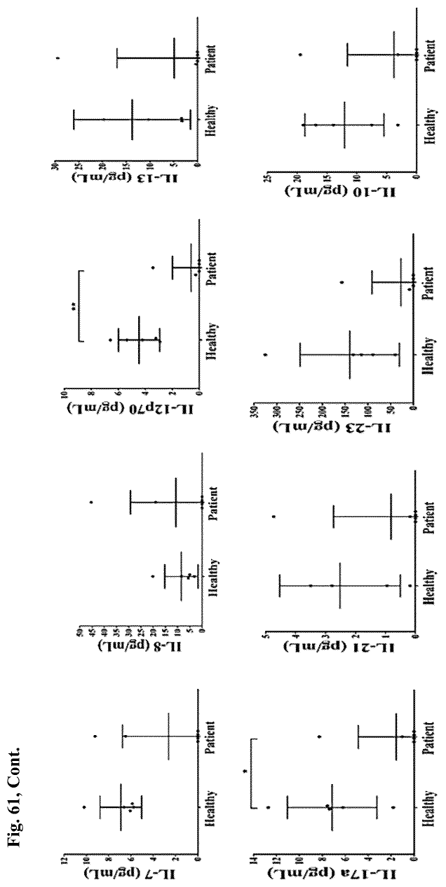

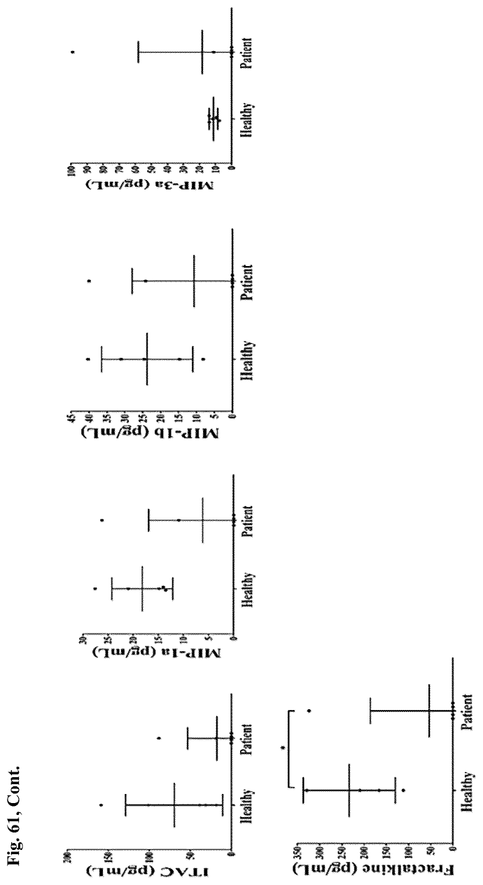

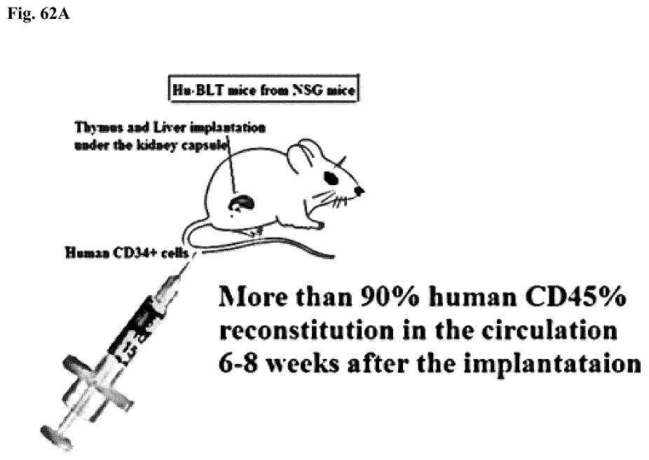

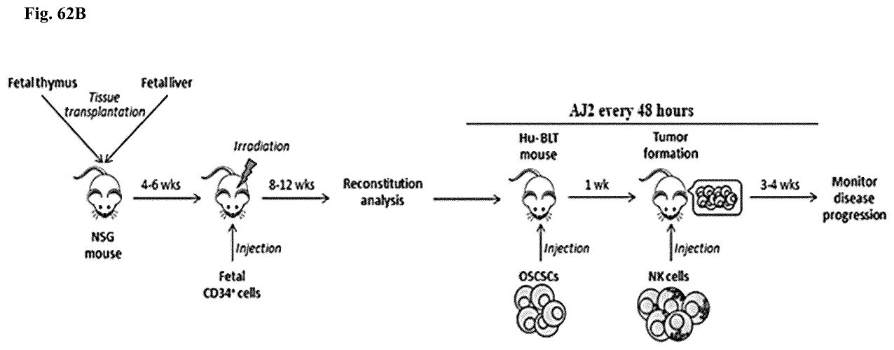

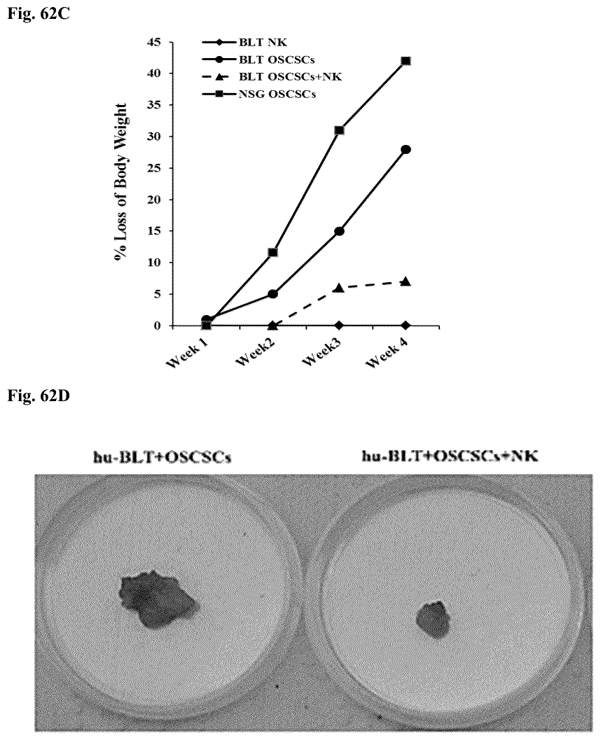

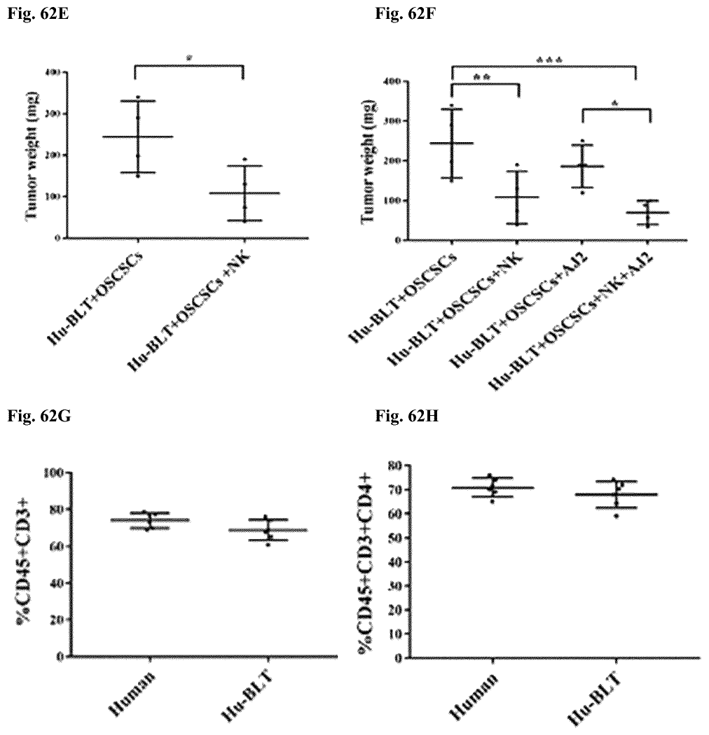

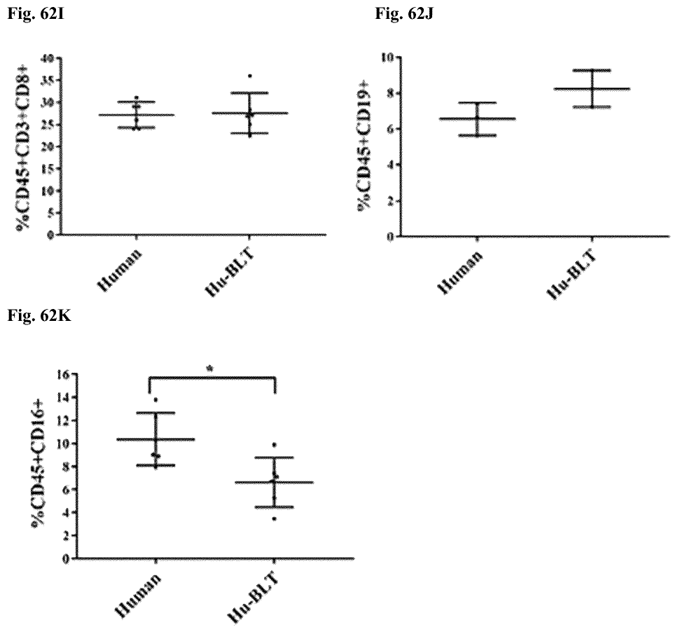

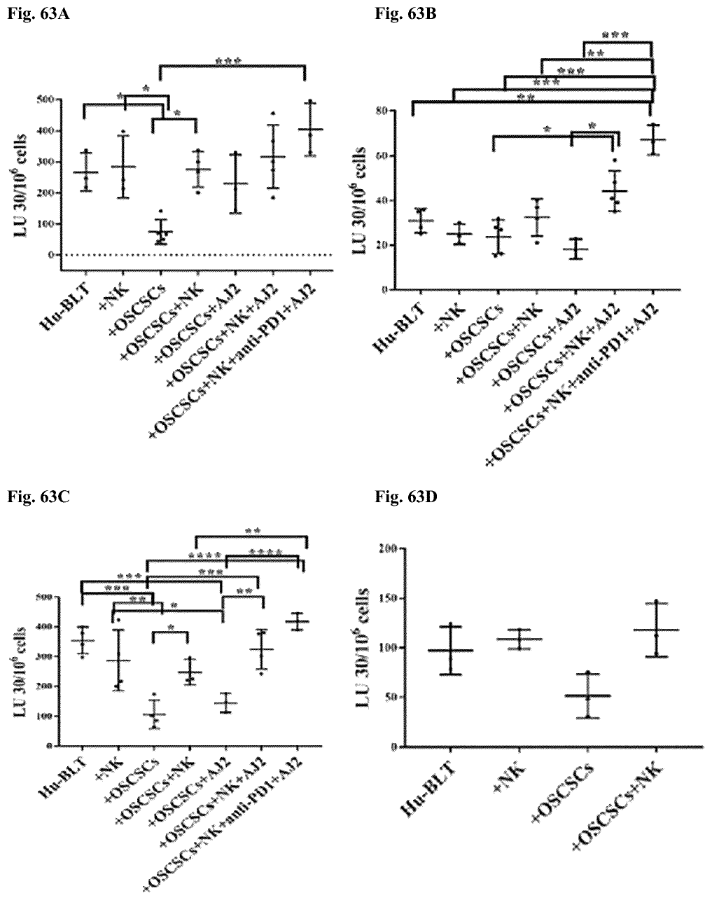

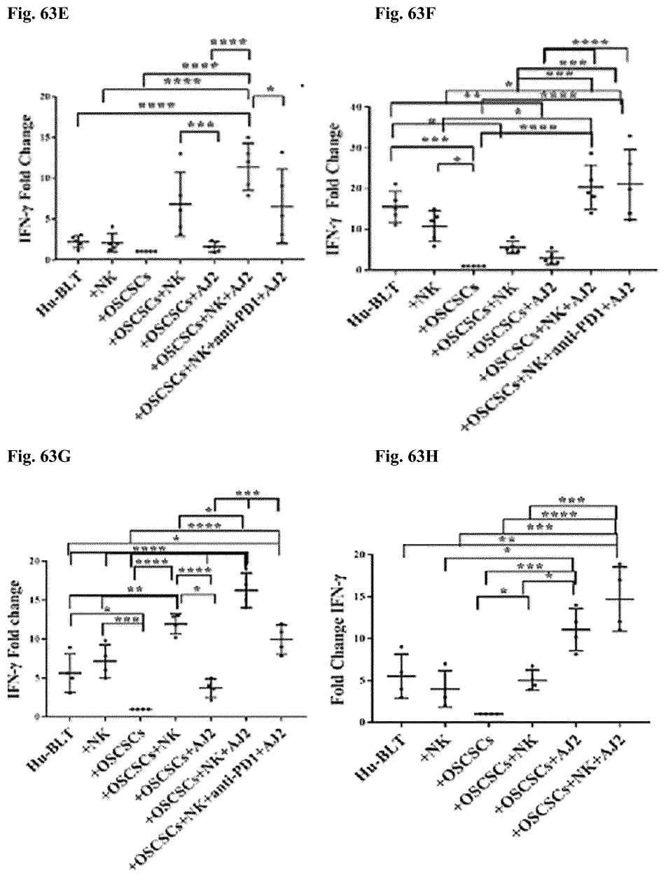

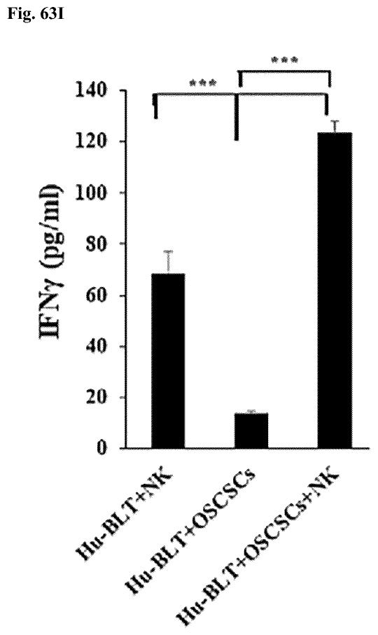

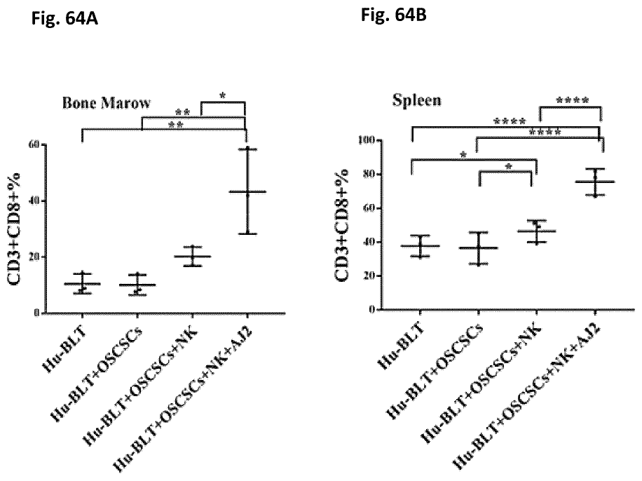

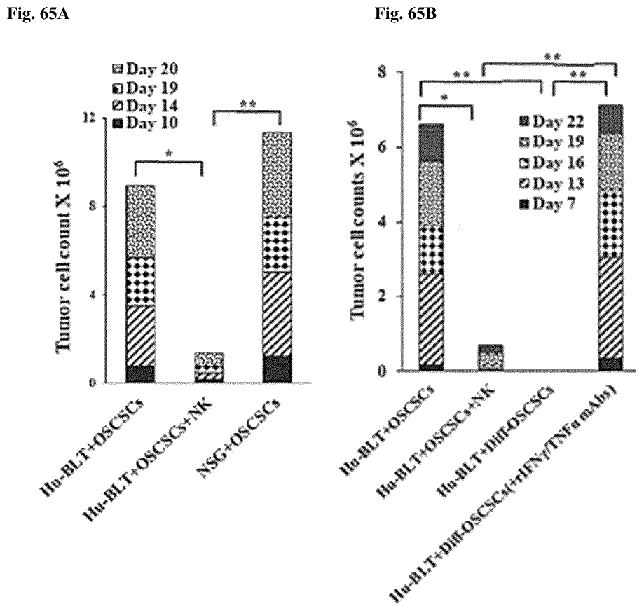

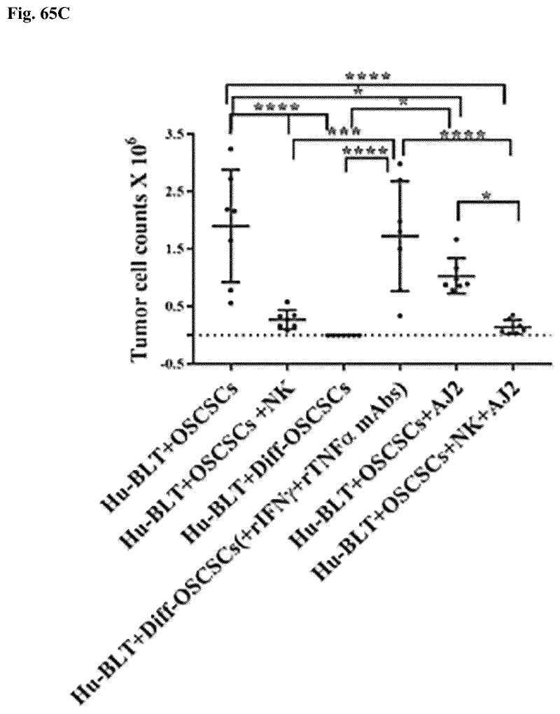

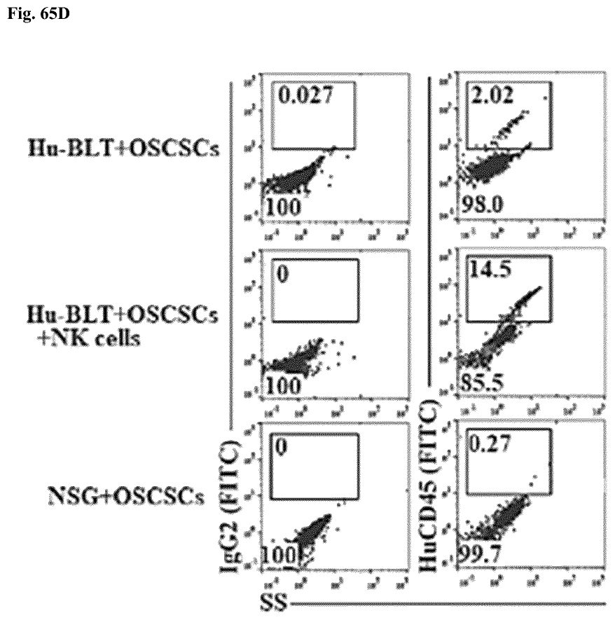

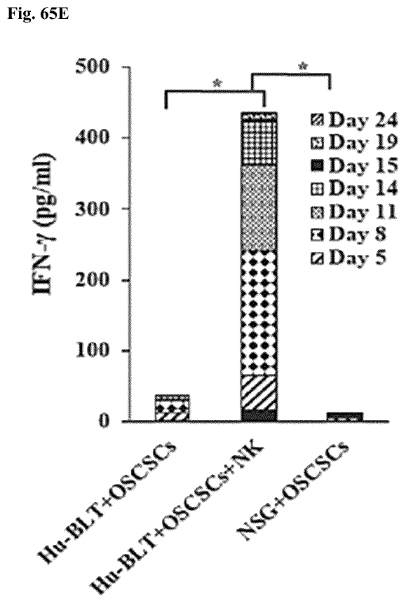

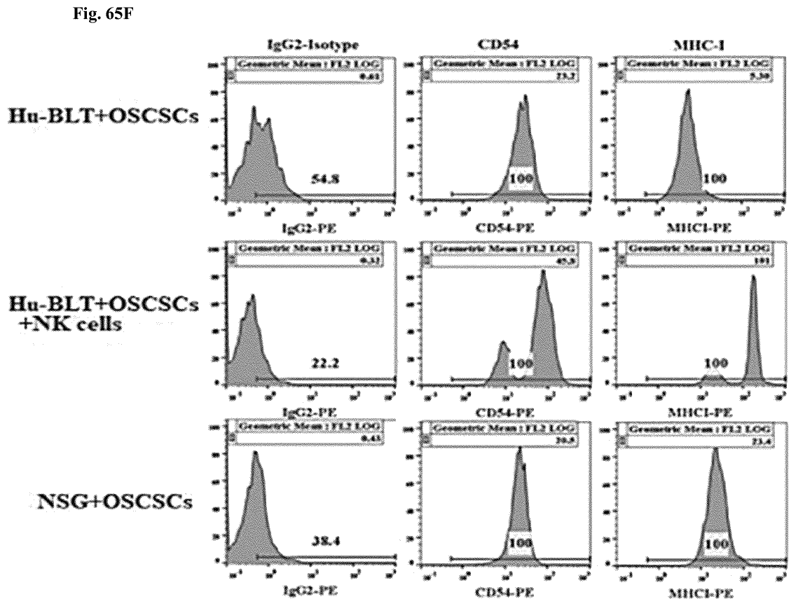

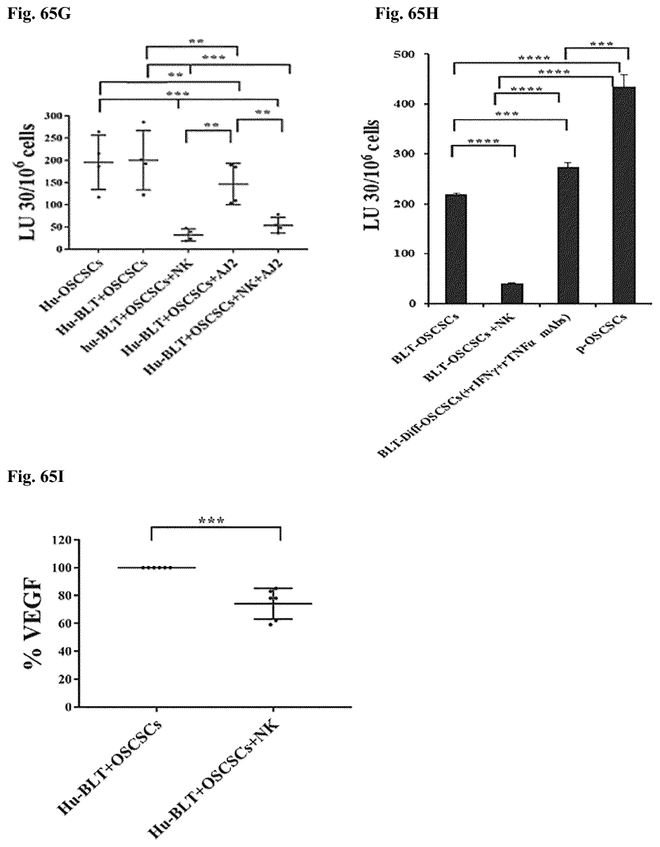

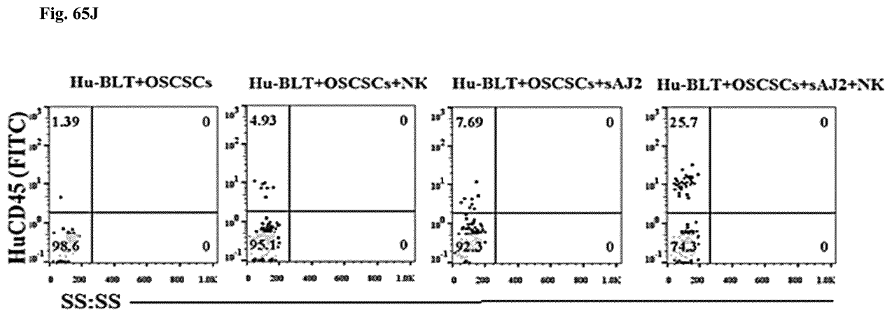

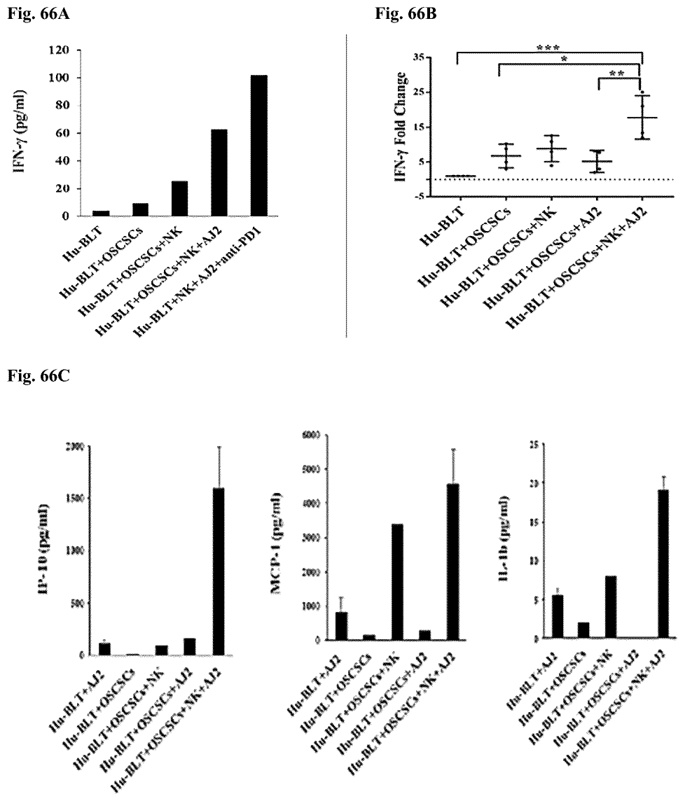

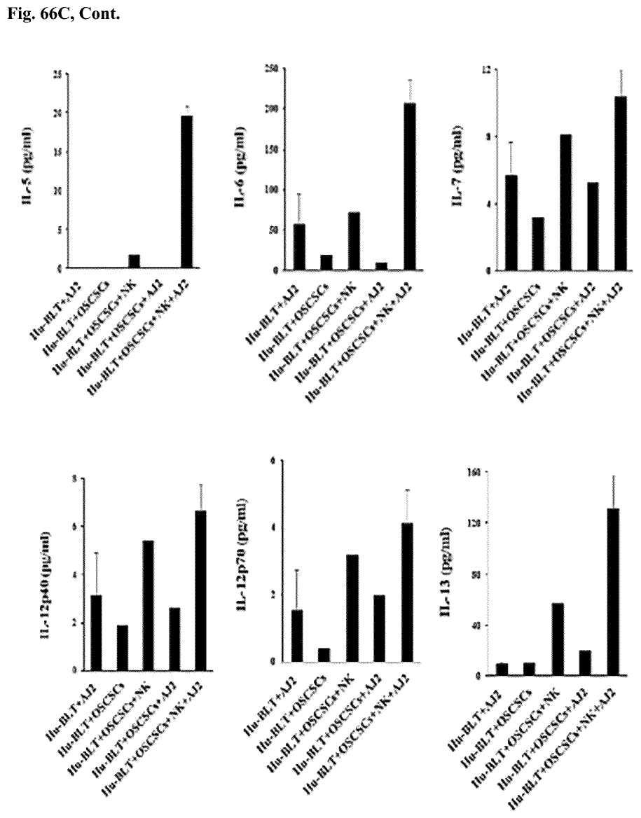

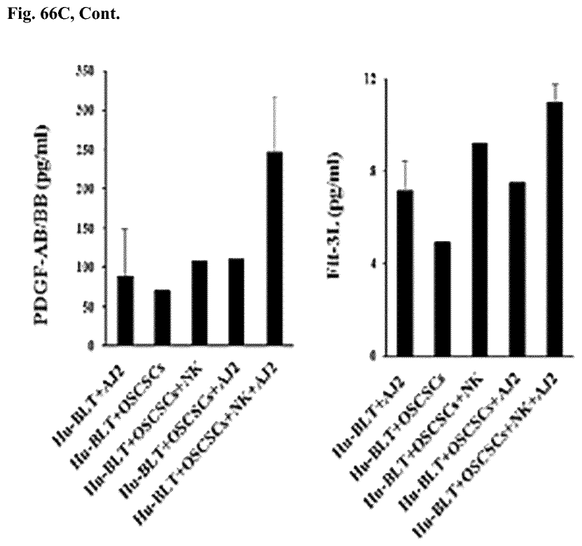

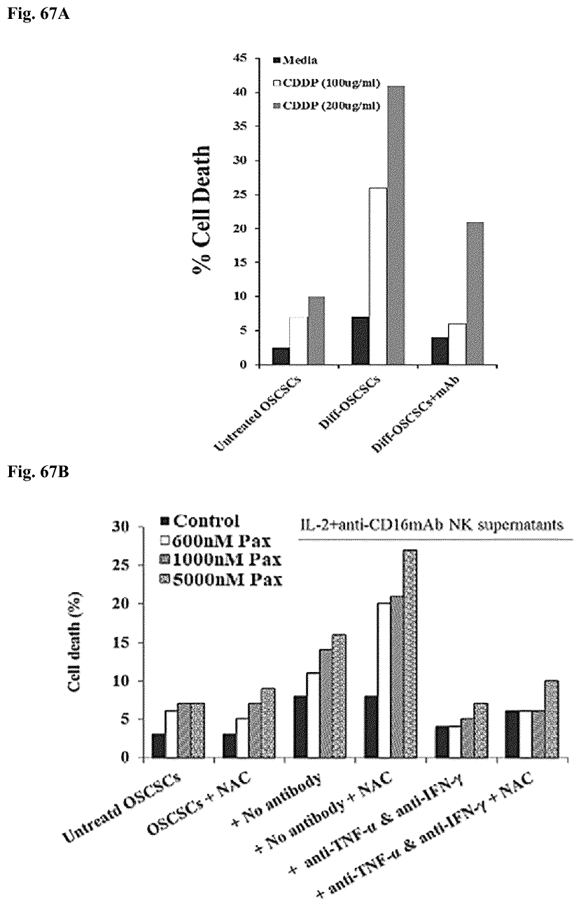

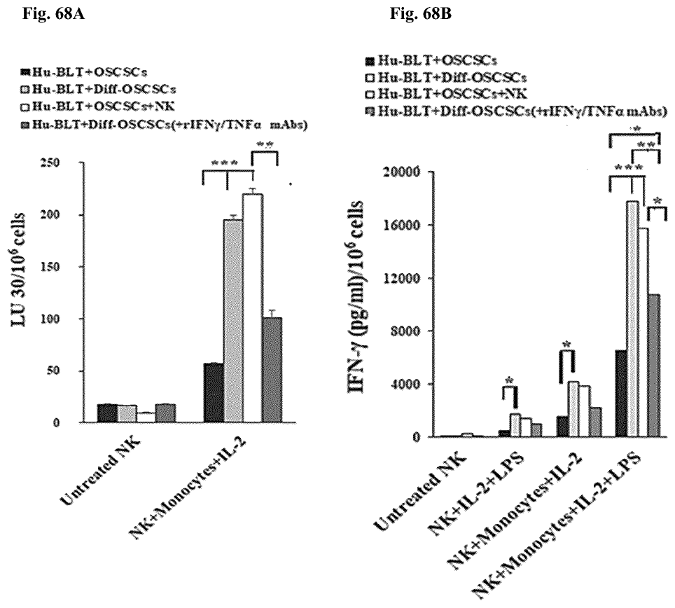

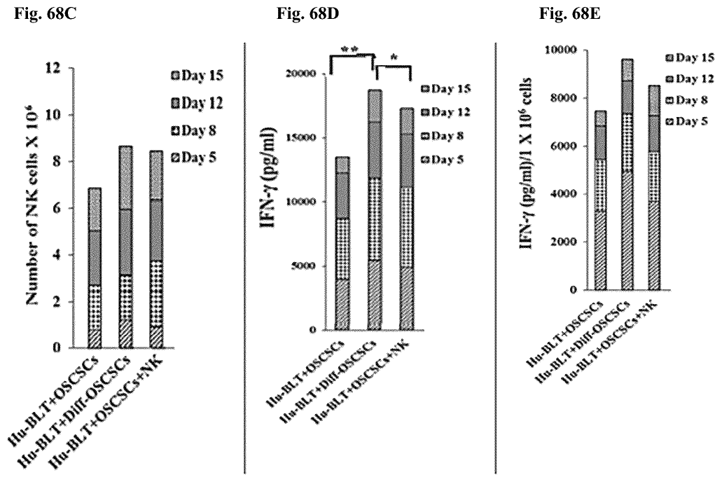

Hu-BLT mice exhibited greater than 96%-99% reconstitution with hCD45c immune cells in BM, spleen and 100 peripheral blood when considering contaminating mouse CD45c immune cells. The profile of CD3^{C}T cells (FIG. 16G), CD3^{C}CD4^{C}T cells (FIG. 16H) and CD3^{C}CD8^{C}T cells (FIG. 62I) in peripheral blood of hu-BLT mice resembled that of human peripheral blood; however, the percentage of B cells was slightly higher (FIG. 62J) while percentage of NK cells was less (FIG. 62K) in hu-BLT mice compared to humans.

Loss of NK Cytotoxicity and IFN-γ Secretion in Tumor-Bearing Mice within all Tissue Compartments, and Restoration with NK Injection and or Feeding AJ2 and Anti-PD1 Injection

Tumor-bearing mice exhibit lower NK-mediated cytotoxicity in splenocytes (FIG. 63A), BM-derived immune cells (FIG. 63B), PBMCs (FIG. 63C), and CD3* depleted splenocytes (FIG. 63D). NK-injected tumor-bearing mice, both alone and in combination with feeding AJ2 exhibited elevated NK cytotoxicity in all tissue compartments, with the highest increase observed when mice were fed AJ2, injected with NK cells and anti-PD1 antibody (FIG. 63A-FIG. 64D). When T cells were depleted from the splenocytes by CD3+ positive selection kit, similar profiles of NK cytotoxicity could be seen in CD3+ depleted splenocytes as observed in FIG. 63A (FIG. 63D). Splenocytes (FIG. 63E and Table 2), BM-derived immune cells (FIG. 63F), PBMCs (FIG. 63G), and CD3+ T cells from splenocytes (FIG. 63H) of 125 tumor-bearing hu-BLT mice secreted lower IFN-γ. Injection of anti-PD1 antibody in combination with NK cells and AJ2 either had no or decreased effect on IFN-γ secretion in all tissue compartments when compared to NK and AJ2 groups (FIG. 63E-FIG. 63G). NK-injected tumor-bearing mice showed elevated IFN-γ secretion within all tissue compartments, with the highest increase observed when mice were also fed AJ2 (FIG. 63E-FIG. 63H). When T cells were depleted from the splenocytes by CD3+ positive selection kit, the tumor-bearing mice that received NK injection had the highest increase in IFN-γ secretion as compared to tumor-bearing mice in the absence of NK injection, or NK injected non-tumor bearing control mice (FIG. 63I).

TABLE 2

| Spleen | IFN-γ | GRO | IL-12P70 | IL-17A | IL-1b | IL-6 | IL-8 | IP-10 | MCP-1 | MIP-1α | TNF-α |
|---|---|---|---|---|---|---|---|---|---|---|---|
| AJ2 control | 17 | 48 | 4 | 2 | 7 | 54 | 963 | 8 | 2134 | 289 | 51 |
| OSCSCs | 16 | 60 | 4 | 2 | 6 | 82 | 2108 | 9 | 2849 | 581 | 72 |
| OSCSCs + NK | 170 | 382 | 5 | 12 | 37 | 662 | 8702 | 21 | 7333 | 1009 | 88 |
| OSCSCs + NK + AJ2 | 322 | 353 | 6 | 22 | 30 | 564 | 15230 | 144 | 7764 | 1229 | 142 |

Increased Proportions of CD3⁺CD8⁺T Cells in Various Tissue Compartments when Hu-BLT Mice were Injected with NK Cells and Fed AJ2

Increased proportions of CD3⁺ or CD3⁺CD8⁺ T cells were seen in BM (FIG. 64A), spleen (FIG. 64B) and blood (data not shown) of mice injected with NK cells alone or in combination with AJ2 feeding (Table 3 and Table 4). When considering the percentages of T cells at the time of sacrifice, PBMCs, spleen and BM exhibited higher percentages of CD3⁺ T cells, and BM exhibited an elevation in HLADR⁺CD11B⁺ immune subsets in tumor-bearing mice injected with NK cells alone or in combination with AJ2 feeding (Table 3). Percentages of CD3⁺ T cells and CD3⁺CD8⁺T cells increased in tumor-bearing mice injected with NK cells alone or in combination with AJ2 feeding when BM and splenocytes were cultured (Table 3). Similarly, tumors dissociated from tumor-bearing mice injected with NK and/or fed AJ2 demonstrated elevated levels of CD3⁺T cells at the time of sacrifice (Table 4).

TABLE 3

| PBMCs | CD3 | Spleen | CD3 |
|---|---|---|---|
| AJ2 control | 66.0 | AJ2 control | 55.8 |
| OSCSCs | 44.6 | OSCSCs | 38.2 |
| OSCSCs + NK | 67.6 | OSCSCs + NK | 64.1 |
| OSCSCs +AJ2 | 50.2 | OSCSCs +AJ2 | 52.1 |
| OSCSCs + NK + AJ2 | 72.8 | OSCSCs + NK + AJ2 | 69.9 |

| BM | CD3 | HLADR + CD11b |
|---|---|---|
| AJ2 control | 2.14 | 10.2 |
| OSCSCs | 2.12 | 11.1 |
| OSCSCs + NK | 2.36 | 15.7 |
| OSCSCs + AJ2 | 1.79 | 11.2 |
| OSCSCs + NK + AJ2 | 5.98 | 16.1 |

TABLE 4

| Tumors Day 0 | |
|---|---|
| CD45+ | CD3 |
| OSCSCs | 56.3 |
| OSCSCs + NK | 81.0 |
| OSCSCs + AJ2 | 76.5 |
| OSCSCs + NK + AJ2 | 83.7 |

NK Injection and/or Feeding AJ2 Inhibit Growth and Progression of Stem-Like Oral Tumors, and Differentiate CSCs In Vivo in Hu-BLT Mice

Tumor cells from NSG (FIG. 65A) and hu-BLT mice with no NK injection grew rapidly, whereas those with NK cells did not grow or grew very slowly (FIG. 65A-FIG. 65C). Similarly, tumors from hu-BLT mice implanted with NK supernatant-differentiated tumors did not grow (FIG. 65C), and blocking tumor differentiation with the combination of anti-IFN-γ and anti-TNF-α antibodies before implantation restored tumor growth in vivo (FIG. 65B and FIG. 65C). Tumor growth was less in NK-injected mice fed AJ2 in comparison to NK alone-injected mice (FIG. 65C), and both were substantially less than those which only received oral tumor implantation (FIG. 65C). Nine to 11-fold more hCD45C immune cells infiltrated in tumors of tumor-bearing mice injected with NK cells when compared to tumor alone mice (FIG. 65D). The percentages of epithelial cells expressing surface EpCAM in tumors were approximately 5-fold higher from tumor-bearing mice without NK injection when compared to NK-injected tumor-bear. Significantly higher IFN-g secretion was obtained from tumors of tumor-bearing mice injected with NK cells at different days of cultures when compared to those from tumor-bearing hu-BLT or NSG mice (FIG. 65E). Expression of CD54 and MHC-I were higher on tumors obtained from NK-injected tumor-bearing mice as compared to hu-BLT and NSG mice, which only received tumors (FIG. 65F). Tumors from NK-injected tumor-bearing mice in the absence and presence of feeding with AJ2 were highly resistant to NK cell-mediated cytotoxicity when compared to tumors from tumor-bearing mice without NK injection demonstrating their differentiated phenotype (FIG. 65G and FIG. 65H). When NK-mediated differentiation of tumor cells was blocked with anti-IFN-γ and anti-TNF-α antibodies before implantation, the susceptibility of tumors to NK cell-mediated cytotoxicity was restored (FIG. 65H). Oral tumors from hu-BLT mice injected with NK cells secreted relatively less VEGF when standardized based on the secretion of VEGF from tumor-bearing mice (FIG. 65I). Tumors from NK-injected hu-BLT mice had higher levels of hCD45c infiltrating lymphocytes, and the highest increase were seen in those which were injected with NK cells and fed with AJ2 (FIG. 65J). The majority of infiltrating CD45c immune cells were CD3+ T cells with CD4+ T cell subsets having moderately higher proportions than CD8+ T cells, and demonstrating CD3+CD56+CD16c NKT subsets. When tumors were treated with IL-2, the high intensity CD4, CD8 and CD16/CD56 surface expression, which were down-regulated during interaction with tumors, was restored.

Sera from peripheral blood of NK-injected tumor-bearing hu-BLT mice exhibited increased IFN-γ secretion as well as other cytokines/chemokines/growth factors and ligands when compared to those from tumor-bearing hu-BLT mice without NK cells, and, in particular, IFN-γ secretion was further enhanced by feeding mice AJ2 (FIG. 66A and FIG. 66C).

CDDP or Paclitaxel with and without NAC Induce Significant Cell Death in OSCSCs Differentiated with IL-2+-Anti-CD16mAb Treated NK Supernatant

Differentiation of OSCSCs with NK supernatants resulted in significant susceptibility of tumors to CDDP (FIG. 67A). Similarly, paclitaxel mediated higher cell death of NK-supernatants differentiated OSCSCs, and NAC significantly increased Paclitaxel-mediated cell death (FIG. 67B). Blocking NK-mediated differentiation of OSCSCs with anti-IFN-γ and anti-TNF-α antibodies substantially decreased cell death induced by CDDP or paclitaxel with and without NAC (FIG. 67B). Treatment of OSCCs, patient-derived differentiated oral tumor, with CDDP or paclitaxel and NAC exhibited higher cell death.

Monocytes and Osteoclasts from NK Injected Tumor-Bearing Mice had Greater Ability to Activate NK Cells when Compared to Those from Tumor-Bearing Mice in the Absence of NK Injection

NK cells from NK injected tumor-bearing mice and NK supernatant-differentiated tumor-bearing mice when cultured with autologous monocytes had significantly augmented cytotoxicity (FIG. 68A) and IFN-γ secretion (FIG. 68B) when compared to those implanted with undifferentiated tumors in the absence of NK cell injection. Blocking NK-mediated differentiation of tumors through the addition of antibodies to TNF-α and IFN-γ before implantation decreased cytotoxicity (FIG. 68A), and IFN-γ secretion (FIG. 68B) of the NK cells. We then generated OCs from BM-derived monocytes of hu-BLT mice and cultured with allogeneic NK cells from healthy human donors to study the extent of NK cells expansion. NK cells expansion (FIG. 68C), and IFN-g secretion (FIG. 68D and FIG. 68E) were augmented in NK-injected tumor-bearing mice or NK supernatant-differentiated tumor-bearing mice when compared to those obtained from tumor-bearing mice without NK injection (FIG. 68C-FIG. 68E). Thus, monocytes and osteoclasts from NK injected tumor-bearing mice had greater ability to activate NK cells than those from tumor-bearing mice in the absence of NK injection.

### REFERENCES

1. Jewett A, Man Y G, Cacalano N, Kos J, Tseng H C: Natural killer cells as effectors of selection and differentiation of stem cells: role in resolution of inflammation. Journal of immunotoxicology 2014, 11(4):297-307.
2. Tseng H C, Bui V, Man Y G, Cacalano N, Jewett A: Induction of Split Anergy Conditions Natural Killer Cells to Promote Differentiation of Stem Cells through Cell-Cell Contact and Secreted Factors. Frontiers in immunology 2014, 5:269.
3. Burke S, Lakshmikanth T, Colucci F, Carbone E: New views on natural killer cell-based immunotherapy for melanoma treatment. Trends in immunology 2010, 31(9):339-345.
4. Larsen S K, Gao Y, Basse P H: NK cells in the tumor microenvironment. Critical reviews in oncogenesis 2014, 19(1-2):91-105.
5. Imai K, Matsuyama S, Miyake S, Suga K, Nakachi K: Natural cytotoxic activity of peripheral-blood lymphocytes and cancer incidence: an 11-year follow-up study of a general population. Lancet 2000, 356(9244):1795-1799.
6. Bruno A, Ferlazzo G, Albini A, Noonan D M: A think tank of TINK/TANKs: tumor-infiltrating/tumor-associated natural killer cells in tumor progression and angiogenesis. Journal of the National Cancer Institute 2014, 106(8):dju200.
7. Gross E, Sunwoo J B, Bui J D: Cancer immunosurveillance and immunoediting by natural killer cells. Cancer journal (Sudbury, Mass) 2013, 19(6):483-489.
8. Mirjacic Martinovic K M, Babovic N, Dzodic R R, Jurisic V B, Tanic N T, Konjevic G M: Decreased expression of NKG2D, NKp46, DNAM-1 receptors, and intracellular perforin and STAT-1 effector molecules in NK cells and their dim and bright subsets in metastatic melanoma patients. Melanoma research 2014, 24(4):295-304.
9. Gubbels J A, Felder M, Horibata S, Belisle J A, Kapur A, Holden H, Petrie S, Migneault M, Rancourt C, Connor J P et al: MUC16 provides immune protection by inhibiting synapse formation between NK and ovarian tumor cells. Molecular cancer 2010, 9:11.
10. Balsamo M, Scordamaglia F, Pietra G, Manzini C, Cantoni C, Boitano M, Queirolo P, Vermi W, Facchetti F, Moretta A et al: Melanoma-associated fibroblasts modulate NK cell phenotype and antitumor cytotoxicity. Proceedings of the National Academy of Sciences of the U.S. Pat. No. 2,009,106(49):20847-20852.
11. Castriconi R, Cantoni C, Della Chiesa M, Vitale M, Marcenaro E, Conte R, Biassoni R, Bottino C, Moretta L, Moretta A: Transforming growth factor beta 1 inhibits expression of NKp30 and NKG2D receptors: consequences for the NK-mediated killing of dendritic cells. Proceedings of the National Academy of Sciences of the U.S. Pat. No. 2,003,100(7):4120-4125.
12. Pietra G, Manzini C, Rivara S, Vitale M, Cantoni C, Petretto A, Balsamo M, Conte R, Benelli R, Minghelli S et al: Melanoma cells inhibit natural killer cell function by modulating the expression of activating receptors and cytolytic activity. Cancer research 2012, 72(6):1407-1415.
13. Krockenberger M, Dombrowski Y, Weidler C, Ossadnik M, Honig A, Hausler S, Voigt H, Becker J C, Leng L, Steinle A et al: Macrophage migration inhibitory factor contributes to the immune escape of ovarian cancer by down-regulating NKG2D. Journal of immunology (Baltimore, Md: 1950) 2008, 180(11):7338-7348.
14. Vitale M, Cantoni C, Pietra G, Mingari M C, Moretta L: Effect of tumor cells and tumor microenvironment on NK-cell function. European journal of immunology 2014, 44(6):1582-1592.
15. Gallois A, Silva I, Osman I, Bhardwaj N: Reversal of natural killer cell exhaustion by TIM-3 blockade. Oncoimmunology 2014, 3(12):e946365.
16. Hersey P, Edwards A, Honeyman M, McCarthy W H: Low natural-killer-cell activity in familial melanoma patients and their relatives. British journal of cancer 1979, 40(1):113-122.
17. Perussia B, Ramoni C, Anegon I, Cuturi M C, Faust J, Trinchieri G: Preferential proliferation of natural killer cells among peripheral blood mononuclear cells cocultured with B lymphoblastoid cell lines. Natural immunity and cell growth regulation 1987, 6(4):171-188.
18. Rabinowich H, Sedlmayr P, Herberman R B, Whiteside T L: Increased proliferation, lytic activity, and purity of human natural killer cells cocultured with mitogen-activated feeder cells.

Cellular immunology 1991, 135(2):454-470.
19. Igarashi T, Wynberg J, Srinivasan R, Becknell B, McCoy J P, Jr., Takahashi Y, Suffredini D A, Linehan W M, Caligiuri M A, Childs R W: Enhanced cytotoxicity of allogeneic NK cells with killer immunoglobulin-like receptor ligand incompatibility against melanoma and renal cell carcinoma cells. Blood 2004, 104(1):170-177.
20. Srivastava S, Lundqvist A, Childs R W: Natural killer cell immunotherapy for cancer: a new hope. Cytotherapy 2008, 10(8):775-783.
21. Gras Navarro A, Bjorklund A, Chekenya M: Therapeutic potential and challenges of Natural killer cells in treatment of solid tumors. Frontiers in Immunology 2015, 6.
22. Alici E, Sutlu T, Bjorkstrand B, Gilljam M, Stellan B, Nahi H, Quezada H C, Gahrton G, Ljunggren H G, Dilber M S: Autologous antitumor activity by NK cells expanded from myeloma patients using GMP-compliant components. Blood 2008, 111(6):3155-3162.
23. Fujisaki H, Kakuda H, Shimasaki N, Imai C, Ma J, Lockey T, Eldridge P, Leung W H, Campana D: Expansion of highly cytotoxic human natural killer cells for cancer cell therapy.

Cancer research 2009, 69(9):4010-4017.
24. Berg M, Lundqvist A, McCoy P, Jr., Samsel L, Fan Y, Tawab A, Childs R: Clinical-grade ex vivo-expanded human natural killer cells up-regulate activating receptors and death receptor ligands and have enhanced cytolytic activity against tumor cells. Cytotherapy 2009, 11(3):341-355.
25. Garg T K, Szmania S M, Khan J A, Hoering A, Malbrough P A, Moreno-Bost A, Greenway A D, Lingo J D, Li X, Yaccoby S et al: Highly activated and expanded natural killer cells for multiple myeloma immunotherapy. Haematologica 2012, 97(9):1348-1356.
26. Imai C, Iwamoto S, Campana D: Genetic modification of primary natural killer cells overcomes inhibitory signals and induces specific killing of leukemic cells. Blood 2005, 106(1):376-383.
27. Lapteva N, Durett A G, Sun J, Rollins L A, Huye L L, Fang J, Dandekar V, Mei Z, Jackson K, Vera J et al: Large-scale ex vivo expansion and characterization of natural killer cells for clinical applications. Cytotherapy 2012, 14(9):1131-1143.
28. Koepsell S A, Miller J S, McKenna D H, Jr.: Natural killer cells: a review of manufacturing and clinical utility. Transfusion 2013, 53(2):404-410.
29. Miller J S, Soignier Y, Panoskaltsis-Mortari A, McNearney S A, Yun G H, Fautsch S K, McKenna D, Le C, Defor T E, Burns L J et al: Successful adoptive transfer and in vivo expansion of human haploidentical NK cells in patients with cancer. Blood 2005, 105(8):3051-3057.
30. Iliopoulou E G, Kountourakis P, Karamouzis M V, Doufexis D, Ardavanis A, Baxevanis C N, Rigatos G, Papamichail M, Perez S A: A phase I trial of adoptive transfer of allogeneic natural killer cells in patients with advanced non-small cell lung cancer. Cancer immunology, immunotherapy: CII 2010, 59(12):1781-1789.
31. Sotiropoulou P A, Perez S A, Gritzapis A D, Baxevanis C N, Papamichail M: Interactions between human mesenchymal stem cells and natural killer cells. Stem cells 2006, 24(1):74-85.
32. Geller M A, Cooley S, Judson P L, Ghebre R, Carson L F, Argenta P A, Jonson A L, Panoskaltsis-Mortari A, Curtsinger J, McKenna D et al: A phase II study of allogeneic natural killer cell therapy to treat patients with recurrent ovarian and breast cancer. Cytotherapy 2011, 13(1):98-107.
33. Tseng H C, Arasteh A, Paranjpe A, Teruel A, Yang W, Behel A, Alva J A, Walter G, Head C, Ishikawa T O et al: Increased lysis of stem cells but not their differentiated cells by natural killer cells; de-differentiation or reprogramming activates NK cells. PloS one 2010, 5(7):e11590.
34. Tseng H C, Inagaki A, Bui V T, Cacalano N, Kasahara N, Man Y G, Jewett A: Differential Targeting of Stem Cells and Differentiated Glioblastomas by NK Cells. Journal of Cancer 2015, 6(9):866-876.
35. Bui V T, Tseng H-C, Maung P O, Kozlowska A, Mann K, Topchyan P, Jewett A: Augmented IFN-γ and TNF-α Induced by Probiotic Bacteria in NK Cells Mediate Differentiation of Stem-Like Tumors Leading to Inhibition of Tumor Growth and Reduction in Inflammatory Cytokine Release; Regulation by IL-10. Frontiers in immunology 2015, 6.
36. Bui V T, Tseng H C, Kozlowska A, Maung P O, Kaur K, Topchyan P, Jewett A: Augmented IFN-gamma and TNF-alpha Induced by Probiotic Bacteria in NK Cells Mediate Differentiation of Stem-Like Tumors Leading to Inhibition of Tumor Growth and Reduction in Inflammatory Cytokine Release; Regulation by IL-10. Front Immunol 2015, 6:576.
37. Jewett A, Bonavida B: Target-induced inactivation and cell death by apoptosis in a subset of human NK cells. Journal of immunology (Baltimore, Md: 1950) 1996, 156(3):907-915.
38. Shimizu S, Hong P, Arumugam B, Pokomo L, Boyer J, Koizumi N, Kittipongdaja P, Chen A, Bristol G, Galic Z et al: A highly efficient short hairpin RNA potently down-regulates CCR5 expression in systemic lymphoid organs in the hu-BLT mouse model. Blood 2010, 115(8):1534-1544.
39. Vatakis D N, Koya R C, Nixon C C, Wei L, Kim S G, Avancena P, Bristol G, Baltimore D, Kohn D B, Ribas A et al: Antitumor activity from antigen-specific CD8 T cells generated in vivo from genetically engineered human hematopoietic stem cells. Proceedings of the National Academy of Sciences of the U.S. Pat. No. 2,011,108(51):E1408-1416.
40. Kozlowska A K, Kaur K, Topchyan P, Jewett A: Adoptive transfer of osteoclast-expanded natural killer cells for immunotherapy targeting cancer stem-like cells in humanized mice. Cancer immunology, immunotherapy: CII 2016.
41. Jewett A, Cavalcanti M, Bonavida B: Pivotal role of endogenous TNF-alpha in the induction of functional inactivation and apoptosis in NK cells. Journal of immunology (Baltimore, Md: 1950) 1997, 159(10):4815-4822.
42. Jewett A, Bonavida B: Interferon-alpha activates cytotoxic function but inhibits interleukin-2-mediated proliferation and tumor necrosis factor-alpha secretion by immature human natural killer cells. J Clin Immunol 1995, 15(1):35-44.
43. Jewett A, Wang M Y, Teruel A, Poupak Z, Bostanian Z, Park N H: Cytokine dependent inverse regulation of CD54 (ICAM1) and major histocompatibility complex class I antigens by nuclear factor kappaB in HEp2 tumor cell line: effect on the function of natural killer cells. Human immunology 2003, 64(5):505-520.
44. Tseng H C, Kanayama K, Kaur K, Park S H, Park S, Kozlowska A, Sun S, McKenna C E, Nishimura I, Jewett A: Bisphosphonate-induced differential modulation of immune cell function in gingiva and bone marrow in vivo: Role in osteoclast-mediated NK cell activation. Oncotarget 2015, 6(24):20002-20025.
45. Tam Y K, Martinson J A, Doligosa K, Klingemann H G: Ex vivo expansion of the highly cytotoxic human natural killer-92 cell-line under current good manufacturing practice conditions for clinical adoptive cellular immunotherapy. Cytotherapy 2003, 5(3):259-272.
46. Magister S, Tseng H C, Bui V T, Kos J, Jewett A: Regulation of split anergy in natural killer cells by inhibition of cathepsins C and H and cystatin F. Oncotarget 2015, 6(26):22310-22327.
47. Perisic Nanut M, Sabotic J, Jewett A, Kos J: Cysteine cathepsins as regulators of the cytotoxicity of NK and T cells. Front Immunol 2014, 5:616.
48. Cantoni C, Huergo-Zapico L, Parodi M, Pedrazzi M, Mingari M C, Moretta A: NK Cells, Tumor Cell Transition, and Tumor Progression in Solid Malignancies: New Hints for NK-Based Immunotherapy? 2016, 2016:4684268.
49. Tallerico R, Garofalo C, Carbone E: A New Biological Feature of Natural Killer Cells: The Recognition of Solid Tumor-Derived Cancer Stem Cells. Front Immunol 2016, 7:179.
50. Kamiya T, Chang Y H, Campana D: Expanded and Activated Natural Killer Cells for Immunotherapy of Hepatocellular Carcinoma. Cancer immunology research 2016.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-82. (canceled)

83. A method of activating a NK cell, comprising:
treating the NK cell with IL-2 and anti-CD16 antibody; and
culturing the treated NK cell in a medium comprising osteoclast cell (OC) cells, thereby activating the NK cell.

84. The method of claim 83, further comprising the step of isolating the NK cell from peripheral blood-derived mononuclear cells (PBMCs).

85. The method of claim 84, wherein the peripheral blood-derived mononuclear cells (PBMCs) are from a healthy donor or cancer patient.

86. The method of claim 83, wherein the medium further comprises one or more probiotic bacteria.

87. The method of claim 86, wherein the one or more probiotic includes at least one bacterial strain selected from the group consisting of Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus.

88. The method of claim 86, wherein the ratio of the probiotic bacteria concentration to the NK cell and/or the OC concentrations in the cell culture is
i) at least 1:2 for NK cells:probiotic bacteria;
ii) at least 1:4 for OCs:probiotic bacteria; and/or
iii) at least 1:2:4 for OCs:NK cells:probiotic bacteria.

89. The method of claim 83, wherein the osteoclast cell is allogenic or autologous.

90. The method of claim 83, wherein the osteoclast cell is live or irradiated.

91. A method of treating cancer or a cancer-related disease or disorder in a subject having or suspected of having a cancer or cancer-related disease or disorder, comprising administering to the subject a therapeutically effective amount of NK cells activated according to the method of claim 83.

92. The method of claim 91, further comprising the step of administering to the subject a therapeutically effective amount of one or more probiotic bacteria.

93. The method of claim 92, wherein the one or more probiotic includes at least one bacterial strain selected from the group consisting of Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus.

94. The method of claim 92, wherein the one or more probiotic bacteria are administered at about 48 hours after administration of the therapeutically effective amount of NK cells.

95. The method of claim 91, further comprising the step of administering to the subject a therapeutically effective amount of an anti-PD1 infusion.

96. The method of claim 91, wherein the activated NK cells are cryopreserved or freshly generated prior to administration.

97. The method of claim 91, wherein the clinical benefit rate is at least 25% at six months after administration.

98. The method of claim 91, further comprising the step of administering to the subject a chemotherapeutic agent.

99. A composition for the treatment of cancer in a subject in need thereof, the composition comprising:
an activated NK cell of claim 83; and
optionally, at least one chemotherapeutic agent.
